(19) United States
(12) Patent Application Publication
(10) Pub. No.: US 2016/0072072 A1
(43) Pub. Date: Mar. 10, 2016

(54) CONDENSED CYCLIC COMPOUND AND ORGANIC LIGHT-EMITTING DEVICE INCLUDING THE SAME

(71) Applicant: SAMSUNG DISPLAY CO., LTD., Yongin-City (KR)

(72) Inventors: Sooyon KIM, Yongin-City (KR); Haejin KIM, Yongin-City (KR); Mieun JUN, Yongin-City (KR); Kwanghyun KIM, Yongin-City (KR); Youngkook KIM, Yongin-City (KR); Seokhwan HWANG, Yongin-City (KR)

(21) Appl. No.: 14/669,137

(22) Filed: Mar. 26, 2015

(30) Foreign Application Priority Data
Sep. 5, 2014 (KR) 10-2014-0119378

(51) Int. Cl. H01L 51/00; C07F 7/08; C09K 11/06; C07D 405/04; C07D 333/50; C07D 409/04; C07D 307/77; C07D 407/04

(52) U.S. Cl. CPC H01L 51/0052; C07D 307/77; C07F 7/0812; C07D 407/04; C07D 405/04; C07D 333/50; C07D 409/04; H01L 51/0073; H01L 51/0058; H01L 51/0094; H01L 51/0067; H01L 51/0074; C09K 11/06; C09K 2211/1088; C09K 2211/1007; C09K 2211/1011; C09K 2211/1092; C09K 2211/1029; H01L 51/5012

(57) ABSTRACT

A condensed cyclic compound is represented by Formula 1:

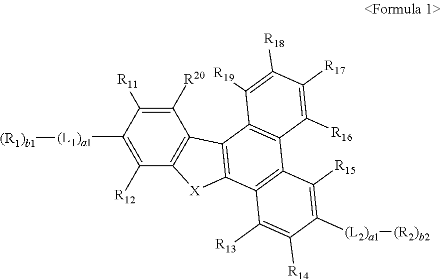

wherein X, R₁ to R₂₀, L₁, L₂, a1, a2, b1, and b2 are as defined in the specification. An organic light-emitting device includes the condensed cyclic compound.

### CROSS-REFERENCE TO RELATED APPLICATION

Korean Patent Application No. 10-2014-0119378, filed on Sep. 5, 2014, in the Korean Intellectual Property Office, and entitled: “Condensed Cyclic Compound and Organic Light-Emitting Device Including the Same,” is incorporated by reference herein in its entirety.

### BACKGROUND

1. Field

Embodiments relate to a condensed-cyclic compound and an organic light-emitting device including the same.

2. Description of the Related Art

Organic light-emitting devices are self-emitting devices that have wide viewing angles, high contrast ratios, quick response times, high brightness, and excellent driving voltage characteristics, and can produce multicolored images.

The organic light-emitting device may include a first electrode disposed on a substrate, and a hole transport region, an emission layer, an electron transport region, and a second electrode, which are sequentially disposed on the first electrode. Holes provided from the first electrode are transported to the emission layer through the hole transport region, and electrons provided from the second electrode are transported to the emission layer through the electron transport region. Carriers, such as the holes and electrons, may recombine in the emission layer to generate excitons. When the excitons drop from an excited state to a ground state, light is emitted.

### SUMMARY

Embodiments are directed to a condensed cyclic compound is represented by Formula 1:

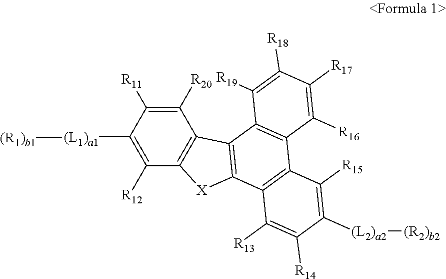

wherein, in Formula 1,

X is O or S;

L₁ and L₂ are each independently selected from a substituted or unsubstituted C₃-C₁₀ cycloalkylene group, a substituted or unsubstituted C₁-C₁₀ heterocycloalkylene group, a substituted or unsubstituted C₃-C₁₀ cycloalkenylene group, a substituted or unsubstituted C₁-C₁₀ heterocycloalkenylene group, a substituted or unsubstituted C₆-C₆₀ arylene group, a substituted or unsubstituted C₁-C₆₀ heteroarylene group, a substituted or unsubstituted divalent non-aromatic condensed polycyclic group, and a substituted or unsubstituted divalent non-aromatic condensed heteropolycyclic group;

a1 and a2 are each independently selected from 0, 1, 2, and 3, and when a1 is 2 or greater, a plurality of L₁ are identical to or different from each other, and when a2 is 2 or greater, a plurality of L₂ are identical to or different from each other;

R₁ and R₂ are each independently selected from a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —Si(Q₁)(Q₂)(Q₃), —B(Q₄)(Q₅), —N(Q₆)(Q₇), and —C(Q₈)═N(Q₉);

b1 and b2 are each independently an integer selected from 1 to 4, and when b1 is 2 or greater, a plurality of R₁ are identical to or different from each other, and when b2 is 2 or greater, a plurality of R₂ are identical to or different from each other;

R₁₁ to R₂₀ are each independently selected from a hydrogen, a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —Si(Q₁)(Q₂)(Q₃), —B(Q₄)(Q₅), and —N(Q₆)(Q₇);

at least one substituent of the substituted C₃-C₁₀ cycloalkylene group, substituted C₁-C₁₀ heterocycloalkylene group, substituted C₃-C₁₀ cycloalkenylene group, substituted C₁-C₁₀ heterocycloalkenylene group, substituted C₆-C₆₀ arylene group, substituted C₁-C₆₀ heteroarylene group, substituted a divalent non-aromatic condensed polycyclic group, substituted a divalent non-aromatic condensed heteropolycyclic group, substituted C₁-C₆₀ alkyl group, substituted C₂-C₆₀ alkenyl group, substituted C₂-C₆₀ alkynyl group, substituted C₁-C₆₀ alkoxy group, substituted C₃-C₁₀ cycloalkyl group, substituted C₁-C₁₀ heterocycloalkyl group, substituted C₃-C₁₀ cycloalkenyl group, substituted C₁-C₁₀ heterocycloalkenyl group, substituted C₆-C₆₀ aryl group, substituted C₆-C₆₀ aryloxy group, substituted C₆-C₆₀ arylthio group, substituted C₁-C₆₀ heteroaryl group, substituted monovalent non-aromatic condensed polycyclic group, and substituted monovalent non-aromatic condensed heteropolycyclic group is selected from,

a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, and a C₁-C₆₀ alkoxy group;

a C₁-C₆₀ alkyl group, a C₂-C₆₀ alkenyl group, a C₂-C₆₀ alkynyl group, and a C₁-C₆₀ alkoxy group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —Si(Q₁₁)(Q₁₂)(Q₁₃), —B(Q₁₄)(Q₁₅), and —N(Q₁₆)(Q₁₇);

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group;

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —Si(Q₂₁)(Q₂₂)(Q₂₃), —B(Q₂₄)(Q₂₅), and —N(Q₂₆)(Q₂₇); and

—Si(Q₃₁)(Q₃₂)(Q₃₃), —B(Q₃₄)(Q₃₅), and —N(Q₃₆)(Q₃₇),

wherein Q₁ to Q₉, Q₁₁ to Q₁₇, Q₂₁ to Q₂₇, and Q₃₁ to Q₃₇ are each independently selected from a hydrogen, a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group.

Embodiments are also directed to an organic light-emitting device that includes a first electrode, a second electrode facing the first electrode, and an organic layer between the first electrode and the second electrode. The organic layer includes an emission layer. The organic layer includes the condensed cyclic compound.

### BRIEF DESCRIPTION OF THE DRAWINGS

Features will become apparent to those of skill in the art by describing in detail exemplary embodiments with reference to the attached drawings in which:

FIG. 1 illustrates a schematic view of an organic light-emitting device according to an exemplary embodiment.

### DETAILED DESCRIPTION

Example embodiments will now be described more fully hereinafter with reference to the accompanying drawings; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing figures, the dimensions of layers and regions may be exaggerated for clarity of illustration. Like reference numerals refer to like elements throughout.

As used herein, the term “and/or” includes any and all combinations of one or more of the associated listed items. Expressions such as “at least one of,” when preceding a list of elements, modify the entire list of elements and do not modify the individual elements of the list.

A condensed cyclic compound is represented by Formula 1:

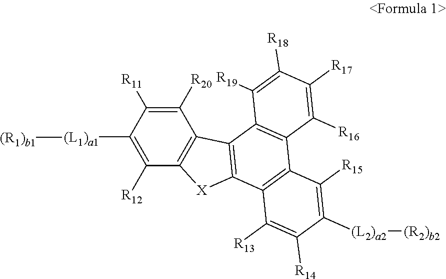

In Formula 1, X may be O or S.

In Formula 1, L₁ and L₂ may be each independently selected from a substituted or unsubstituted C₃-C₁₀ cycloalkylene group, a substituted or unsubstituted C₁-C₁₀ heterocycloalkylene group, a substituted or unsubstituted C₃-C₁₀ cycloalkenylene group, a substituted or unsubstituted C₁-C₁₀ heterocycloalkenylene group, a substituted or unsubstituted C₆-C₆₀ arylene group, a substituted or unsubstituted C₁-C₆₀ heteroarylene group, a substituted or unsubstituted divalent non-aromatic condensed polycyclic group, and a substituted or unsubstituted divalent non-aromatic condensed heteropolycyclic group.

For example, in Formula 1, L₁ and L₂ may be each independently selected from:

a phenylene group, a pentalenylene group, an indenylene group, a naphthylene group, an azulenylene group, a heptalenylene group, an indacenylene group, an acenaphthylene group, a fluorenylene group, a spiro-fluorenylene group, a benzofluorenylene group, a dibenzofluorenylene group, a phenalenylene group, a phenanthrenylene group, an anthracenylene group, a fluoranthenylene group, a triphenylenylene group, a pyrenylene group, a chrysenylene group, a naphthacenylene group, a picenylene group, a perylenylene group, a pentaphenylene group, a hexacenylene group, a pentacenylene group, a rubicenylene group, a coronenylene group, an ovalenylene group, a pyrrolylene group, a thiophenylene group, a furanylene group, an imidazolylene group, a pyrazolylene group, a thiazolylene group, an isothiazolylene group, an oxazolylene group, a isoxazolylene group, a pyridinylene group, a pyrazinylene group, a pyrimidinylene group, a pyridazinylene group, an isoindolylene group, an indolylene group, an indazolylene group, a purinylene group, a quinolinylene group, an isoquinolinylene group, a benzoquinolinylene group, a phthalazinylene group, a naphthyridinylene group, a quinoxalinylene group, a quinazolinylene group, a cinnolinylene group, a carbazolylene group, a phenanthridinylene group, an acridinylene group, a phenanthrolinylene group, a phenazinylene group, a benzoimidazolylene group, a benzofuranylene group, a benzothiophenylene group, an isobenzothiazolylene group, a benzooxazolylene group, an isobenzooxazolylene group, a triazolylene group, a tetrazolylene group, an oxadiazolylene group, a triazinylene group, a dibenzofuranylene group, a dibenzothiophenylene group, a benzocarbazolylene group, a dibenzocarbazolylene group, a thiadiazolylene group, an imidazopyridinylene group, and an imidazopyrimidinylene group; and

a phenylene group, a pentalenylene group, an indenylene group, a naphthylene group, an azulenylene group, a heptalenylene group, an indacenylene group, an acenaphthylene group, a fluorenylene group, a spiro-fluorenylene group, a benzofluorenylene group, a dibenzofluorenylene group, a phenalenylene group, a phenanthrenylene group, an anthracenylene group, a fluoranthenylene group, a triphenylenylene group, a pyrenylene group, a chrysenylene group, a naphthacenylene group, a picenylene group, a perylenylene group, a pentaphenylene group, a hexacenylene group, a pentacenylene group, a rubicenylene group, a coronenylene group, an ovalenylene group, a pyrrolylene group, a thiophenylene group, a furanylene group, an imidazolylene group, a pyrazolylene group, a thiazolylene group, an isothiazolylene group, an oxazolylene group, a isoxazolylene group, a pyridinylene group, a pyrazinylene group, a pyrimidinylene group, a pyridazinylene group, an isoindolylene group, an indolylene group, an indazolylene group, a purinylene group, a quinolinylene group, an isoquinolinylene group, a benzoquinolinylene group, a phthalazinylene group, a naphthyridinylene group, a quinoxalinylene group, a quinazolinylene group, a cinnolinylene group, a carbazolylene group, a phenanthridinylene group, an acridinylene group, a phenanthrolinylene group, a phenazinylene group, a benzoimidazolylene group, a benzofuranylene group, a benzothiophenylene group, an isobenzothiazolylene group, a benzooxazolylene group, an isobenzooxazolylene group, a triazolylene group, a tetrazolylene group, an oxadiazolylene group, a triazinylene group, a dibenzofuranylene group, a dibenzothiophenylene group, a benzocarbazolylene group, a dibenzocarbazolylene group, a thiadiazolylene group, an imidazopyridinylene group, and an imidazopyrimidinylene group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzoxazolyl group, an isobenzooxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, a thiadiazolyl group, an imidazopyridinyl group, and an imidazopyrimidinyl group.

According to some implementation, in Formula 1, L₁ and L₂ may be each independently a group represented by one of Formulae 3-1 to 3-32 below:

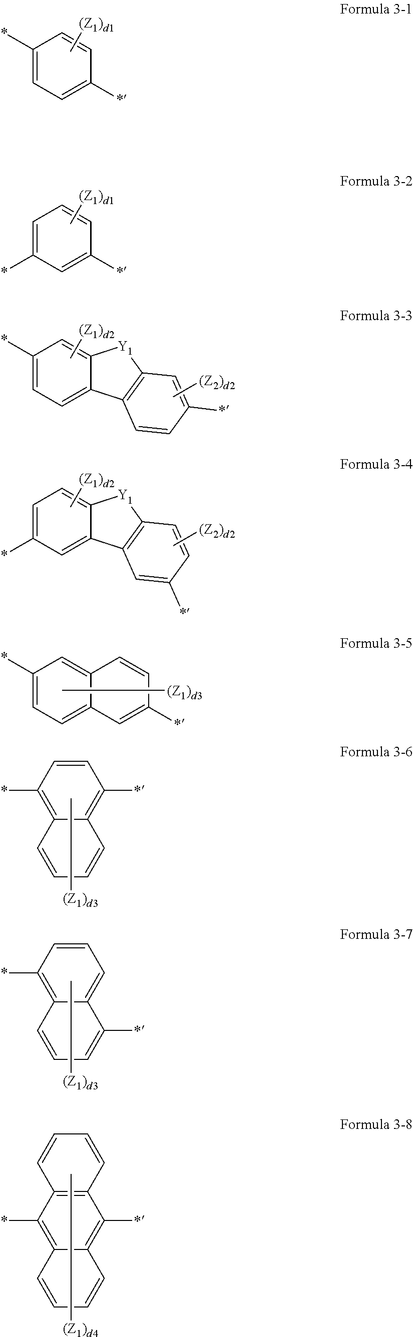

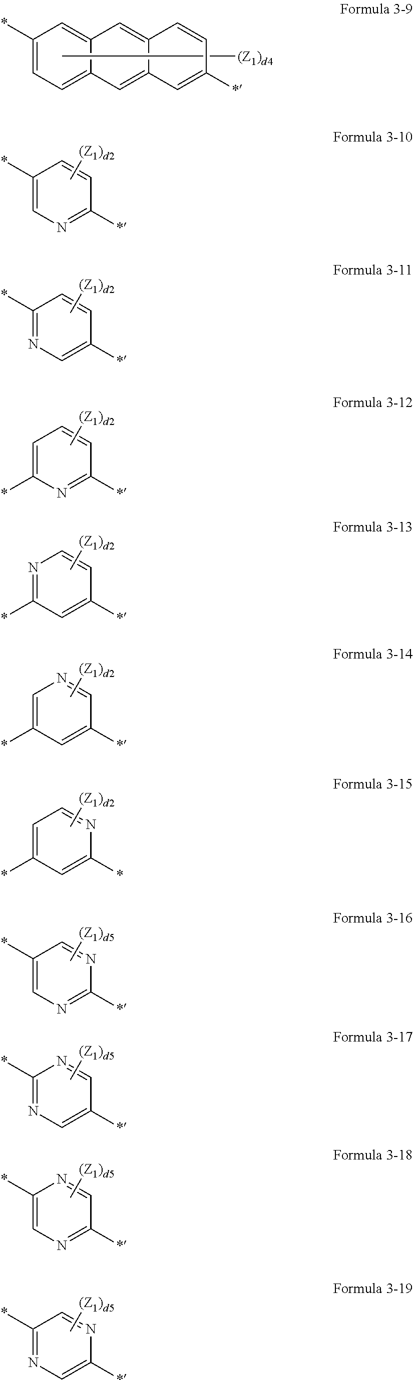

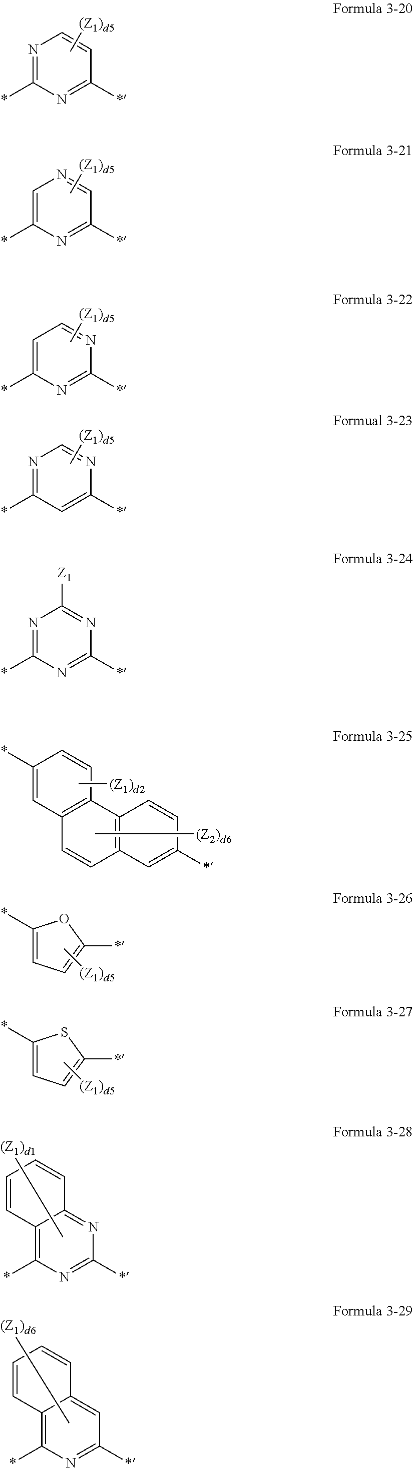

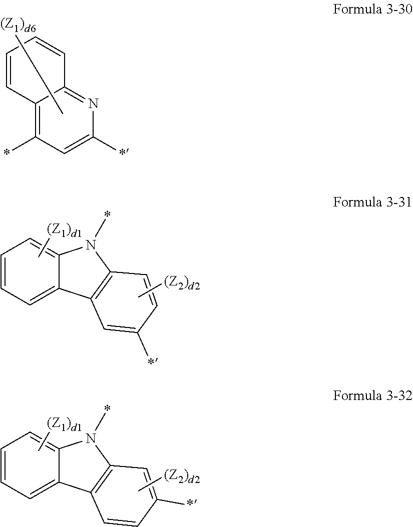

In Formulae 3-1 to 3-32,

Y₁ may be O, S, C(Z₃)(Z₄), N(Z₅), or Si(Z₆)(Z₇);

Z₁ to Z₇ may be each independently selected from a hydrogen, a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₁-C₂₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group,

d1 may be an integer selected from 1 to 4, d2 may be an integer selected from 1 to 3, d3 may be an integer selected from 1 to 6, d4 may be an integer selected from 1 to 8, d5 may be 1 or 2, d6 may be an integer selected from 1 to 5, and * and *′ are binding sites to a neighboring atom.

According to some implementations, in Formula 1, L₁ and L₂ may be each independently a group represented by Formulae 4-1 to 4-23 below:

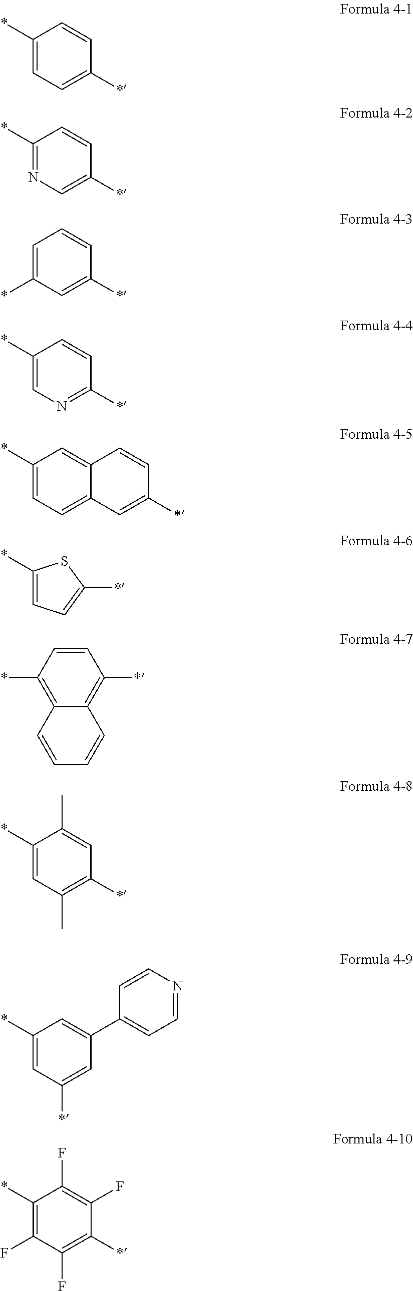

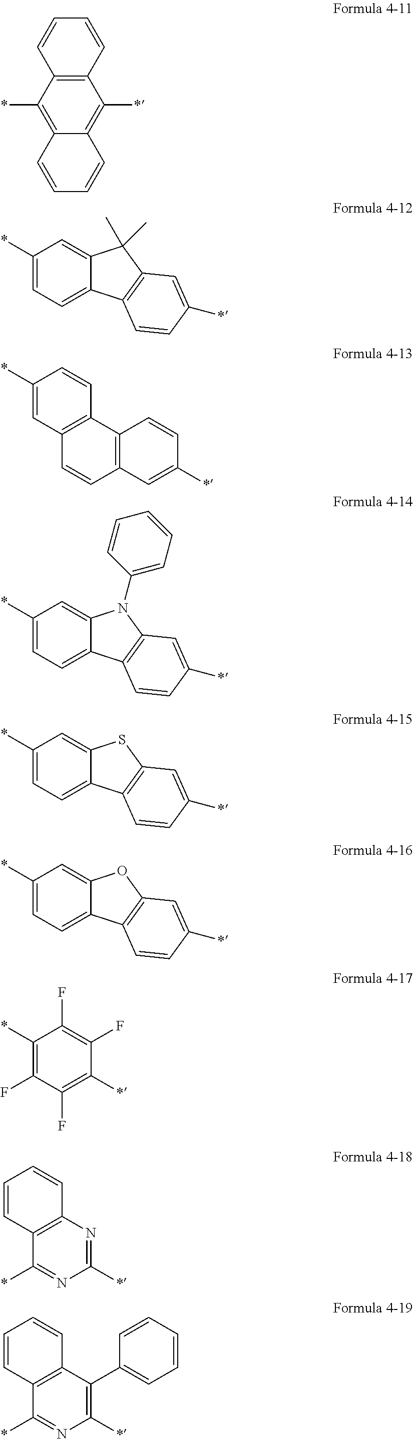

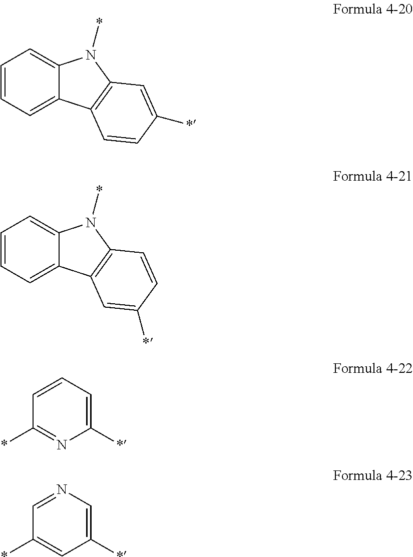

In Formulae 4-1 to 4-23, * and *′ are binding sites to a neighboring atom.

In Formula 1, a1 denotes the number of L₁ which may be selected from 0, 1, 2, and 3. For example, a1 may be 0 or 1. When a1 is 0, *-(L₁)ₐ₁-*′ is a single bond. When a1 is 2 or greater, a plurality of L₁ may be identical to or different from each other. In Formula 1, a2 is defined the same in connection with a1 and a structure of Formula 1.

For example, in Formula 1, a1 and a2 may be each independently 0 or 1.

In Formula 1, R₁ and R₂ may be each independently selected from:

a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted heterocycloalkenyl group, a substituted or unsubstituted C₆-C₆₀ aryl group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —Si(Q₁)(Q₂)(Q₃), —B(Q₄)(Q₅), —N(Q₆)(Q₇), and —C(Q₈)=N(Q₉).

For example, in Formula 1, R₁ and R₂ may be each independently selected from:

a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzoxazolyl group, an isobenzooxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, a thiadiazolyl group, an imidazopyridinyl group, and an imidazopyrimidinyl group;

a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzoxazolyl group, an isobenzooxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, a thiadiazolyl group, an imidazopyridinyl group and an imidazopyrimidinyl group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzoxazolyl group, an isobenzooxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, a thiadiazolyl group, an imidazopyridinyl group, an imidazopyrimidinyl group, and —Si(Q₃₁)(Q₃₂)(Q₃₃); and

—Si(Q₁)(Q₂)(Q₃) and —C(Q₈)=N(Q₉),

wherein Q₁ to Q₃, Q₈, Q₉, and Q₃₁ to Q₃₃ may be each independently selected from a hydrogen, a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, C₁-C₂₀ alkyl group, C₁-C₂₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group.

For example, in Formula 1, R₁ and R₂ may be each independently selected from:

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, a triazinyl group, a dibenzofuranyl group, a dibenzothiophenyl group, a benzocarbazolyl group, and a dibenzocarbazolyl group;

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, a triazinyl group, a dibenzofuranyl group, a dibenzothiophenyl group, a benzocarbazolyl group, and a dibenzocarbazolyl group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₁-C₂₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, a triazinyl group, a dibenzofuranyl group, a dibenzothiophenyl group, a benzocarbazolyl group, a dibenzocarbazolyl group, and —Si(Q₃₁)(Q₃₂)(Q₃₃); and

—Si(Q₁)(Q₂)(Q₃) and —C(Q₈)=N(Q₉);

wherein Q₁ to Q₃, Q₈, Q₉ and Q₃₁ to Q₃₃ may be each independently selected from a hydrogen, a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₁-C₂₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group, as examples.

According to some embodiments, in Formula 1, R₁ and R₂ may be each independently a group represented by one of *—Si(Q₁)(Q₂)(Q₃), *—C(Q₈)=N(Q₉), and Formulae 5-1 to 5-14 below:

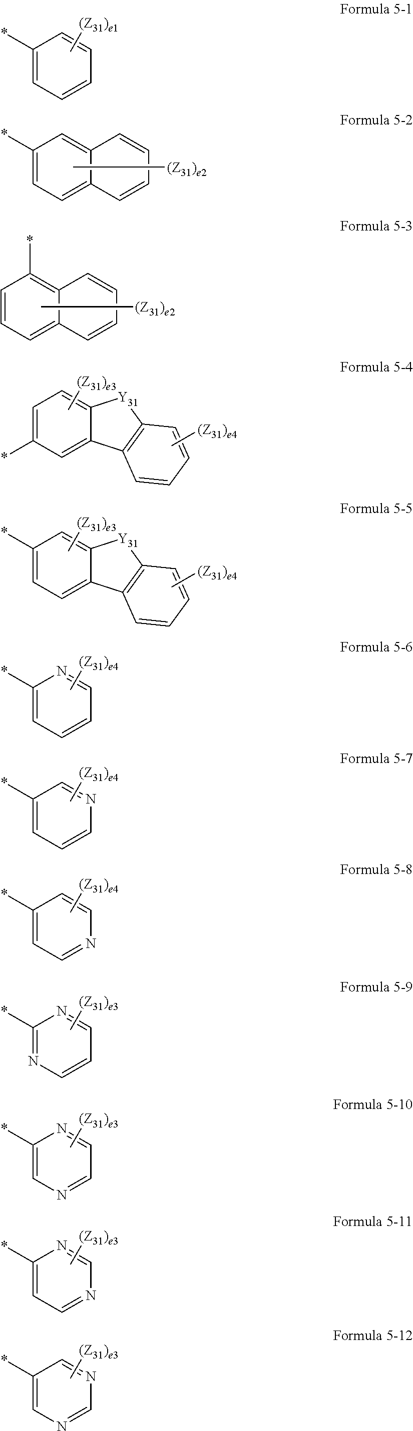

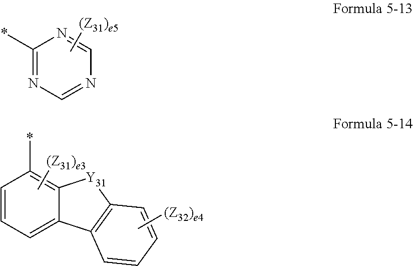

In Formulae 5-1 to 5-14,

Y₃₁ may be O, S, C(Z₃₃)(Z₃₄), or N(Z₃₅);

Z₃₁ to Z₃₅ may be each independently selected from:

a hydrogen, a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, and a C₁-C₂₀ alkoxy group;

a C₁-C₂₀ alkyl group and a C₁-C₂₀ alkoxy group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, and a phosphoric acid or a salt thereof;

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, a triazinyl group, a dibenzofuranyl group, a dibenzothiophenyl group, a benzocarbazolyl group, and a dibenzocarbazolyl group;

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, a triazinyl group, a dibenzofuranyl group, a dibenzothiophenyl group, a benzocarbazolyl group, and a dibenzocarbazolyl group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₁-C₂₀ alkoxy group, a phenyl group and a naphthyl group; and

—Si(Q₃₁)(Q₃₂)(Q₃₃),

wherein Q₁ to Q₃, Q₈, Q₉, and Q₃₁ to Q₃₃ may be each independently selected from a hydrogen, a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₁-C₂₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group; and

e1 may be an integer selected from 0 to 5, e2 may be an integer selected from 1 to 7, e3 may be an integer selected from 1 to 3, e4 may be an integer selected from 1 to 4, e5 may be an integer of 1 or 2, and * is a binding site to a neighboring atom.

According to some embodiments, in Formula 1, R₁ and R₂ may be each independently a group represented by one of *—Si(Me)₃, *—Si(Ph)₃, *—CH═NH, and Formulae 6-1 to 6-25 below:

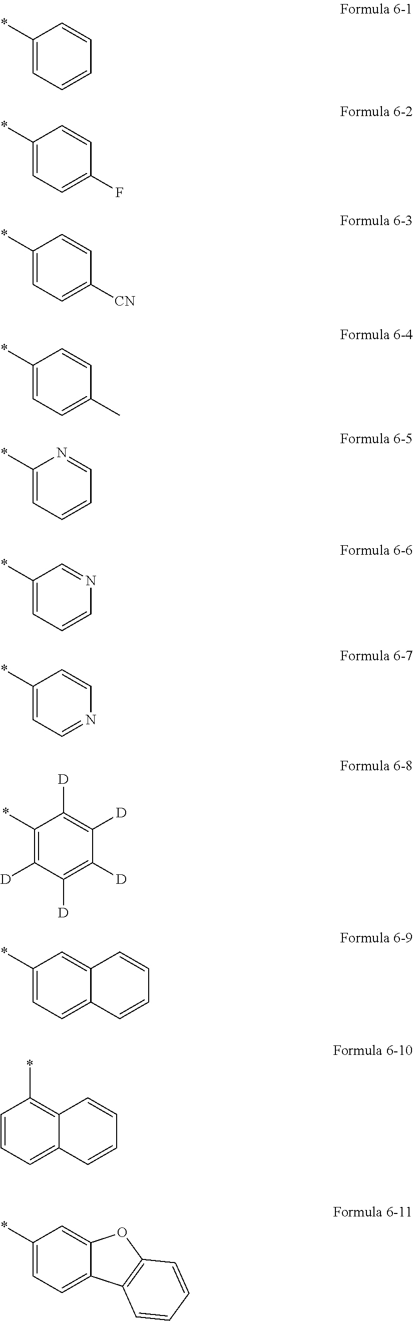

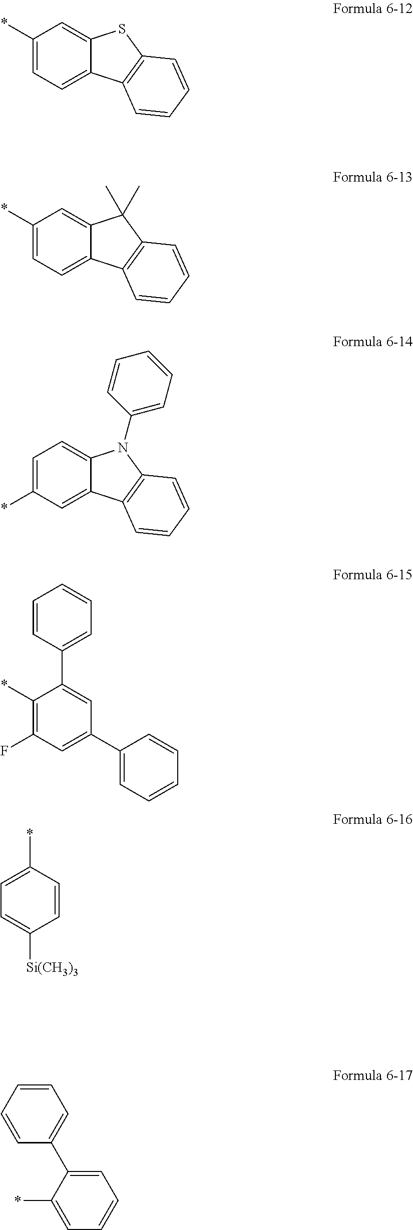

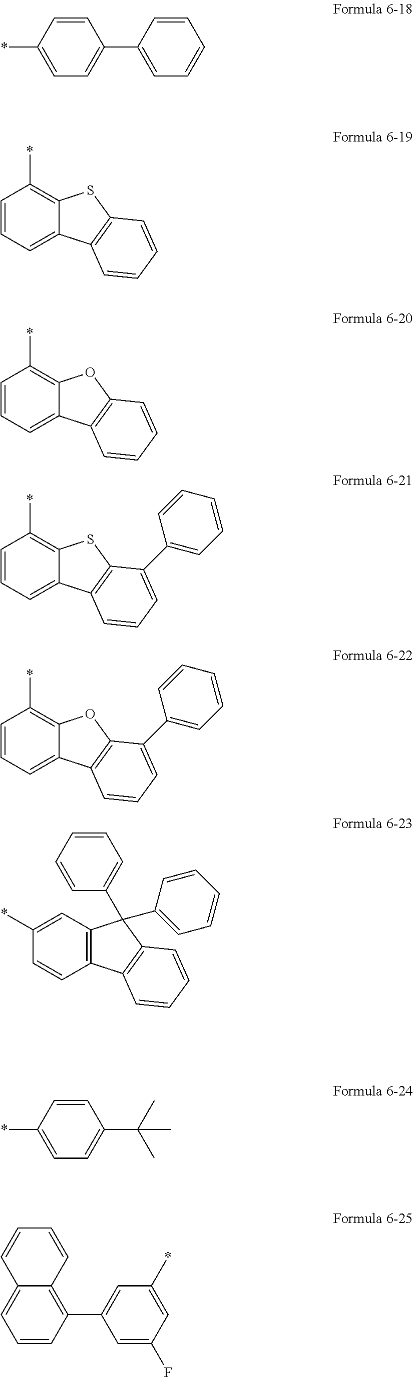

In Formulae 6-1 to 6-25, * is a binding site to a neighboring atom.

In Formula 1, b1 denotes the number of R₁ which may be selected from an integer of 1 to 4. In some embodiments, b1 may be 1 or 2. When b1 is 2 or greater, a plurality of R₁ may be identical to or different from each other. Description of b2 is as defined the same in connection with the description of b1 and a structure of Formula 1.

In Formula 1, R₁₁ to R₂₀ may be each independently selected from:

a hydrogen, a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted heteroaryl group, a substituted or unsubstituted monovalent non-aromatic condensed polycyclic group, a substituted or unsubstituted monovalent non-aromatic condensed heteropolycyclic group, —Si(Q₁)(Q₂)(Q₃), —B(Q₄)(Q₅), and —N(Q₆)(Q₇).

For example, in Formula 1, R₁₁ to R₂₀ may be each independently selected from a hydrogen, a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a substituted or unsubstituted C₁-C₂₀ alkyl group, a substituted or unsubstituted C₁-C₂₀ alkoxy group, a substituted or unsubstituted C₆-C₂₀ aryl group, a substituted or unsubstituted heteroaryl group, a substituted or unsubstituted monovalent non-aromatic condensed polycyclic group, a substituted or unsubstituted monovalent non-aromatic condensed heteropolycyclic group, and —Si(Q₁)(Q₂)(Q₃).

For example, in Formula 1, R₁₁ to R₂₀ may be each independently selected from a hydrogen, a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a substituted or unsubstituted C₁-C₂₀ alkyl group, and a substituted or unsubstituted C₁-C₂₀ alkoxy group.

According to an implementation, the condensed cyclic compound represented by Formula 1 may be represented by one of Formulae 1-1 to 1-4:

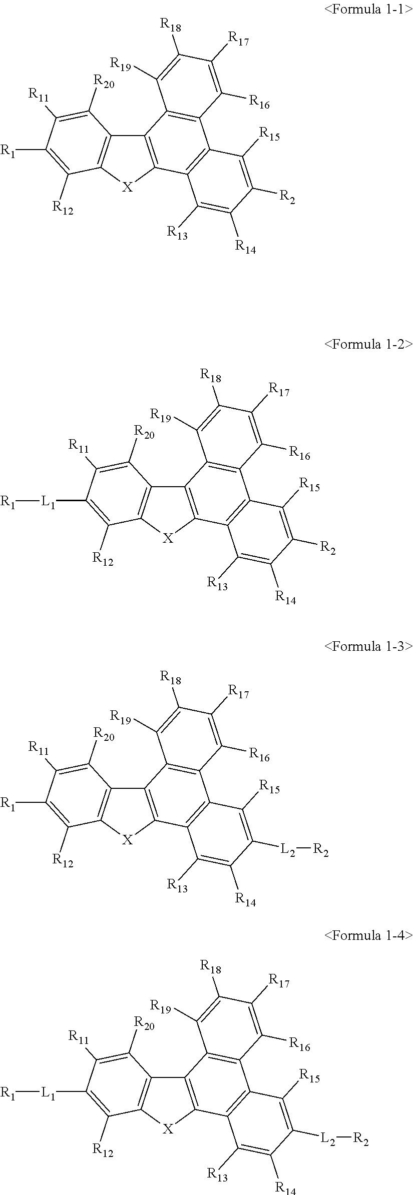

In Formulae 1-1 to 1-4, X, L₁, L₂, R₁, R₂, and R₁₁ to R₂₀ may be understood by referring to the description provided herein with respect to Formula 1.

For example, in Formulae 1-1 to 1-4, L₁ and L₂ may be each independently represented by one of Formulae 4-1 to 4-23 above.

According to some implementations, in Formulae 1-1 to 1-4, R₁ and R₂ may be each independently a group represented by one of *—Si(Me)₃, *—Si(Ph)₃, *—CH═NH, and Formulae 6-1 to 6-25 above.

According to some implementations, in Formulae 1-1 to 1-4, R₁₁ to R₂₀ may be each independently selected from a hydrogen, a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a substituted or unsubstituted C₁-C₂₀ alkyl group, and a substituted or unsubstituted C₁-C₂₀ alkoxy group.

The condensed cyclic compound represented by Formula 1 may be one of Compounds 1 to 46:

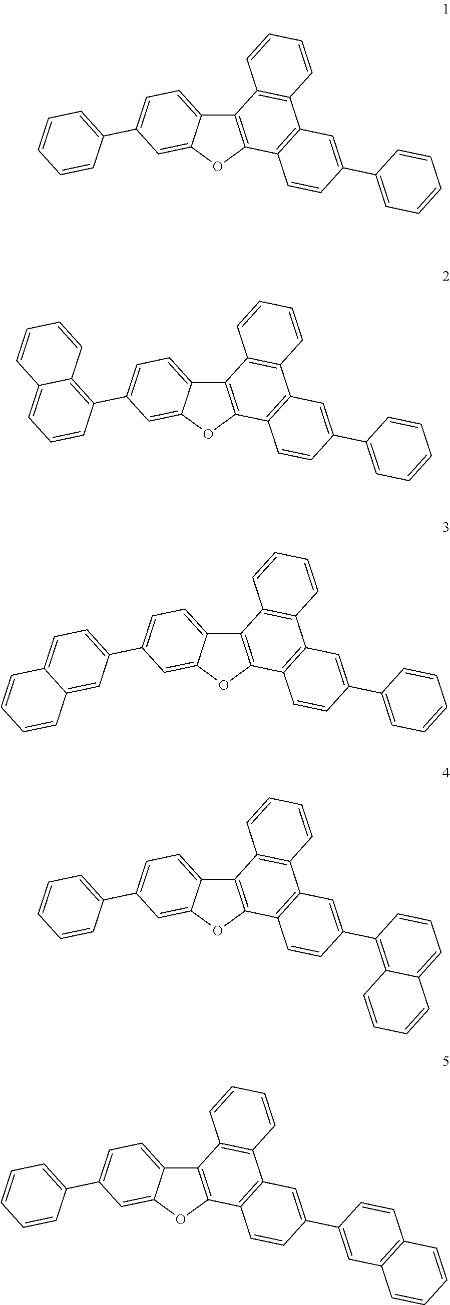 [SMILES: c1ccc(-c2ccc3c(c2)oc2c4ccc(-c5ccccc5)cc4c4ccccc4c32)cc1 ; c1ccc(-c2ccc3c(c2)c2ccccc2c2c4ccc(-c5cccc6ccccc56)cc4oc32)cc1 ; c1ccc(-c2ccc3c(c2)c2ccccc2c2c4ccc(-c5ccc6ccccc6c5)cc4oc32)cc1 ; c1ccc(-c2ccc3c(c2)oc2c4ccc(-c5cccc6ccccc56)cc4c4ccccc4c32)cc1 ; c1ccc(-c2ccc3c(c2)oc2c4ccc(-c5ccc6ccccc6c5)cc4c4ccccc4c32)cc1]

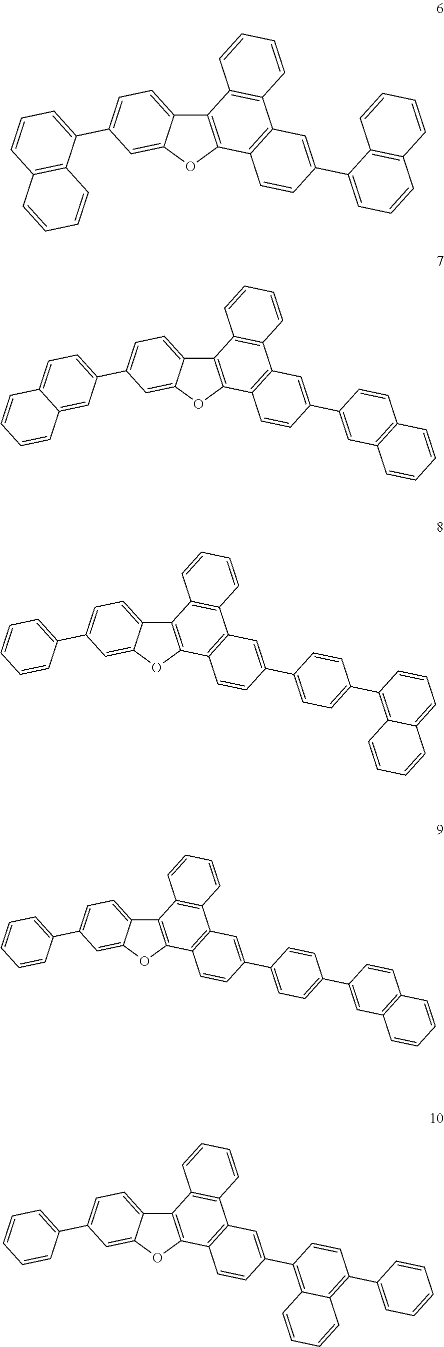 [SMILES: c1ccc2c(-c3ccc4c(c3)oc3c5ccc(-c6cccc7ccccc67)cc5c5ccccc5c43)cccc2c1 ; c1ccc2cc(-c3ccc4c(c3)oc3c5ccc(-c6ccc7ccccc7c6)cc5c5ccccc5c43)ccc2c1 ; c1ccc(-c2ccc3c(c2)oc2c4ccc(-c5ccc(-c6cccc7ccccc67)cc5)cc4c4ccccc4c32)cc1 ; c1ccc(-c2ccc3c(c2)oc2c4ccc(-c5ccc(-c6ccc7ccccc7c6)cc5)cc4c4ccccc4c32)cc1 ; c1ccc(-c2ccc3c(c2)oc2c4ccc(-c5ccc(-c6ccccc6)c6ccccc56)cc4c4ccccc4c32)cc1]

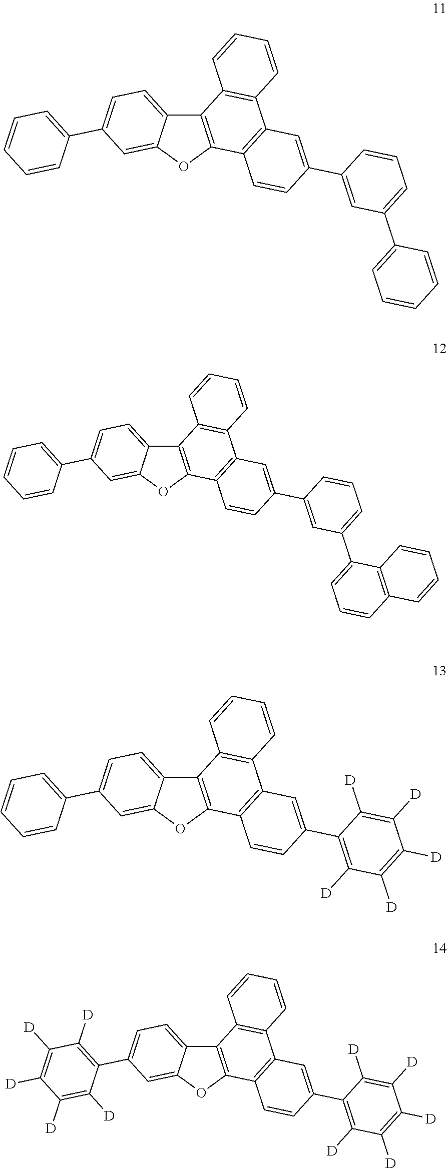 [SMILES: c1ccc(-c2cccc(-c3ccc4c(c3)c3ccccc3c3c5ccc(-c6ccccc6)cc5oc43)c2)cc1 ; c1ccc(-c2ccc3c(c2)oc2c4ccc(-c5cccc(-c6cccc7ccccc67)c5)cc4c4ccccc4c32)cc1 ; [2H]c1c([2H])c([2H])c(-c2ccc3c(c2)c2ccccc2c2c4ccc(-c5ccccc5)cc4oc32)c([2H])c1[2H] ; [2H]c1c([2H])c([2H])c(-c2ccc3c(c2)oc2c4ccc(-c5c([2H])c([2H])c([2H])c([2H])c5[2H])cc4c4ccccc4c32)c([2H])c1[2H]]

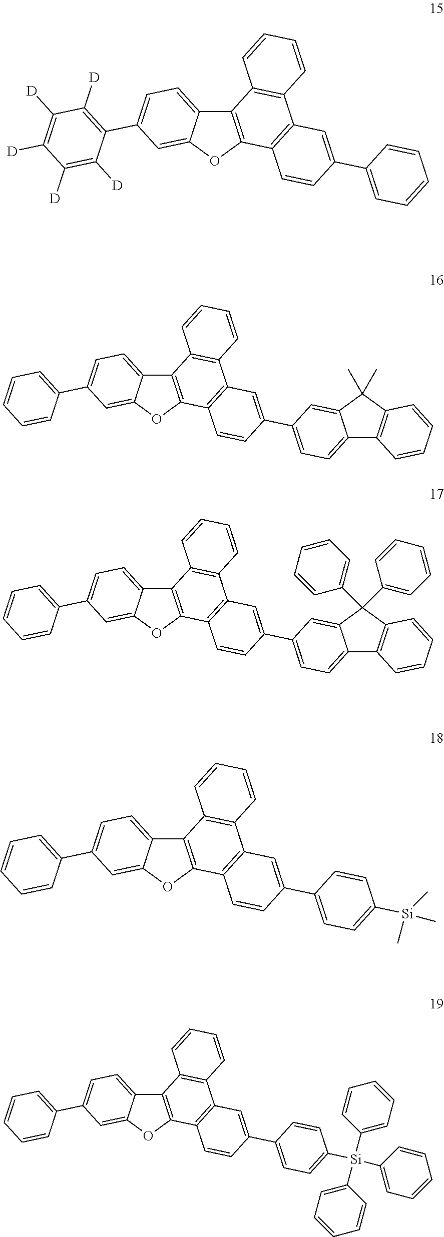 [SMILES: [2H]c1c([2H])c([2H])c(-c2ccc3c(c2)oc2c4ccc(-c5ccccc5)cc4c4ccccc4c32)c([2H])c1[2H] ; CC1(C)c2ccccc2-c2ccc(-c3ccc4c(c3)c3ccccc3c3c5ccc(-c6ccccc6)cc5oc43)cc21 ; c1ccc(-c2ccc3c(c2)oc2c4ccc(-c5ccc6c(c5)C(c5ccccc5)(c5ccccc5)c5ccccc5-6)cc4c4ccccc4c32)cc1 ; C[Si](C)(C)c1ccc(-c2ccc3c(c2)c2ccccc2c2c4ccc(-c5ccccc5)cc4oc32)cc1 ; c1ccc(-c2ccc3c(c2)oc2c4ccc(-c5ccc([Si](c6ccccc6)(c6ccccc6)c6ccccc6)cc5)cc4c4ccccc4c32)cc1]

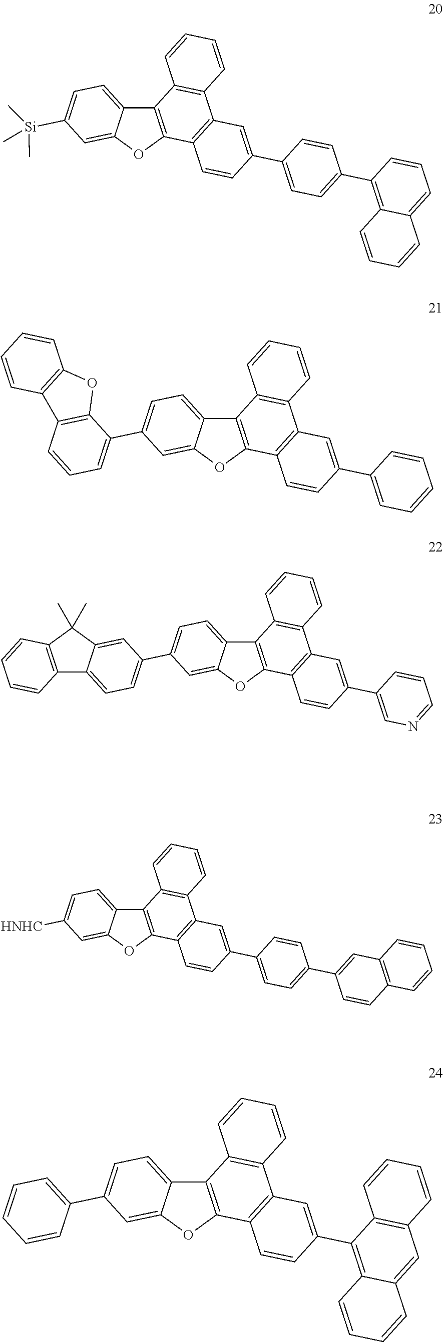

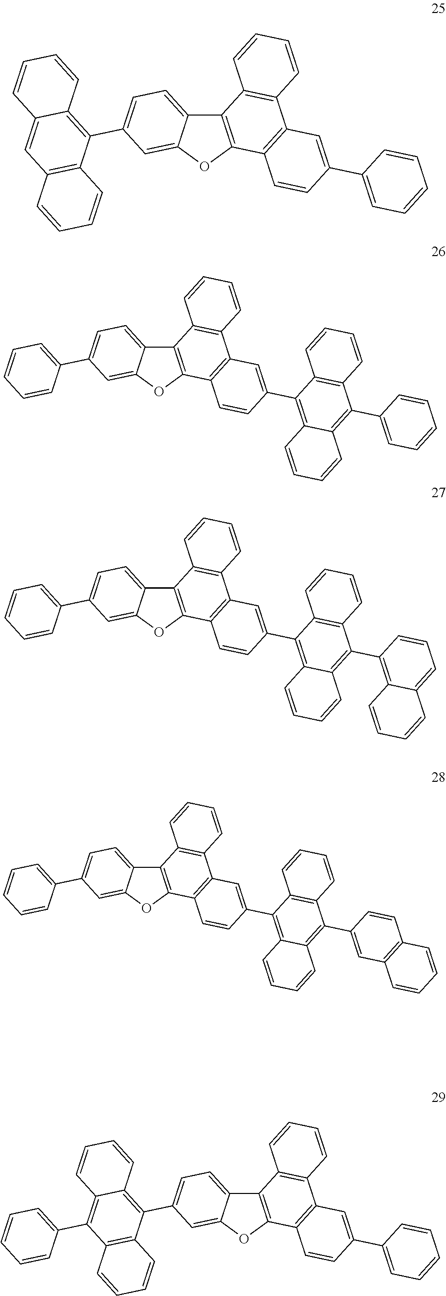 [SMILES: c1ccc(-c2ccc3c(c2)c2ccccc2c2c4ccc(-c5c6ccccc6cc6ccccc56)cc4oc32)cc1 ; c1ccc(-c2ccc3c(c2)oc2c4ccc(-c5c6ccccc6c(-c6ccccc6)c6ccccc56)cc4c4ccccc4c32)cc1 ; c1ccc(-c2ccc3c(c2)oc2c4ccc(-c5c6ccccc6c(-c6cccc7ccccc67)c6ccccc56)cc4c4ccccc4c32)cc1 ; c1ccc(-c2ccc3c(c2)oc2c4ccc(-c5c6ccccc6c(-c6ccc7ccccc7c6)c6ccccc56)cc4c4ccccc4c32)cc1 ; c1ccc(-c2ccc3c(c2)c2ccccc2c2c4ccc(-c5c6ccccc6c(-c6ccccc6)c6ccccc56)cc4oc32)cc1]

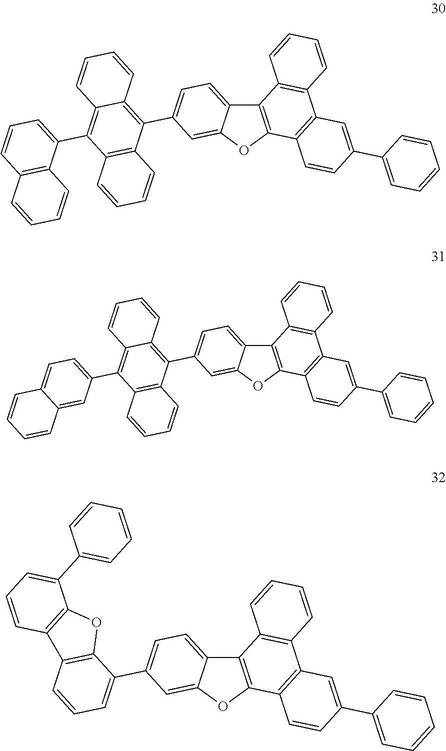 [SMILES: c1ccc(-c2ccc3c(c2)c2ccccc2c2c4ccc(-c5c6ccccc6c(-c6cccc7ccccc67)c6ccccc56)cc4oc32)cc1 ; c1ccc(-c2ccc3c(c2)c2ccccc2c2c4ccc(-c5c6ccccc6c(-c6ccc7ccccc7c6)c6ccccc56)cc4oc32)cc1 ; c1ccc(-c2ccc3c(c2)c2ccccc2c2c4ccc(-c5cccc6c5oc5c(-c7ccccc7)cccc56)cc4oc32)cc1]

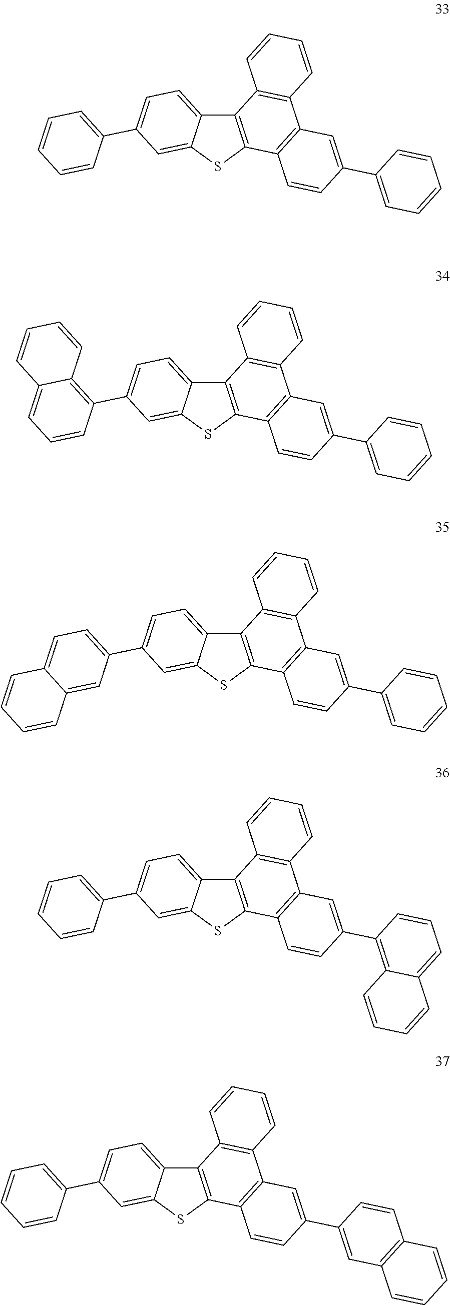 [SMILES: c1ccc(-c2ccc3c(c2)sc2c4ccc(-c5ccccc5)cc4c4ccccc4c32)cc1 ; c1ccc(-c2ccc3c(c2)c2ccccc2c2c4ccc(-c5cccc6ccccc56)cc4sc32)cc1 ; c1ccc(-c2ccc3c(c2)c2ccccc2c2c4ccc(-c5ccc6ccccc6c5)cc4sc32)cc1 ; c1ccc(-c2ccc3c(c2)sc2c4ccc(-c5cccc6ccccc56)cc4c4ccccc4c32)cc1 ; c1ccc(-c2ccc3c(c2)sc2c4ccc(-c5ccc6ccccc6c5)cc4c4ccccc4c32)cc1]

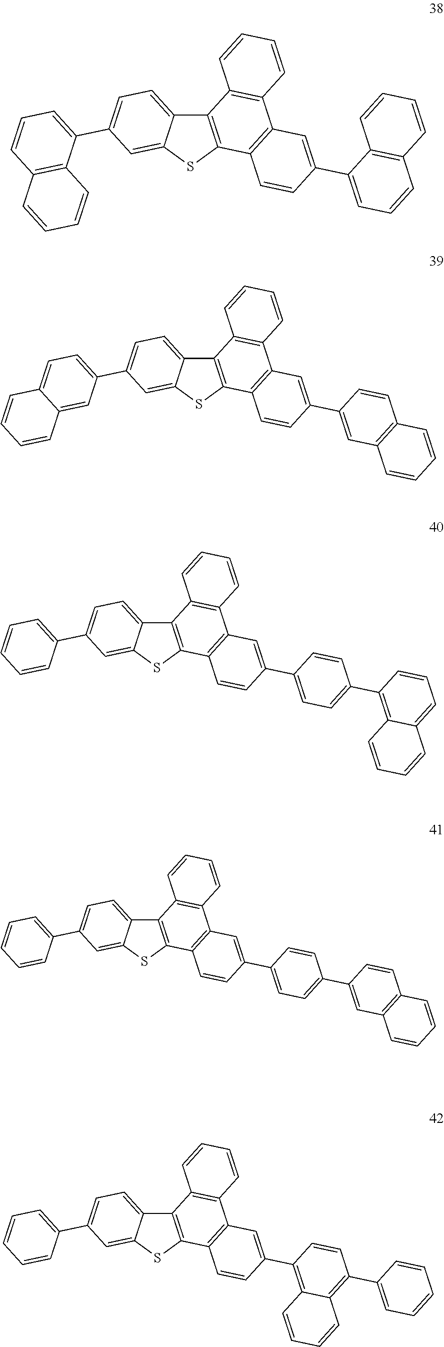 [SMILES: c1ccc2c(-c3ccc4c(c3)sc3c5ccc(-c6cccc7ccccc67)cc5c5ccccc5c43)cccc2c1 ; c1ccc2cc(-c3ccc4c(c3)sc3c5ccc(-c6ccc7ccccc7c6)cc5c5ccccc5c43)ccc2c1 ; c1ccc(-c2ccc3c(c2)sc2c4ccc(-c5ccc(-c6cccc7ccccc67)cc5)cc4c4ccccc4c32)cc1 ; c1ccc(-c2ccc3c(c2)sc2c4ccc(-c5ccc(-c6ccc7ccccc7c6)cc5)cc4c4ccccc4c32)cc1 ; c1ccc(-c2ccc3c(c2)sc2c4ccc(-c5ccc(-c6ccccc6)c6ccccc56)cc4c4ccccc4c32)cc1]

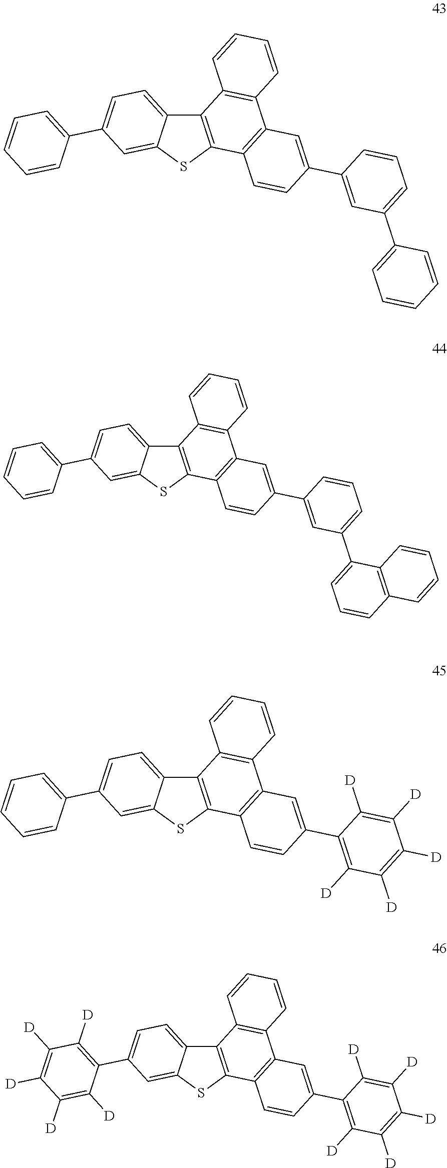 [SMILES: c1ccc(-c2cccc(-c3ccc4c(c3)c3ccccc3c3c5ccc(-c6ccccc6)cc5sc43)c2)cc1 ; c1ccc(-c2ccc3c(c2)sc2c4ccc(-c5cccc(-c6cccc7ccccc67)c5)cc4c4ccccc4c32)cc1 ; [2H]c1c([2H])c([2H])c(-c2ccc3c(c2)c2ccccc2c2c4ccc(-c5ccccc5)cc4sc32)c([2H])c1[2H] ; [2H]c1c([2H])c([2H])c(-c2ccc3c(c2)sc2c4ccc(-c5c([2H])c([2H])c([2H])c([2H])c5[2H])cc4c4ccccc4c32)c([2H])c1[2H]]

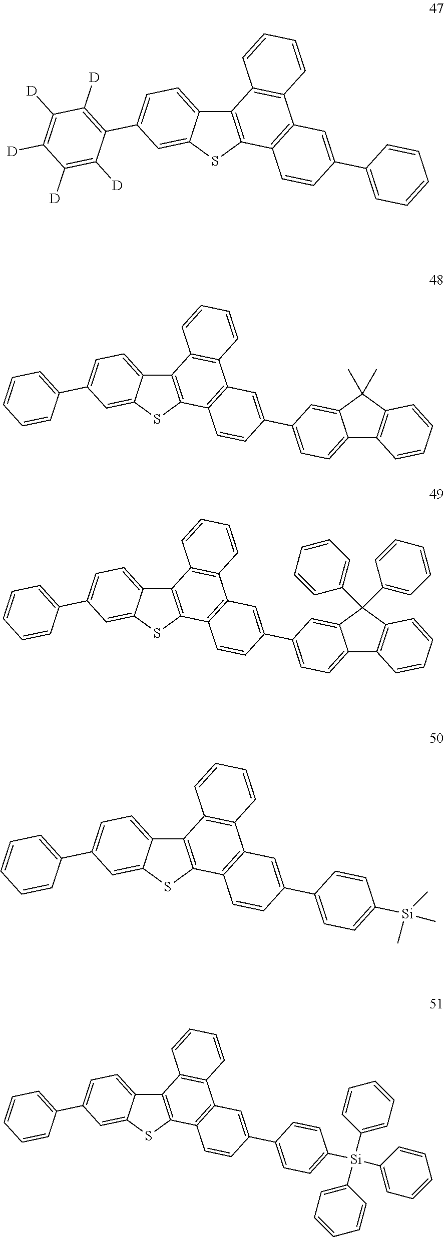 [SMILES: [2H]c1c([2H])c([2H])c(-c2ccc3c(c2)sc2c4ccc(-c5ccccc5)cc4c4ccccc4c32)c([2H])c1[2H] ; CC1(C)c2ccccc2-c2ccc(-c3ccc4c(c3)c3ccccc3c3c5ccc(-c6ccccc6)cc5sc43)cc21 ; c1ccc(-c2ccc3c(c2)sc2c4ccc(-c5ccc6c(c5)C(c5ccccc5)(c5ccccc5)c5ccccc5-6)cc4c4ccccc4c32)cc1 ; C[Si](C)(C)c1ccc(-c2ccc3c(c2)c2ccccc2c2c4ccc(-c5ccccc5)cc4sc32)cc1 ; c1ccc(-c2ccc3c(c2)sc2c4ccc(-c5ccc([Si](c6ccccc6)(c6ccccc6)c6ccccc6)cc5)cc4c4ccccc4c32)cc1]

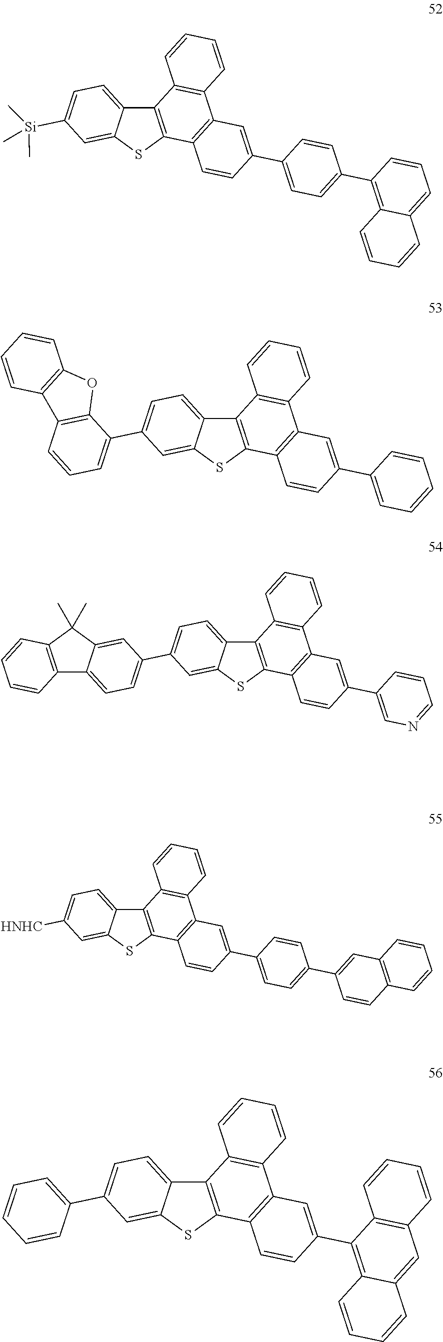

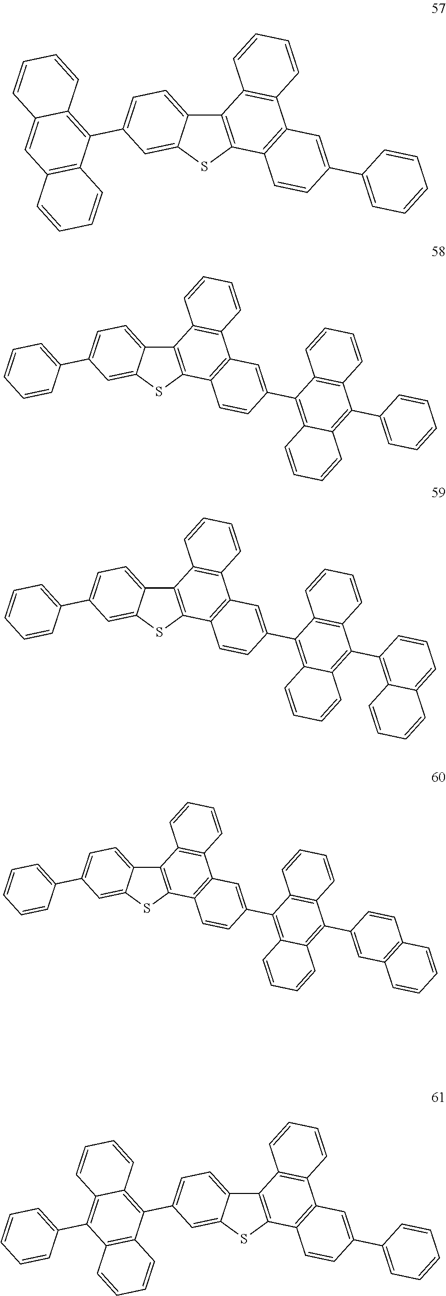 [SMILES: c1ccc(-c2ccc3c(c2)c2ccccc2c2c4ccc(-c5c6ccccc6cc6ccccc56)cc4sc32)cc1 ; c1ccc(-c2ccc3c(c2)sc2c4ccc(-c5c6ccccc6c(-c6ccccc6)c6ccccc56)cc4c4ccccc4c32)cc1 ; c1ccc(-c2ccc3c(c2)sc2c4ccc(-c5c6ccccc6c(-c6cccc7ccccc67)c6ccccc56)cc4c4ccccc4c32)cc1 ; c1ccc(-c2ccc3c(c2)sc2c4ccc(-c5c6ccccc6c(-c6ccc7ccccc7c6)c6ccccc56)cc4c4ccccc4c32)cc1 ; c1ccc(-c2ccc3c(c2)c2ccccc2c2c4ccc(-c5c6ccccc6c(-c6ccccc6)c6ccccc56)cc4sc32)cc1]

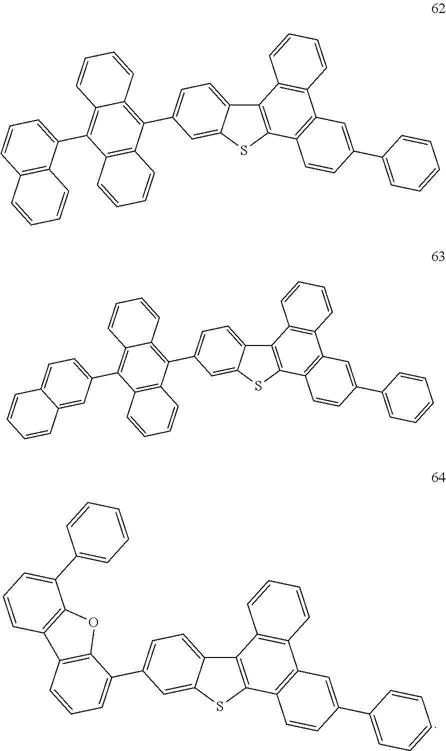 [SMILES: c1ccc(-c2ccc3c(c2)c2ccccc2c2c4ccc(-c5c6ccccc6c(-c6cccc7ccccc67)c6ccccc56)cc4sc32)cc1 ; c1ccc(-c2ccc3c(c2)c2ccccc2c2c4ccc(-c5c6ccccc6c(-c6ccc7ccccc7c6)c6ccccc56)cc4sc32)cc1 ; c1ccc(-c2ccc3c(c2)c2ccccc2c2c4ccc(-c5cccc6c5oc5c(-c7ccccc7)cccc56)cc4sc32)cc1]

The condensed cyclic compound represented by Formula 1 may include a core that is represented by Formula 1′ or Formula 1″ in which a ring including an electron-rich heteroatom, such as an oxygen atom or a sulfur atom, and a phenanthrene moiety are bonded to each other. Thus a dipole in the molecule may increase. When the condensed cyclic compound is used as an organic light-emitting material, a quantum efficiency during electron excitation and light-emission may increase.

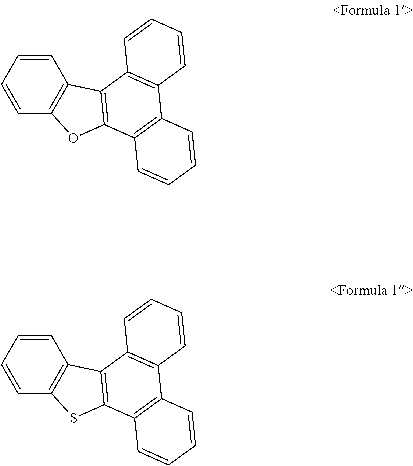 [SMILES: c1ccc2c(c1)oc1c3ccccc3c3ccccc3c21 ; c1ccc2c(c1)sc1c3ccccc3c3ccccc3c21]

In Formula 1, b1 and b2 may each independently be an integer selected from 1 to 4, and R₁ and R₂ are not a hydrogen. The core represented by Formula 1′ or Formula 1″ is necessarily substituted with R₁ and R₂, not a hydrogen. Thus, an electron density at the core may increase. An organic light-emitting device including the condensed cyclic compound represented by Formula 1 may have excellent light-emitting characteristics.

The condensed cyclic compound represented by Formula 1 may be synthesized by using a suitable organic synthetic method. A method of synthesizing the condensed cyclic compound may be understood by one of ordinary skill in the art with reference to examples described herein.

The condensed cyclic compound represented by Formula 1 may be included between a pair of electrodes in an organic light-emitting device. For example, the condensed cyclic compound may be included in an electron transport region or in an electron transport layer. In this regard, an organic light-emitting device may include a first electrode, a second electrode facing the first electrode, and an organic layer that is disposed between the first electrode and the second electrode and includes an emission layer. The organic layer includes the condensed cyclic compound represented by Formula 1.

As used herein, the expression “the (organic layer) includes at least one condensed cyclic compounds” may be construed as “the (organic layer) may include one condensed cyclic compound represented by Formula 1 or two or more different condensed cyclic compounds represented by Formula 1”.

In one implementation, an organic layer may include only Compound 1 as the condensed cyclic compound. For example, Compound 1 may be included in an emission layer of an organic light-emitting device. In another implementation, an organic layer may include Compound 1 and Compound 2 as the condensed cyclic compound. For example, Compound 1 and Compound 2 may be included in the same layer (for example, Compound 1 and Compound 2 may all be included in an emission layer) or in different layers (for example, Compound 1 may be included in an emission layer and Compound 2 may be included in an electron transport layer).

The organic layer may include, for example, i) a hole-transport region that is disposed between the first electrode (an anode) and the emission layer and includes at least one selected from a hole injection layer, a hole transport layer, a buffer layer, and an electron blocking layer; and ii) an electron transport region that is disposed between the emission layer and the second electrode (a cathode) and includes at least one selected from a hole blocking layer, an electron transport layer, and an electron injection layer. The emission layer may include the condensed cyclic compound represented by Formula 1.

As used herein, the term the “organic layer” may refer to a single layer and/or to a plurality of layers disposed between the first electrode and the second electrode in an organic light-emitting device. A material included in the “organic layer” may include other materials besides an organic material.

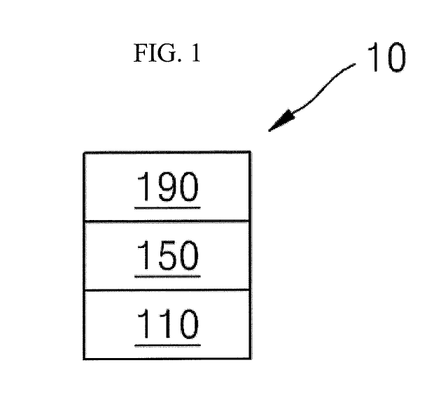

FIG. 1 illustrates a schematic view of an organic light-emitting device 10 according to an exemplary embodiment. The organic light-emitting device 10 may include a first electrode 110, an organic layer 150, and a second electrode 190.

Hereinafter, a structure of the organic light-emitting device 10 and a method of manufacturing the organic light-emitting device 10 will be described with reference to FIG. 1.

As shown in FIG. 1, a substrate may be additionally disposed under the first electrode 110 or on the second electrode 190. The substrate may be a glass substrate or a transparent plastic substrate, each having excellent mechanical strength, thermal stability, transparency, surface smoothness, ease of handling, and water repellency.

For example, the first electrode 110 may be formed by depositing or sputtering a first electrode material on the substrate. When the first electrode 110 is an anode, the first electrode material may be selected from materials with a high work function for easy hole injection. The first electrode 110 may be a reflective electrode, a semi-transmissive electrode, or a transmissive electrode. The first electrode material may be an indium tin oxide (ITO), an indium zinc oxide (IZO), a tin oxide (SnO₂), or a zinc oxide (ZnO). In some embodiments, the first electrode material may be at least one selected from magnesium (Mg), aluminum (Al), aluminum-lithium (Al—Li), calcium (Ca), magnesium-indium (Mg—In), and magnesium-silver (Mg—Ag).

The first electrode 110 may have a single-layer structure or may be a multi-layer structure including two or more layers. For example, the first electrode 110 may have a triple-layer structure of ITO/Ag/ITO.

The organic layer 150 may be disposed on the first electrode 110. The organic layer 150 may include an emission layer.

The organic layer 150 may further include a hole transport region that is disposed between the first electrode and the emission layer and an electron transport region that is disposed between the emission layer and the second electrode.

The hole transport region may include at least one of a hole injection layer (HIL), a hole transport layer (HTL), a buffer layer, and an electron blocking layer (EBL). The electron transport region may include at least one of a hole blocking layer (HBL), an electron transport layer (ETL), and an electron injection layer (EIL).

The hole transport region may have a single-layered structure formed of a single material, a single-layered structure formed of a plurality of different materials, or a multi-layered structure having a plurality of layers formed of a plurality of different materials.

For example, the hole transport region may have a single-layered structure formed of a plurality of different materials, or a structure of hole injection layer/hole transport layer, a structure of hole injection layer/hole transport layer/buffer layer, a structure of hole injection layer/buffer layer, a structure of hole transport layer/buffer layer, or a structure of hole injection layer/hole transport layer/electron blocking layer.

When the hole transport region includes a hole injection layer, the hole injection layer may be formed on the first electrode 110 by using various methods, such as vacuum deposition, spin coating, casting, a Langmuir-Blodgett (LB) method, ink-jet printing, laser-printing, or laser-induced thermal imaging (LITI).

When a hole injection layer is formed by vacuum deposition, for example, the vacuum deposition may be performed at a deposition temperature in a range of about 100° C. to about 500° C., at a vacuum degree in a range of about 10⁻⁸ torr to about 10⁻³ torr, and at a deposition rate in a range of about 0.01 Å/sec to about 100 Å/sec, in consideration of a compound for forming a hole injection layer and a structure of a desired hole injection layer.

When a hole injection layer is formed by spin coating, the spin coating may be performed at a coating rate in a range of about 2,000 rpm to about 5,000 rpm and at a temperature in a range of about 80° C. to about 200° C., in consideration of a compound for forming a hole injection layer and a structure of a desired hole injection layer.

When the hole transport region includes a hole transport layer, the hole transport layer may be formed on the first electrode 110 or on the hole injection layer by a suitable method, such as vacuum deposition, spin coating, casting, a LB method, ink-jet printing, laser-printing, or LITI. When the hole transport layer is formed by vacuum deposition or by spin coating, the deposition conditions or the coating conditions may be inferred based on the conditions for forming the hole injection layer.

The hole transport region may include, for example, at least one of m-MTDATA, TDATA, 2-TNATA, NPB, β-NPB, TPD, Spiro-TPD, Spiro-NPB, α-NPB, TAPC, HMTPD, 4,4′,4″-tris(N-carbazolyl)triphenylamine (TCTA), polyaniline/dodecyla benzenesulfonic acid (Pani/DBSA), poly(3,4-ethylenedioxythiophene)/poly(4-styrenesulfonate) (PEDOT/PSS), polyaniline/camphor sulfonicacid (Pani/CSA), (polyaniline)/poly(4-styrenesulfonate) (PANI/PSS), a compound represented by Formula 201, or a compound represented by Formula 202:

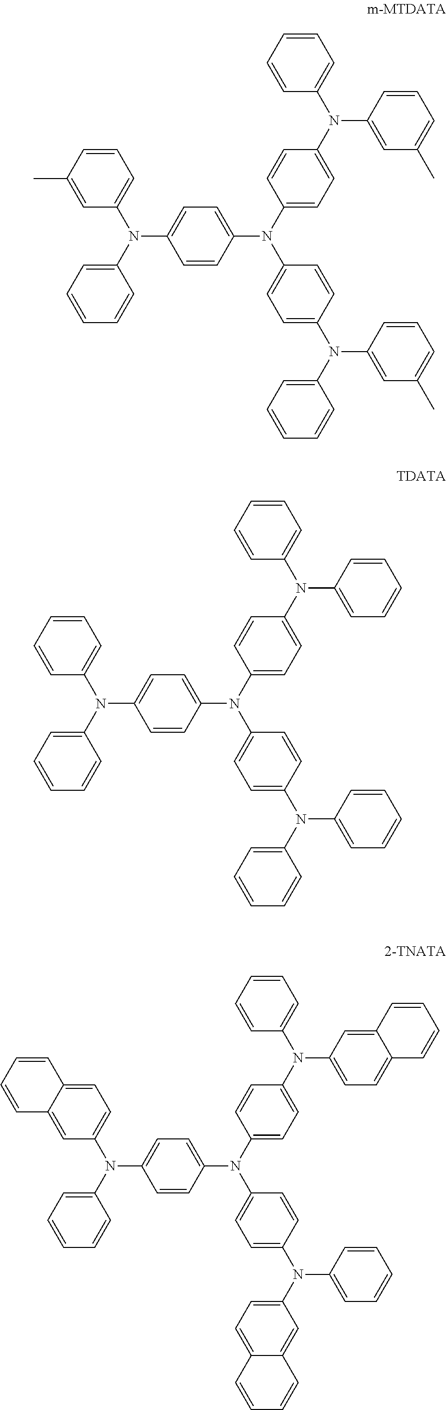

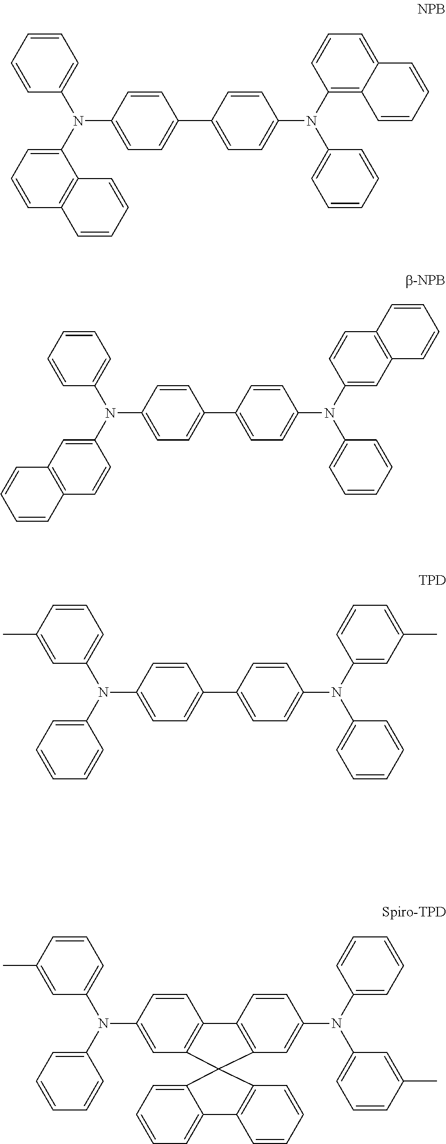

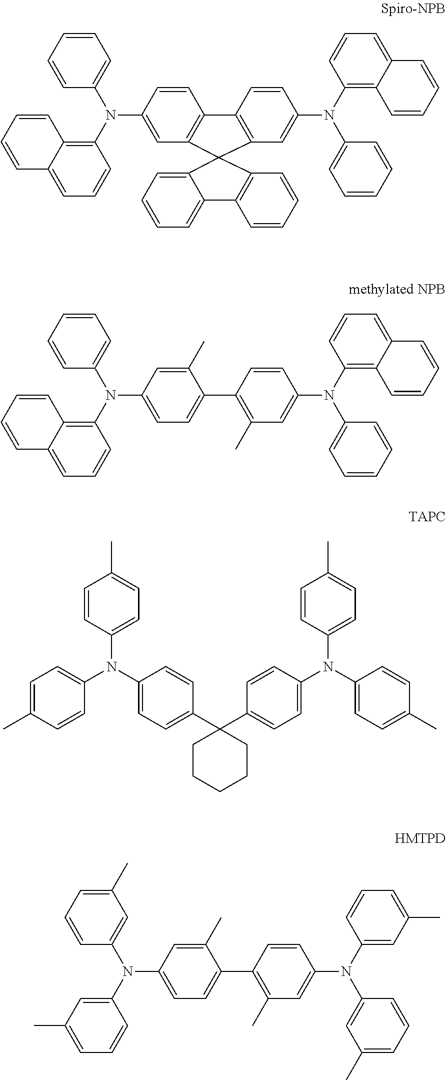

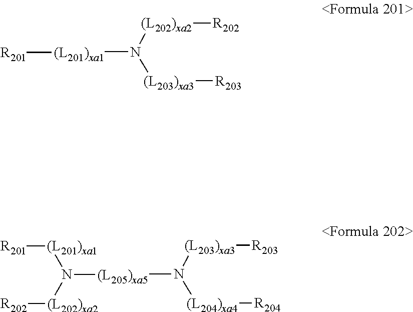

In Formulae 201 and 202,

L₂₀₁ to L₂₀₅ may have the same definition as L₁ and L₂ in Formula 1;

xa1 to xa4 may be each independently selected from 0, 1, 2, and 3;

xa5 may be selected from 1, 2, 3, 4, and 5;

R₂₀₁ to R₂₀₄ may be each independently selected from a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group.

For example, in Formulae 201 and 202,

L₂₀₁ to L₂₀₅ may be each independently selected from:

a phenylene group, a naphthylene group, a fluorenylene group, a spiro-fluorenylene group, a benzofluorene group, a dibenzofluorene group, a phenanthrenylene group, an anthracenylene group, a pyrenylene group, a chrysenylene group, a pyridinylene group, a pyrazinylene group, a pyrimidinylene group, a pyridazinylene group, a quinolinylene group, an isoquinolinylene group, a quinoxalinylene group, a quinazolinylene group, a carbazolylene group, and a triazinylene group; and

a phenylene group, a naphthylene group, a fluorenylene group, a spiro-fluorenylene group, a benzofluorenylene group, a dibenzofluorenylene group, a phenanthrenylene group, an anthracenylene group, a pyrenylene group, a chrysenylene group, a pyridinylene group, a pyrazinylene group, a pyrimidinylene group, a pyridazinylene group, a quinolinylene group, an isoquinolinylene group, a quinoxalinylene group, a quinazolinylene group, a carbazolylene group, and a triazinylene group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₁-C₂₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, an isoindolyl group, a quinolinyl group, an isoquinolinyl group, a quinoxalinyl group, a quinazolinyl group, a carbazolyl group, and a triazinyl group;

xa1 to xa4 may be each independently 0, 1, or 2;

xa5 is 1, 2, or 3; and

R₂₀₁ to R₂₀₄ may be each independently selected from:

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group; and

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₁-C₂₀ alkoxy group, a phenyl group, a naphthyl group, an azulenyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group.

The compound represented by Formula 201 may be represented by Formula 201A:

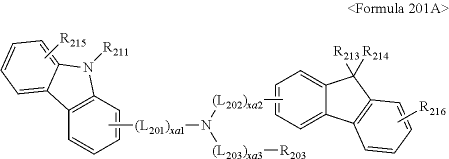

For example, the compound represented by Formula 201 may be represented by Formula 201A-1:

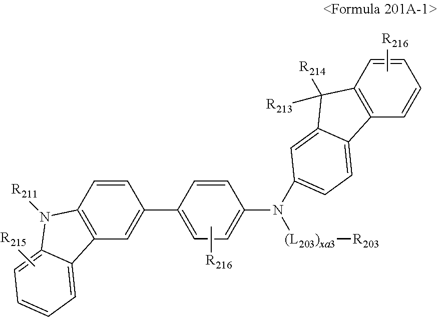

For example, the compound represented by Formula 202 may be represented by Formula 202A:

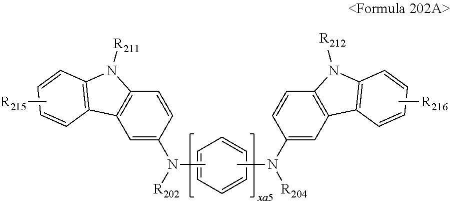

In Formulae 201A, 201A-1, and 202A, description of L₂₀₁ to L₂₀₃, xa1 to xa3, xa5, and R₂₀₂ to R₂₀₄ is the same as defined with respect to Formulae 201 and 202. R₂₁₁ may have the same definition as R₂₀₃ in Formulae 201 and 202. R₂₁₃ to R₂₁₆ may be each independently selected from a hydrogen, a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group.

For example, in Formulae 201A, 201A-1, and 202A,

L₂₀₁ to L₂₀₃ may be each independently selected from:

a phenylene group, a naphthylene group, a fluorenylene group, a spiro-fluorenylene group, a benzofluorenylene group, a dibenzofluorenylene group, a phenanthrenylene group, an anthracenylene group, a pyrenylene group, a chrysenylene group, a pyridinylene group, a pyrazinylene group, a pyrimidinylene group, a pyridazinylene group, a quinolinylene group, an isoquinolinylene group, a quinoxalinylene group, a quinazolinylene group, a carbazolylene group, and a triazinylene group; and

a phenylene group, a naphthylene group, a fluorenylene group, a spiro-fluorenylene group, a benzofluorenylene group, a dibenzofluorenylene group, a phenanthrenylene group, an anthracenylene group, a pyrenylene group, a chrysenylene group, a pyridinylene group, a pyrazinylene group, a pyrimidinylene group, a pyridazinylene group, a quinolinylene group, an isoquinolinylene group, a quinoxalinylene group, a quinazolinylene group, a carbazolylene group, and a triazinylene group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₁-C₂₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group,

xa1 to xa3 may be each independently 0 or 1,

R₂₀₃, R₂₁₁, and R₂₁₂ may be each independently selected from:

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group; and

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group, each substituted with at least one selected from a deuterium, —F, —Cl, —Br, —I, a hydroxyl group, a cyano group, a nitro group, an amino group, an amidino group, a hydrazine group, a hydrazone group, carboxylic acid a carboxylic acid or a salt thereof, a sulfonic acid or a salt thereof, a phosphoric acid or a salt thereof, a C₁-C₂₀ alkyl group, a C₁-C₂₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group,

R₂₁₃ and R₂₁₄ may be each independently selected from:

a C₁-C₂₀ alkyl group and a C₁-C₂₀ alkoxy group;

a C₁-C₂₀ alkyl group and a C₁-C₂₀ alkoxy group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group;

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group; and

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₁-C₂₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group,

R₂₁₅ and R₂₁₆ may be each independently selected from:

a hydrogen, a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, and a C₁-C₂₀ alkoxy group;

a C₁-C₂₀ alkyl group and a C₁-C₂₀ alkoxy group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group;

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, and a triazinyl group; and

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₁-C₂₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group, and

xa5 may be 1 or 2.

In Formulae 201A and 201A-1, R₂₁₃ and R₂₁₄ may be linked to each other and form a saturated or unsaturated ring.

The compound represented by Formula 201 and the compound represented by Formula 202 may include Compounds HT1 to HT20:

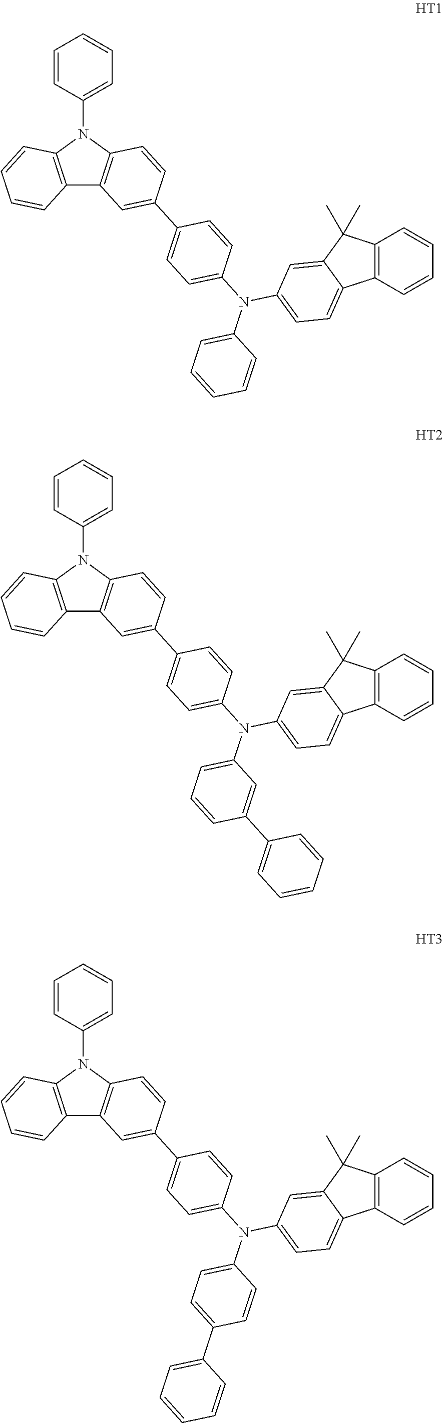 [SMILES: CC1(C)c2ccccc2-c2ccc(N(c3ccccc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21]

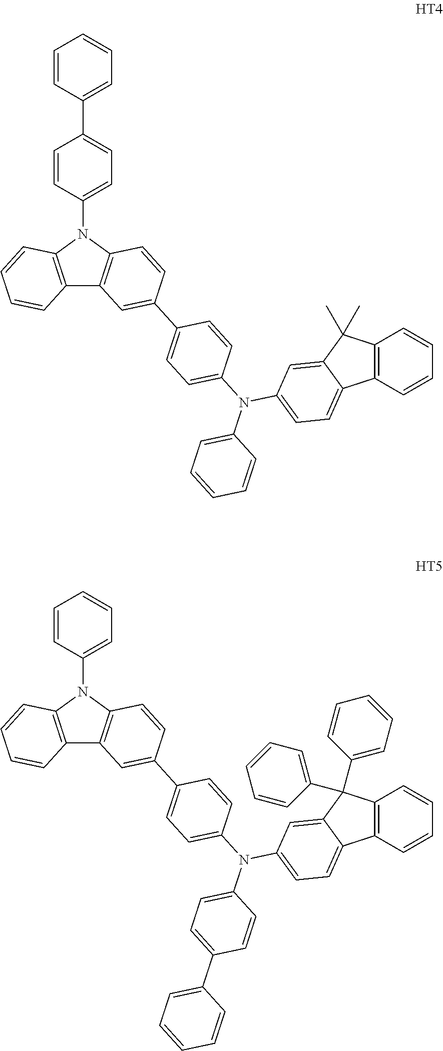 [SMILES: CC1(C)c2ccccc2-c2ccc(N(c3ccccc3)c3ccc(-c4ccc5c(c4)c4ccccc4n5-c4ccc(-c5ccccc5)cc4)cc3)cc21 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

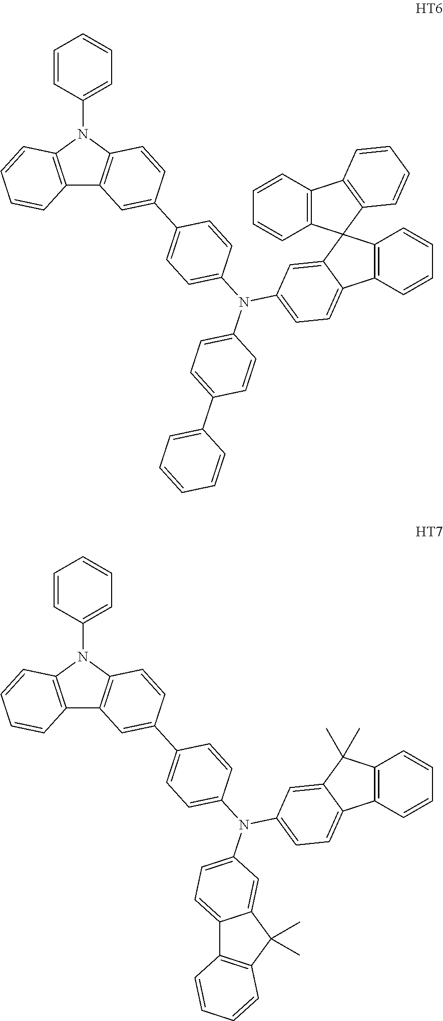 [SMILES: c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1 ; CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21]

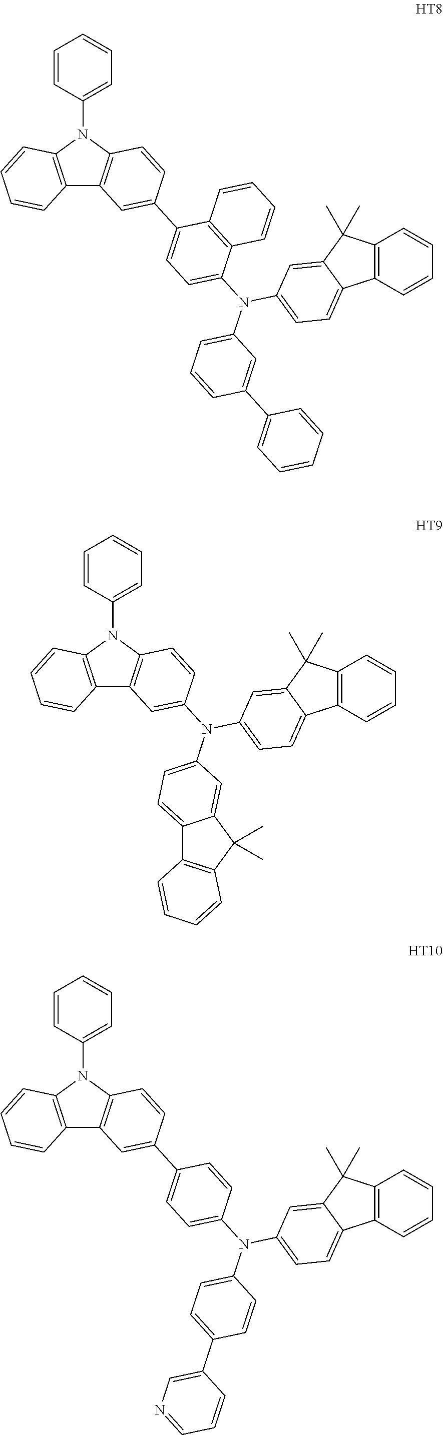 [SMILES: CC1(C)c2ccccc2-c2ccc(N(c3cccc(-c4ccccc4)c3)c3ccc(-c4ccc5c(c4)c4ccccc4n5-c4ccccc4)c4ccccc34)cc21 ; CC1(C)c2ccccc2-c2ccc(N(c3ccc4c(c3)C(C)(C)c3ccccc3-4)c3ccc4c(c3)c3ccccc3n4-c3ccccc3)cc21 ; CC1(C)c2ccccc2-c2ccc(N(c3ccc(-c4cccnc4)cc3)c3ccc(-c4ccc5c(c4)c4ccccc4n5-c4ccccc4)cc3)cc21]

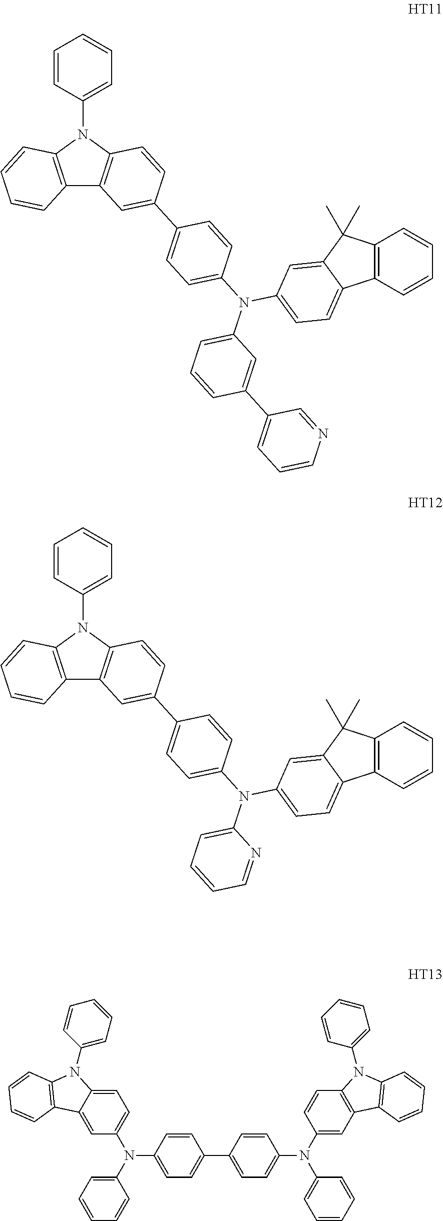 [SMILES: CC1(C)c2ccccc2-c2ccc(N(c3ccc(-c4ccc5c(c4)c4ccccc4n5-c4ccccc4)cc3)c3cccc(-c4cccnc4)c3)cc21 ; CC1(C)c2ccccc2-c2ccc(N(c3ccc(-c4ccc5c(c4)c4ccccc4n5-c4ccccc4)cc3)c3ccccn3)cc21 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1]

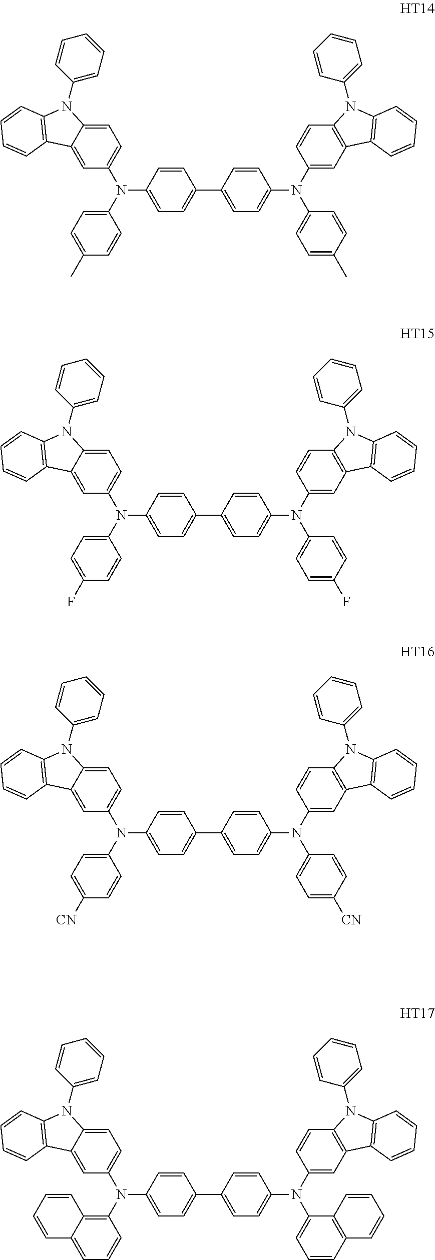

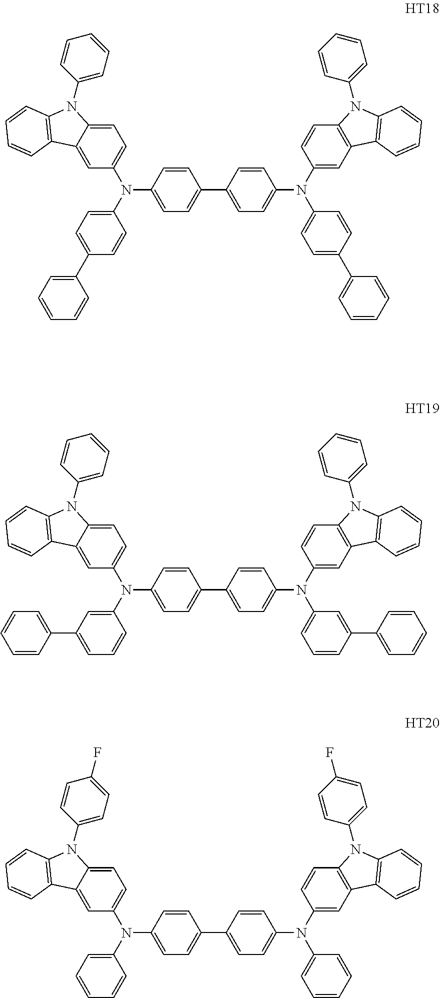 [SMILES: c1ccc(-c2ccc(N(c3ccc(-c4ccc(N(c5ccc(-c6ccccc6)cc5)c5ccc6c(c5)c5ccccc5n6-c5ccccc5)cc4)cc3)c3ccc4c(c3)c3ccccc3n4-c3ccccc3)cc2)cc1 ; c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1 ; Fc1ccc(-n2c3ccccc3c3cc(N(c4ccccc4)c4ccc(-c5ccc(N(c6ccccc6)c6ccc7c(c6)c6ccccc6n7-c6ccc(F)cc6)cc5)cc4)ccc32)cc1]

A thickness of the hole transport region may be in a range of about 100 Å to about 10,000 Å, for example, about 100 Å to about 1,000 Å. When the hole transport region includes both a hole injection layer and a hole transport layer, a thickness of the hole injection layer may be in a range of about 100 Å to about 10,000 Å, or, for example, about 100 Å to about 1,000 Å, and a thickness of the hole transport layer may be in a range of about 50 Å to about 2,000 Å, or, for example, about 100 Å to about 1,500 Å. When thicknesses of the hole transport region, the hole injection layer, and the hole transport layer are within these ranges, hole transporting properties of the hole transport region may be satisfactory without a substantial increase in a driving voltage.

The hole transport region may further include a charge-generating material to improve conductive properties in addition to the materials stated above. The charge-generating material may be homogeneously or non-homogeneously dispersed throughout the hole transport region.

The charge-generating material may be, for example, a p-dopant. The p-dopant may be one a quinone derivative, a metal oxide, or a cyano group-containing compound, as examples. For example, the p-dopant may be a quinone derivative, such as tetracyanoquinonedimethane (TCNQ) or 2,3,5,6-tetrafluoro-tetracyano-1,4-benzoquinonedimethane (F4-TCNQ); a metal oxide, such as a tungsten oxide or a molybdenum oxide, and Compound HT-D1:

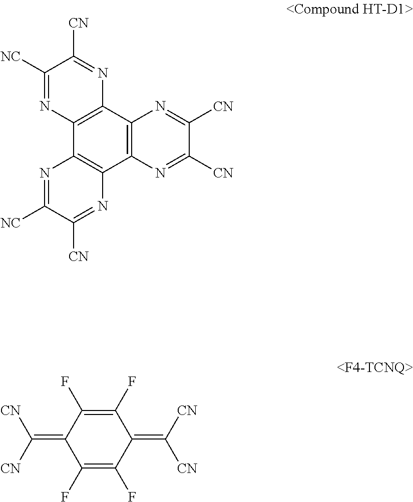

The hole transport region may further include at least one selected from a buffer layer and an electron blocking layer in addition to the hole injection layer and the hole transport layer. The buffer layer may compensate an optical resonance distance according to a wavelength of light emitted from the emission layer. Accordingly, light-emission efficiency of the organic light-emitting device including the buffer layer may improve. A material in the buffer layer may be a material that is also included in the hole transport region. The electron blocking layer may prevent injection of electrons from the electron transport region.

An emission layer may be formed on the first electrode 110 or the hole transport region by using various methods, such as vacuum deposition, spin coating, casting, a LB method, ink jet printing, laser-printing, or LITI. When the emission layer is formed by vacuum deposition or spin coating, deposition and coating conditions for forming the emission layer may be determined by referring to the deposition and coating conditions for forming the hole injection layer.

When the organic light-emitting device 10 is a full color organic light-emitting device, the emission layer may be patterned into sub-pixels including a red emission layer, a green emission layer, or a blue emission layer. In some implementations, the emission layer may have a stacked structure of a red emission layer, a green emission layer, and a blue emission layer, or may include a red-light emission material, a green-light emission material, and a blue-light emission material that are mixed with each other in a single layer, to emit white light.

The emission layer may include the condensed cyclic compound represented by Formula 1.

The emission layer may include a host and a dopant. The host may include the condensed cyclic compound represented by Formula 1.

The host may include at least one of TPBi, TBADN, ADN (also, referred to as “DNA”), CBP, CDBP, and TCP in addition to the condensed cyclic compound represented by Formula 1:

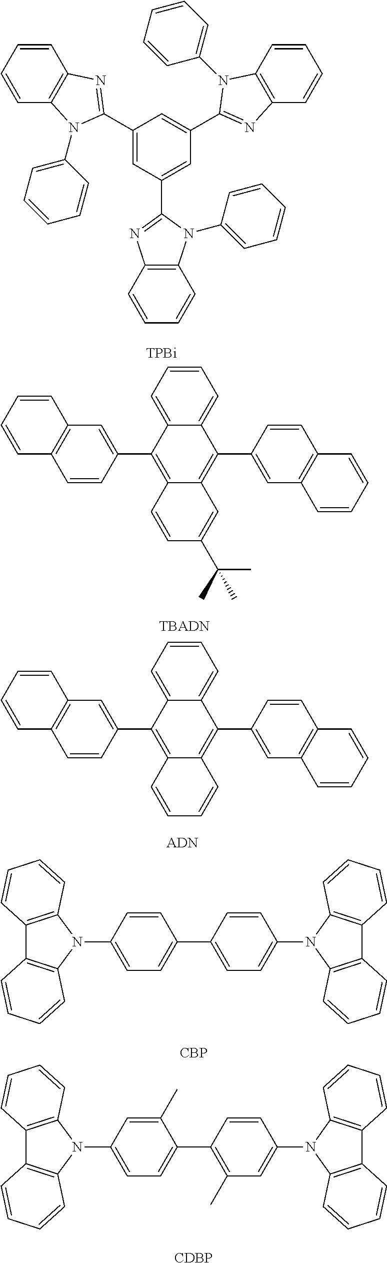

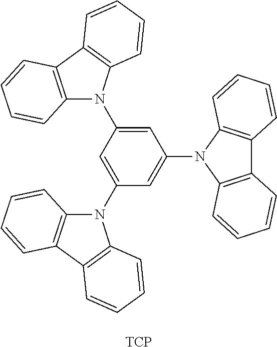

The dopant may include at least one of a fluorescent dopant and a phosphorescent dopant.

The phosphorescent dopant may include an organic metal complex represented by Formula 401:

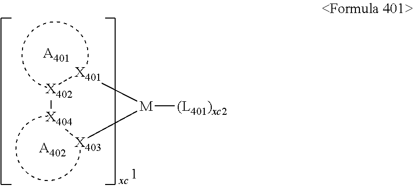

In Formula 401,

M may be selected from iridium (Ir), platinum (Pt), osmium (Os), titanium (Ti), zirconium (Zr), hafnium (Hf), europium (Eu), terbium (Tb), and thulium (Tm);

X₄₀₁ to X₄₀₄ may each independently be nitrogen or carbon;

rings A₄₀₁ and A₄₀₂ may each be independently selected from a substituted or unsubstituted benzene, a substituted or unsubstituted naphthalene, a substituted or unsubstituted fluorene, a substituted or unsubstituted spiro-fluorene, a substituted or unsubstituted indene, a substituted or unsubstituted pyrrole, a substituted or unsubstituted thiophene, a substituted or unsubstituted furan, a substituted or unsubstituted imidazole, a substituted or unsubstituted pyrazole, a substituted or unsubstituted thiazole, a substituted or unsubstituted isothiazole, a substituted or unsubstituted oxazole, a substituted or unsubstituted isooxazole, a substituted or unsubstituted pyridine, a substituted or unsubstituted pyrazine, a substituted or unsubstituted pyrimidine, a substituted or unsubstituted pyridazine, a substituted or unsubstituted quinoline, a substituted or unsubstituted isoquinoline, a substituted or unsubstituted benzoquinoline, a substituted or unsubstituted quinoxaline, a substituted or unsubstituted quinazoline, a substituted or unsubstituted carbazole, a substituted or unsubstituted benzoimidazole, a substituted or unsubstituted benzofuran, a substituted or unsubstituted benzothiophene, a substituted or unsubstituted isobenzothiophene, a substituted or unsubstituted benzoxazole, a substituted or unsubstituted isobenzoxazole, a substituted or unsubstituted triazole, a substituted or unsubstituted oxadiazole, a substituted or unsubstituted triazine, a substituted or unsubstituted dibenzofuran, and a substituted or unsubstituted dibenzothiophene;

at least one substituent of the substituted benzene, substituted naphthalene, substituted fluorene, substituted spiro-fluorene, substituted indene, substituted pyrrole, substituted thiophene, substituted furan, substituted imidazole, substituted pyrazole, substituted thiazole, substituted isothiazole, substituted oxazole, substituted isoxazole, substituted pyridine, substituted pyrazine, substituted pyrimidine, substituted pyridazine, substituted quinoline, substituted isoquinoline, substituted benzoquinoline, substituted quinoxaline, substituted quinazoline, substituted carbazole, substituted benzoimidazole, substituted benzofuran, substituted benzothiophene, substituted isobenzothiophene, substituted benzoxazole, substituted isobenzoxazole, substituted triazole, substituted oxadiazole, substituted triazine, substituted dibenzofuran, and substituted dibenzothiophene is selected from:

a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, and a C₁-C₆₀ alkoxy group;

a C₁-C₆₀ alkyl group, a C₂-C₆₀ alkenyl group, a C₂-C₆₀ alkynyl group, and a C₁-C₆₀ alkoxy group, each substituted with at least one selected from a deuterium, —F, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₄₀₁)(Q₄₀₂), —Si(Q₄₀₃)(Q₄₀₄)(Q₄₀₅), and —B(Q₄₀₆)(Q₄₀₇);

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group;

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₁-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₄₁₁)(Q₄₁₂), —Si(Q₄₁₃)(Q₄₁₄)(Q₄₁₅), and —B(Q₄₁₆)(Q₄₁₇); and

—N(Q₄₂₁)(Q₄₂₂), —Si(Q₄₂₃)(Q₄₂₄)(Q₄₂₅), and —B(Q₄₂₆)(Q₄₂₇),

L₄₀₁ may be an organic ligand;

xc1 may be 1, 2, or 3;

xc2 may be 0, 1, 2, or 3.

L₄₀₁ may be a monovalent, divalent, or trivalent organic ligand. For example, L₄₀₁ may be selected from a halogen ligand (for example, Cl or F), a diketone ligand (for example, acetylacetonate, 1,3-diphenyl-1,3-propandionate, 2,2,6,6-tetramethyl-3,5-heptanedionate, or hexafluoroacetonate), a carboxylic acid ligand (for example, picolinate, dimethyl-3-pyrazolecarboxylate, or benzoate), a carbon monoxide ligand, an isonitrile ligand, a cyano ligand, and a phosphorus ligand (for example, phosphine or phosphite).

In Formula 401, when A₄₀₁ has two or more substituents, the two or more substituents of A₄₀₁ may be linked to each other to form a saturated or unsaturated ring.

In Formula 401, when A₄₀₂ has two or more substituents, the two or more substituents of A₄₀₂ may be linked to each other to form a saturated or unsaturated ring.

In Formula 401, when xc1 is two or greater, a plurality of ligands

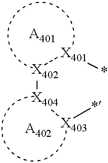

in Formula 401 may be identical to or different from each other. In Formula 401, when xc1 is two or greater, A₄₀₁ and A₄₀₂ may be, respectively, directly linked to A₄₀₁ and A₄₀₂ of a different neighboring ligand or may link to A₄₀₁ and A₄₀₂ of a different neighboring ligand via a linking group (e.g. a C₁-C₅ alkylene group, —N(R′)— (where, R′ is a C₁-C₁₀ alkyl group or a C₆-C₂₀ aryl group), or —C(═O)—) therebetween.

The fluorescent dopant may include at least one selected from DPAVBi, BDAVBi, TBPe, DCM, DCJTB, Coumarin 6, and C545T.

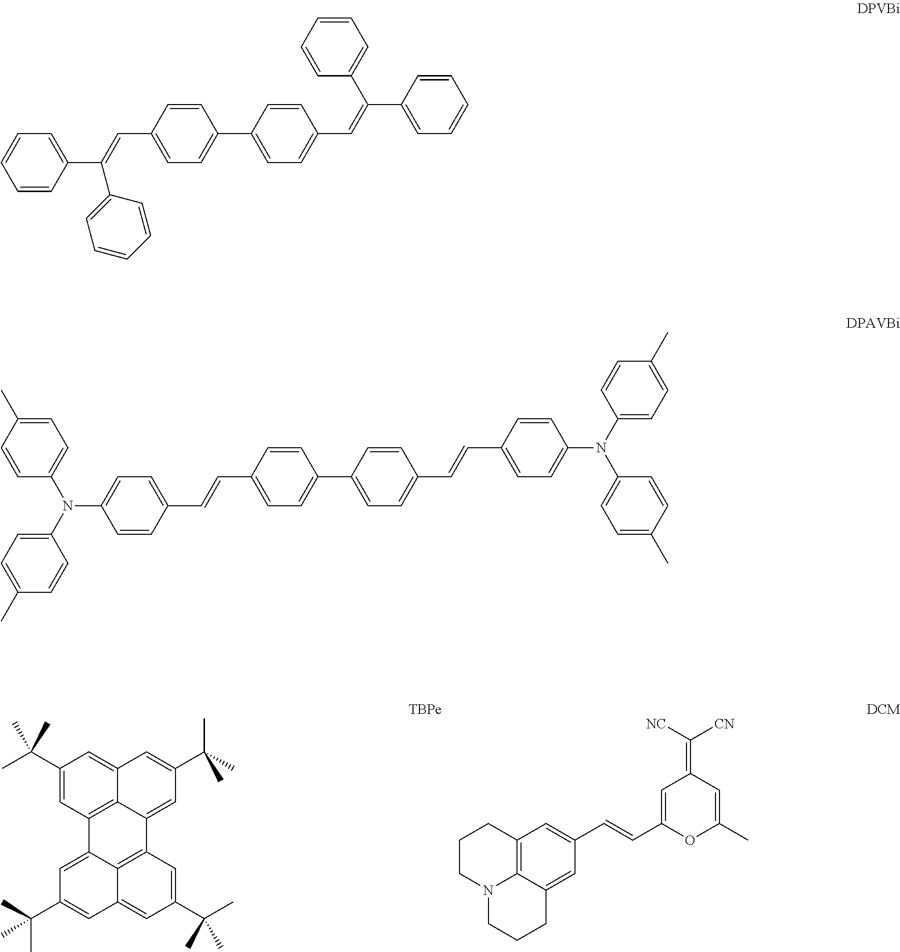

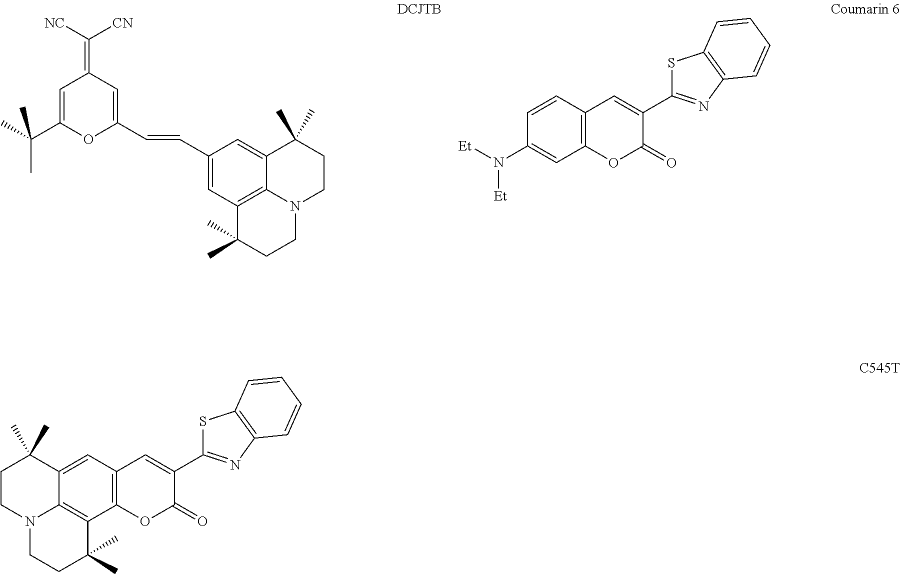

In some implementations, the fluorescent dopant may include a compound represented by Formula 501:

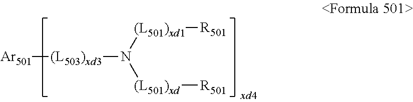

In Formula 501,

Ar₅₀₁ may be selected from:

a naphthalene, a heptalene, a fluorenene, a spiro-fluorene, a benzofluorene, a dibenzofluorene, a phenalene, a phenanthrene, an anthracene, a fluoranthene, a triphenylene, a pyrene, a chrysene, a naphthacene, a picene, a perylene, a pentaphene, and an indenoanthracene; and

a naphthalene, a heptalene, a fluorene, a spiro-fluorene, a benzofluorene, a dibenzofluorene, a phenalene, a phenanthrene, an anthracene, a fluoranthene, a triphenylene, a pyrene, a chrysene, naphthacene, a picene, a perylene, a pentaphene, and an indenoanthracene, each substituted with at least one selected from a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, and —Si(Q₅₀₁)(Q₅₀₂)(Q₅₀₃) (where, Q₅₀₁ to Q₅₀₃ are each independently selected from a hydrogen, a C₁-C₆₀ alkyl group, a C₂-C₆₀ alkenyl group, a C₆-C₆₀ aryl group, and a C₁-C₆₀ heteroaryl group);

L₅₀₁ to L₅₀₃ may have the same definition as L₂₀₁;

R₅₀₁ and R₅₀₂ may be each independently selected from:

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazole group, a triazinyl group, a dibenzofuranyl group, and a dibenzothiophenyl group; and

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, a triazinyl group and a dibenzofuranyl group, and a dibenzothiophenyl group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₁-C₂₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, a triazinyl group, a dibenzofuranyl group, and a dibenzothiophenyl group,

xd1 to xd3 may be each independently selected from 0, 1, 2, and 3; and

xb4 may be selected from 1, 2, 3, and 4.

An amount of the dopant in the emission layer may be typically in a range of about 0.01 part to about 15 parts by weight based on 100 parts by weight of the host.

A thickness of the emission layer may be in a range of about 100 Å to about 1,000 Å, or, for example, about 200 Å to about 600 Å. When a thickness of the emission layer is within this range, light-emission characteristics of the emission layer may be excellent without a substantial increase in a driving voltage.

An electron transport region may be disposed on the emission layer.

The electron transport region may include at least one selected from a hole blocking layer, an electron transport layer (ETL), and an electron injection layer.

For example, the electron transport region may have a structure of electron transport layer/electron injection layer or a structure of hole blocking layer/electron transport layer/electron injection layer, wherein layers of each structure are sequentially stacked on from the emission layer in the stated order.

In some embodiments, the organic layer 150 of the organic light-emitting device 10 may include an electron transport region disposed between the emission layer and the second electrode 190. The condensed cyclic compound represented by Formula 1 may be included in the electron transport region.

The electron transport region may include a hole blocking layer. When the emission layer includes a phosphorescent dopant, the hole blocking layer may prevent diffusion of excitons or holes into an electron transport layer.

When the electron transport region includes a hole blocking layer, the hole blocking layer may be formed on the emission layer by using a suitable method, such as vacuum deposition, spin coating, casting, a LB method, ink-jet printing, laser-printing, or LITI. When the hole blocking layer is formed by vacuum deposition or spin coating, deposition and coating conditions for forming the hole blocking layer may be determined by referring to the deposition and coating conditions for forming the hole injection layer.

The hole blocking layer may include, for example, at least one selected from BCP and Bphen.

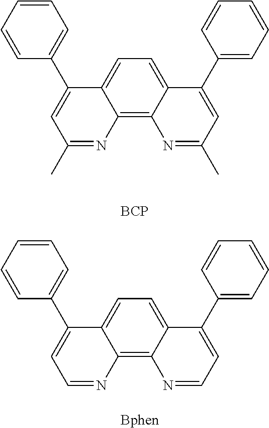

A thickness of the hole blocking layer may be in a range of about 20 Å to about 1,000 Å, or, for example, about 30 Å to about 300 Å. When a thickness of the hole blocking layer is within this range, hole blocking characteristics of the hole blocking layer may be excellent without a substantial increase in a driving voltage.

The electron transport region may include an electron transport layer. The electron transport layer may be formed on the emission layer or the hole blocking layer using a suitable method, such as vacuum deposition, spin coating, casting, a LB method, ink jet printing, laser-printing, or LITI. When an electron transport layer is formed by vacuum deposition or spin coating, vacuum deposition and coating conditions for forming the electron transport layer may be determined by referring to the vacuum deposition and coating conditions for forming the hole injection layer.

In some embodiments, the organic layer 150 of the organic light-emitting device 10 may include an electron transport region disposed between the emission layer and the second electrode 190. The electron transport region may include at least one selected from an electron transport layer and an electron injection layer.

The electron transport layer may include at least one selected from BCP, Bphen, Alq₃, BAlq, and NTAZ.

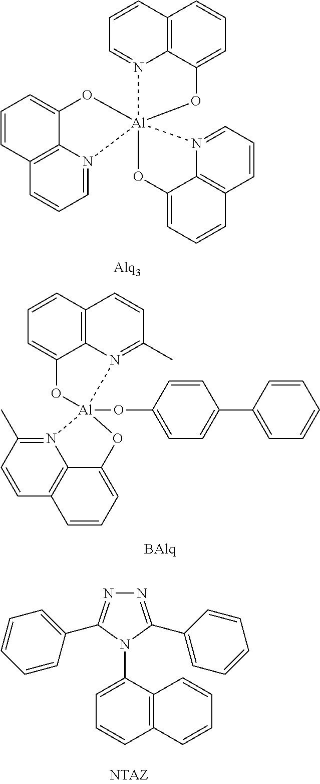

In some embodiments, the electron transport layer may include at least one compound selected from a compound represented by Formula 601 and a compound represented by Formula 602:

Ar₆₀₁-[(L₆₀₁)ₓₑ₁-E₆₀₁]ₓₑ₂ <Formula 601>

In Formula 601,

Ar₆₀₁ may be selected from:

a naphthalene, a heptalene, a fluorene, a spiro-fluorene, a benzofluorene, a dibenzofluorene, a phenalene, a phenanthrene, an anthracene, a fluoranthene, a triphenylene, a pyrene, a chrysene, a naphthacene, a picene, a perylene, a pentaphene, and an indenoanthracene;

a naphthalene, a heptalene, a fluorene, a spiro-fluorene, a benzofluorene, a dibenzofluorene, a phenalene, a phenanthrene, an anthracene, a fluoranthene, a triphenylene, a pyrene, a chrysene, a naphthacene, a picene, a perylene, a pentaphene, and an indenoanthracene, each substituted with at least one selected from a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, and —Si(Q₃₀₁)(Q₃₀₂)(Q₃₀₃) (where Q₃₀₁ to Q₃₀₃ are each independently selected from a hydrogen, a C₁-C₆₀ alkyl group, a C₂-C₆₀ alkenyl group, a C₆-C₆₀ aryl group, and a C₁-C₆₀ heteroaryl group);

L₆₀₁ may have the same definition as L₂₀₁;

E₆₀₁ may be selected from:

a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzoxazolyl group, an isobenzooxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, a thiadiazolyl group, an imidazopyridinyl group, and an imidazopyrimidinyl group; and

a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzoxazolyl group, an isobenzooxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, a thiadiazolyl group, an imidazopyridinyl group, and an imidazopyrimidinyl group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzoxazolyl group, an isobenzooxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, a thiadiazolyl group, an imidazopyridinyl group, and an imidazopyrimidinyl group;

xe1 may be selected from 0, 1, 2, and 3;

xe2 may be selected from 1, 2, 3, and 4;

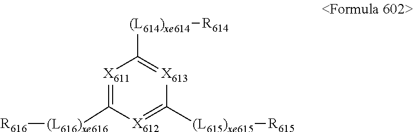

In Formula 602,

X₆₁₁ may be N or C-(L₆₁₁)ₓₑ₆₁₁-R₆₁₁, X₆₁₂ may be N or C-(L₆₁₂)ₓₑ₆₁₂-R₆₁₂, X₆₁₃ may be N or C-(L₆₁₃)ₓₑ₆₁₃-R₆₁₃, and at least one selected from X₆₁₁ to X₆₁₃ may be N;

L₆₁₁ to L₆₁₆ may each have the same definition as L₂₀₁;

R₆₁₁ to R₆₁₆ may be each independently selected from:

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group; and

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, C₁-C₂₀ alkyl group, C₁-C₂₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group; and

xe611 to xe616 may be each independently selected from 0, 1, 2, and 3.

The compound represented by Formula 601 and the compound represented by Formula 602 may be one of Compounds ET1 to ET15:

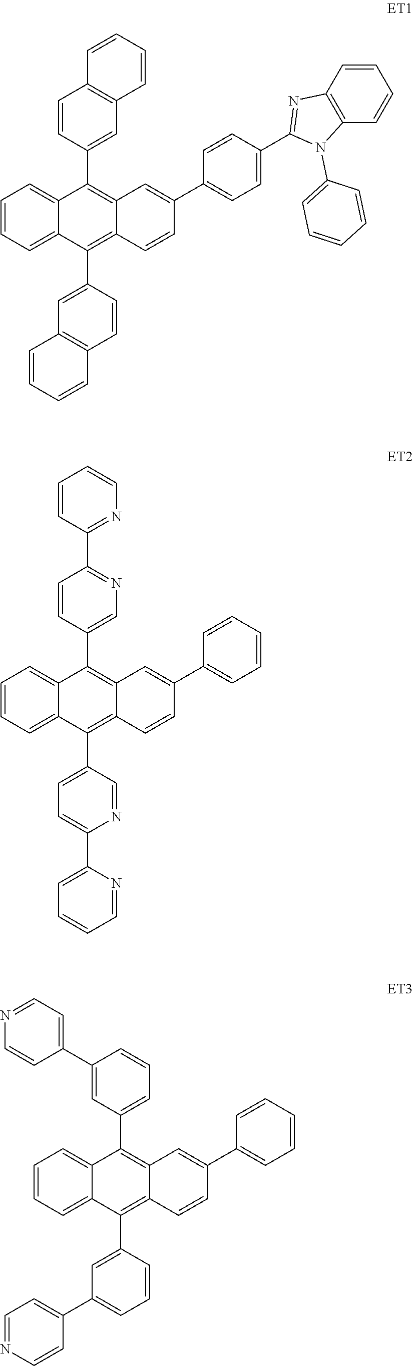 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

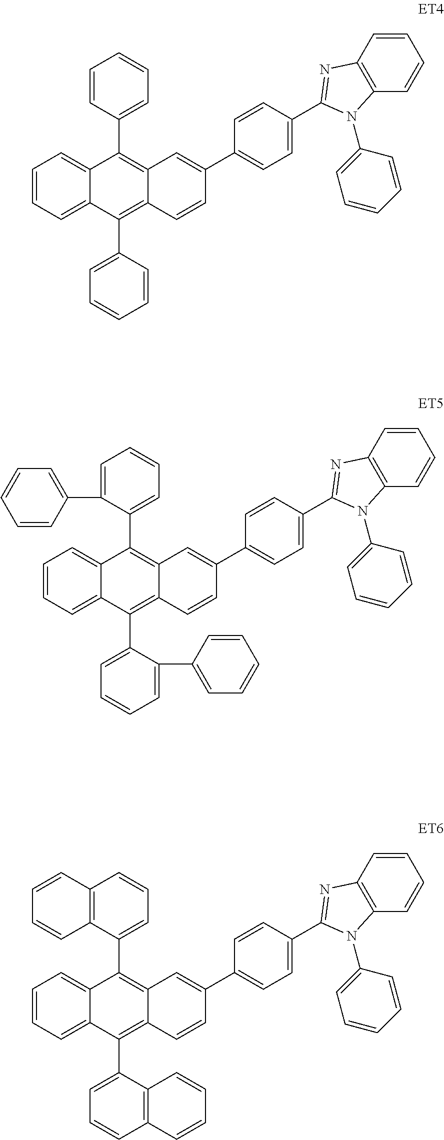 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

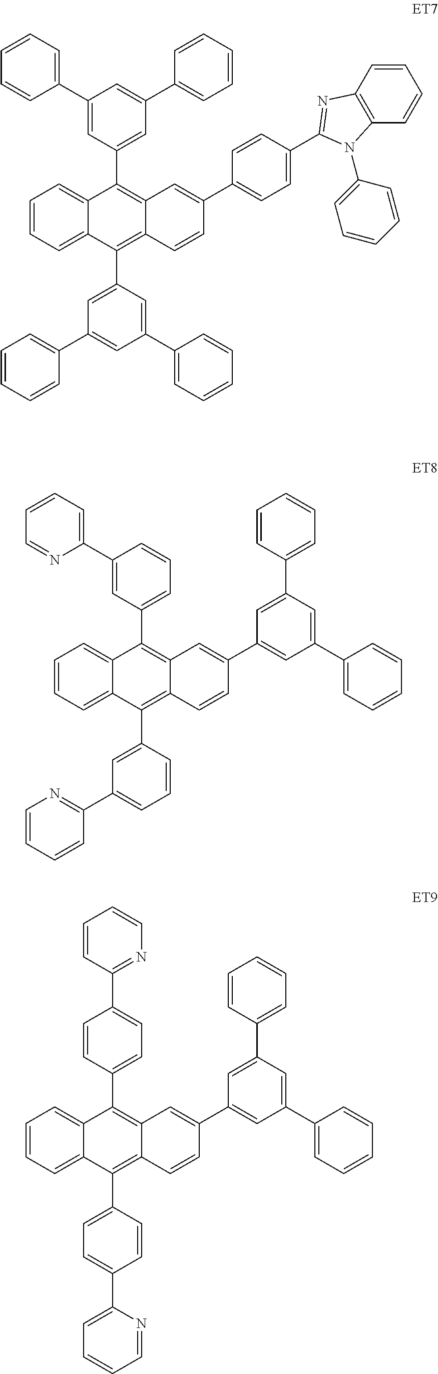 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1]

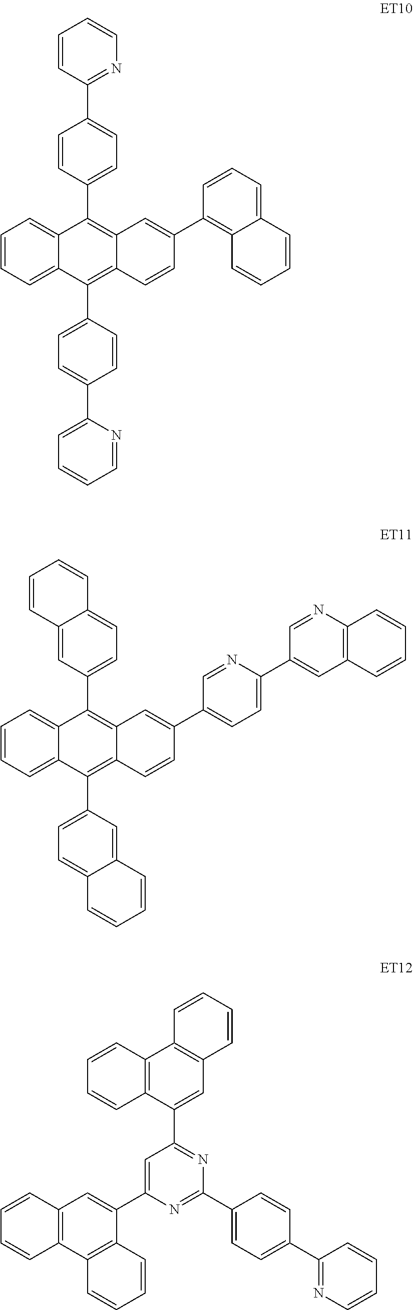 [SMILES: c1ccc(-c2ccc(-c3c4ccccc4c(-c4ccc(-c5ccccn5)cc4)c4cc(-c5cccc6ccccc56)ccc34)cc2)nc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc(-c2ccc(-c3nc(-c4cc5ccccc5c5ccccc45)cc(-c4cc5ccccc5c5ccccc45)n3)cc2)nc1]

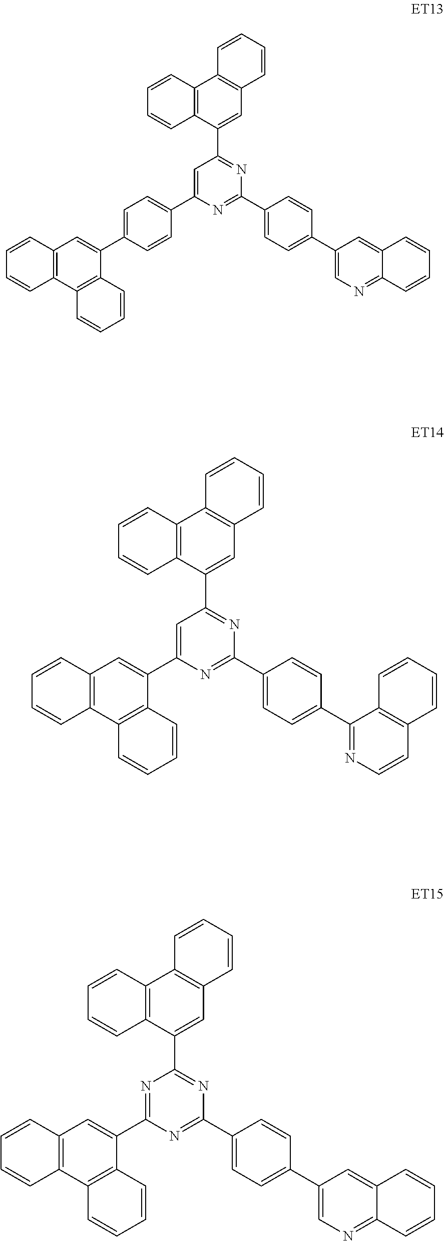 [SMILES: c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1 ; c1ccc2ncc(-c3ccc(-c4nc(-c5cc6ccccc6c6ccccc56)nc(-c5cc6ccccc6c6ccccc56)n4)cc3)cc2c1]

A thickness of the electron transport layer may be in a range of about 100 Å to about 1,000 Å, or, for example, about 150 Å to about 500 Å. When a thickness of the electron transport layer is within this range, hole transport characteristics of the electron transport layer may be excellent without a substantial increase in a driving voltage.

Also, the electron transport layer may further include a metal-containing material in addition to the materials described above.

The metal-containing material may include a Li complex. The Li complex may include, for example, Compound ET-D1 (lithium quinolate, LiQ) or ET-D2.

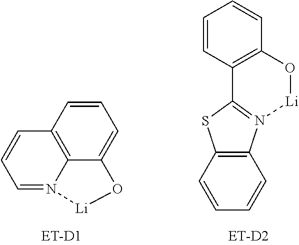

The electron transport region may include an electron injection layer that facilitates electron injection from the second electrode 190.

The electron injection layer may be formed on the electron transport layer by a suitable method, such as vacuum deposition, spin coating, casting, a LB method, ink-jet printing, laser-printing, or LITI. When an electron injection layer is formed by vacuum deposition or spin coating, vacuum deposition and coating conditions for the electron injection layer may be determined by referring to the vacuum deposition and coating conditions for the hole injection layer.

The electron injection layer may include at least one selected from, LiF, NaCl, CsF, Li₂O, BaO, and LiQ.

A thickness of the electron injection layer may be in a range of about 1 Å to about 100 Å, or, for example, about 3 Å to about 90 Å. When a thickness of the electron injection layer is within this range, electron injection characteristics of the electron injection layer may be satisfactory without a substantial increase in a driving voltage.

The second electrode 190 is disposed on the organic layer 150. The second electrode 190 may be a cathode that is an electron injection electrode. A second electrode material for forming the second electrode 190 may be a material having a low work function such a metal, an alloy, an electrically conductive compound, or a mixture thereof. Examples of the second electrode material may include lithium (Li), magnesium (Mg), aluminum (Al), aluminum-lithium (Al—Li), calcium (Ca), magnesium-indium (Mg—In), and magnesium-silver (Mg—Ag). In some embodiments, the second electrode material may be ITO or IZO. The second electrode 190 may be a reflective electrode, a semi-transmissive electrode, or a transmissive electrode.

Hereinbefore, the organic light-emitting device 10 has been described with reference to FIG. 1. In other implementations, other structures are possible.

The term “C₁-C₆₀ alkyl group” used herein refers to a linear or branched aliphatic monovalent hydrocarbon group having 1 to 60 carbon atoms. Examples of the C₁-C₆₀ alkyl group may include a methyl group, an ethyl group, a propyl group, an isobutyl group, a sec-butyl group, a tert-butyl group, a pentyl group, an iso-amyl group, and a hexyl group. The term “C₁-C₆₀ alkylene group” used herein refers to a divalent group having a same structure as a C₁-C₆₀ alkyl group.

The term “C₁-C₆₀ alkoxy group” used herein refers to a monovalent group represented by —OA₁₀₁ (where, A₁₀₁ is the C₁-C₆₀ alkyl group). Examples of the C₁-C₆₀ alkoxy group may include a methoxy group, an ethoxy group, and an isopropyloxy group.

The term “C₂-C₆₀ alkenyl group” used herein refers to a hydrocarbon group including at least one carbon double bond in the middle or at a terminal of the C₂-C₆₀ alkyl group. Examples of the C₂-C₆₀ alkenyl group may include an ethenyl group, a prophenyl group, and a butenyl group. The term “C₂-C₆₀ alkenylene group” used herein refers to a divalent group having a same structure as a C₂-C₆₀ alkenyl group.

The term “C₂-C₆₀ alkynyl group” used herein refers to a hydrocarbon group including at least one carbon triple bond in the middle or at a terminal of the C₂-C₆₀ alkyl group. Examples of the C₂-C₆₀ alkynyl group may include an ethynyl group and a propynyl group. The term “C₂-C₆₀ alkynylene group” used herein refers to a divalent group having a same structure as a C₂-C₆₀ alkynyl group.

The term “C₃-C₁₀ cycloalkyl group” used herein refers to a monovalent monocyclic saturated hydrocarbon group including 3 to 10 carbon atoms. Examples of the C₃-C₁₀ cycloalkyl group may include a cyclopropyl group, a cyclobutyl group, a cyclopentyl group, a cyclohexyl group, and a cycloheptyl group. the term “C₃-C₁₀ cycloalkylene group” used herein refers to a divalent group having a same structure as a C₃-C₁₀ cycloalkyl group.

The term “C₁-C₁₀ heterocycloalkyl group” used herein refers to a monovalent monocyclic group including at least one hetero atom selected from N, O, P, and S as a ring-forming atom and 1 to 10 carbon atoms. Examples of the C₁-C₁₀ heterocycloalkyl group may include a tetrahydrofuranyl group and a tetrahydrothiophenyl group. The term “C₁-C₁₀ heterocycloalkylene group” used herein refers to a divalent group having a same structure as a C₁-C₁₀ heterocycloalkyl group.

The term “C₃-C₁₀ cycloalkenyl group” used herein refers to a monovalent monocyclic group including 3 to 10 carbon atoms and at least one double bond in the ring of the C₃-C₁₀ cycloalkenyl group, and does not have aromacity. Examples of the C₃-C₁₀ cycloalkenyl group may include a cyclopentenyl group, a cyclohexenyl group, and a cycloheptenyl group. The term “C₃-C₁₀ cycloalkenylene group” used herein refers to a divalent group having the a structure as a C₃-C₁₀ cycloalkenyl group.

The term “C₁-C₁₀ heterocycloalkenyl group” used herein refers to a monovalent monocyclic group including at least one hetero atom selected from N, O, P, and S as a ring-forming atom, 1 to 10 carbon atoms, and at least one double bond in its ring. Examples of the C₁-C₁₀ heterocycloalkenyl group may include a 2,3-hydrofuranyl group and a 2,3-hydrothiophenyl group. The term “C₁-C₁₀ heterocycloalkenylene group” used herein refers to a divalent group having a same structure as a C₁-C₁₀ heterocycloalkenyl group.

The term “C₆-C₆₀ aryl group” used herein refers to a monovalent group including a carbocyclic aromatic system having 6 to 60 carbon atoms, and a C₆-C₆₀ arylene group used herein refers to a divalent group including a carbocyclic aromatic system having 6 to 60 carbon atoms. Examples of the C₆-C₆₀ aryl group may include a phenyl group, a naphthyl group, an anthracenyl group, a phenanthrenyl group, a pyrenyl group, and a chrysenyl group. When the C₆-C₆₀ aryl group and the C₆-C₆₀ arylene group each include two or more rings, the rings may be fused to each other.

The term “C₁-C₆₀ heteroaryl group” used herein refers to a monovalent group having a carbocyclic aromatic system including at least one hetero atom selected from N, O, P, and S as a ring-forming atom and 1 to 60 carbon atoms. The term “C₁-C₆₀ heteroarylene group” used herein refers to a divalent group having a carbocyclic aromatic system including at least one hetero atom selected from N, O, P, and S as a ring-forming atom and 1 to 60 carbon atoms. Examples of the C₁-C₆₀ heteroaryl group may include a pyridinyl group, a pyrimidinyl group, a pyrazinyl group, a pyridazinyl group, a triazinyl group, a quinolinyl group, and an isoquinolinyl group. When the C₁-C₆₀ heteroaryl group or the C₁-C₆₀ heteroarylene group include two or more rings, the rings may be fused to each other.

The term “C₆-C₆₀ aryloxy group” used herein denotes —OA₁₀₂ (where, A₁₀₂ is the C₆-C₆₀ aryl group), and the term “C₆-C₆₀ arylthio group” used herein denotes —SA₁₀₃ (where, A₁₀₃ is the C₆-C₆₀ aryl group).

The term “monovalent non-aromatic condensed polycyclic group” used herein refers to a monovalent group (for example, having 8 to 60 carbon atoms) that has two or more rings condensed to each other, only carbon atoms as a ring forming atom, and non-aromacity in the entire molecular structure. Examples of the monovalent non-aromatic condensed polycyclic group may include a fluorenyl group. A divalent non-aromatic condensed polycyclic group used herein refers to a divalent group having the same structure with the monovalent non-aromatic condensed polycyclic group.

The term “monovalent non-aromatic condensed heteropolycyclic group” used herein refers to a monovalent group (for example, having 1 to 60 carbon atoms) that has two or more rings condensed to each other, has a hetero atom selected from N, O, P, and S, other than carbon atoms, as a ring forming atom, and has non-aromacity in the entire molecular structure. Example of the monovalent non-aromatic condensed heteropolycyclic group may include a carbazolyl group. The term “divalent non-aromatic condensed hetero-polycyclic group” used herein refers to a divalent group having a same structure as a monovalent non-aromatic condensed hetero-polycyclic group.

As used herein, the at least one substituent of the substituted C₃-C₁₀ cycloalkylene group, substituted C₁-C₁₀ heterocycloalkylene group, substituted C₃-C₁₀ cycloalkenylene group, substituted C₁-C₁₀ heterocycloalkenylene group, substituted C₆-C₆₀ arylene group, substituted C₁-C₆₀ heteroarylene group, substituted divalent non-aromatic condensed polycyclic group, substituted divalent non-aromatic condensed heteropolycyclic group, substituted C₁-C₆₀ alkyl group, substituted C₂-C₆₀ alkenyl group, substituted C₂-C₆₀ alkynyl group, substituted C₁-C₆₀ alkoxy group, substituted C₃-C₁₀ cycloalkyl group, substituted C₁-C₁₀ heterocycloalkyl group, substituted C₃-C₁₀ cycloalkenyl group, substituted C₁-C₁₀ heterocycloalkenyl group, substituted C₆-C₆₀ aryl group, substituted C₆-C₆₀ aryloxy group, substituted C₆-C₆₀ arylthio group, substituted C₁-C₆₀ heteroaryl group, substituted monovalent non-aromatic condensed polycyclic group, and substituted monovalent non-aromatic condensed heteropolycyclic group is selected from:

a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, and a C₁-C₆₀ alkoxy group;

a C₁-C₆₀ alkyl group, a C₂-C₆₀ alkenyl group, a C₂-C₆₀ alkynyl group, and a C₆₀ alkoxy group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —Si(Q₁₁)(Q₁₂)(Q₁₃), —B(Q₁₄)(Q₁₅), and —N(Q₁₆)(Q₁₇);

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group;

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —Si(Q₂₁)(Q₂₂)(Q₂₃), —B(Q₂₄)(Q₂₅), and —N(Q₂₆)(Q₂₇); and

—Si(Q₃₁)(Q₃₂)(Q₃₃), —B(Q₃₄)(Q₃₅), and —N(Q₂₆)(Q₂₇),

wherein, Q₁₁ to Q₁₇, Q₂₁ to Q₂₇, and Q₃₁ to Q₃₇ may be each independently selected from a hydrogen, a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group.

In some embodiments, at least one substituent of the substituted C₃-C₁₀ cycloalkylene group, substituted C₁-C₁₀ heterocycloalkylene group, substituted C₃-C₁₀ cycloalkenylene group, substituted C₁-C₁₀ heterocycloalkenylene group, substituted C₆-C₆₀ arylene group, substituted C₁-C₆₀ heteroarylene group, substituted a divalent non-aromatic condensed polycyclic group, substituted a divalent non-aromatic condensed heteropolycyclic group, substituted C₁-C₆₀ alkyl group, substituted C₂-C₆₀ alkenyl group, substituted C₂-C₆₀ alkynyl group, substituted C₁-C₆₀ alkoxy group, substituted C₃-C₁₀ cycloalkyl group, substituted C₁-C₁₀ heterocycloalkyl group, substituted C₃-C₁₀ cycloalkenyl group, substituted C₁-C₁₀ heterocycloalkenyl group, substituted C₆-C₆₀ aryl group, substituted C₆-C₆₀ aryloxy group, substituted C₆-C₆₀ arylthio group, substituted C₁-C₆₀ heteroaryl group, substituted monovalent non-aromatic condensed polycyclic group, and substituted monovalent non-aromatic condensed heteropolycyclic group is selected from:

a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, and a C₁-C₆₀ alkoxy group;

a C₁-C₆₀ alkyl group, a C₂-C₆₀ alkenyl group, a C₂-C₆₀ alkynyl group, and a C₁-C₆₀ alkoxy group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a cyclopentyl group, a cyclohexyl group, a cycloheptyl group, a cyclopentenyl group, a cyclohexenyl group, a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzoxazolyl group, an isobenzooxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, a thiadiazolyl group, an imidazopyridinyl group, an imidazopyrimidinyl group, —Si(Q₁₁)(Q₁₂) (Q₁₃), —B(Q₁₄)(Q₁₅), and —N(Q₁₆)(Q₁₇);

a cyclopentyl group, a cyclohexyl group, a cycloheptyl group, a cyclopentenyl group, a cyclohexenyl group, a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzoxazolyl group, an isobenzooxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, a thiadiazolyl group, an imidazopyridinyl group, and an imidazopyrimidinyl group;

a cyclopentyl group, a cyclohexyl group, a cycloheptyl group, a cyclopentenyl group, a cyclohexenyl group, a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzoxazolyl group, an isobenzooxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, a thiadiazolyl group, an imidazopyridinyl group, and an imidazopyrimidinyl group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, a C₁-C₆₀ alkoxy group, a cyclopentyl group, a cyclohexyl group, a cycloheptyl group, a cyclopentenyl group, a cyclohexenyl group, a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzoxazolyl group, an isobenzooxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, a thiadiazolyl group, an imidazopyridinyl group, an imidazopyrimidinyl group, —Si(Q₂₁)(Q₂₂)(Q₂₃), —B(Q₂₄)(Q₂₅), and —N(Q₂₆)(Q₂₇); and

—Si(Q₃₁)(Q₃₂)(Q₃₃), —B(Q₃₄)(Q₃₅), and —N(Q₃₆)(Q₃₇);

wherein, Q₁₁ to Q₁₇, Q₂₁ to Q₂₇, and Q₃₁ to Q₃₇ are each independently selected from a hydrogen, a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, a C₁-C₆₀ alkoxy group, a cyclopentyl group, a cyclohexyl group, a cycloheptyl group, a cyclopentenyl group, a cyclohexenyl group, a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzoxazolyl group, an isobenzooxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, a thiadiazolyl group, an imidazopyridinyl group, and an imidazopyrimidinyl group.

As used herein, the expression “Ph” denotes a phenyl group, the expression “Me” denotes a methyl group, the expression “Et” denotes an ethyl group, and the expression “ter-Bu” or “Bu^{t}” denotes a tert-butyl group.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples. The wording “B was used instead of A” used in describing Synthesis Examples denotes that a molar equivalent of A was identical to a molar equivalent of B.

### Synthesis Example 1

### Synthesis of Compound 3

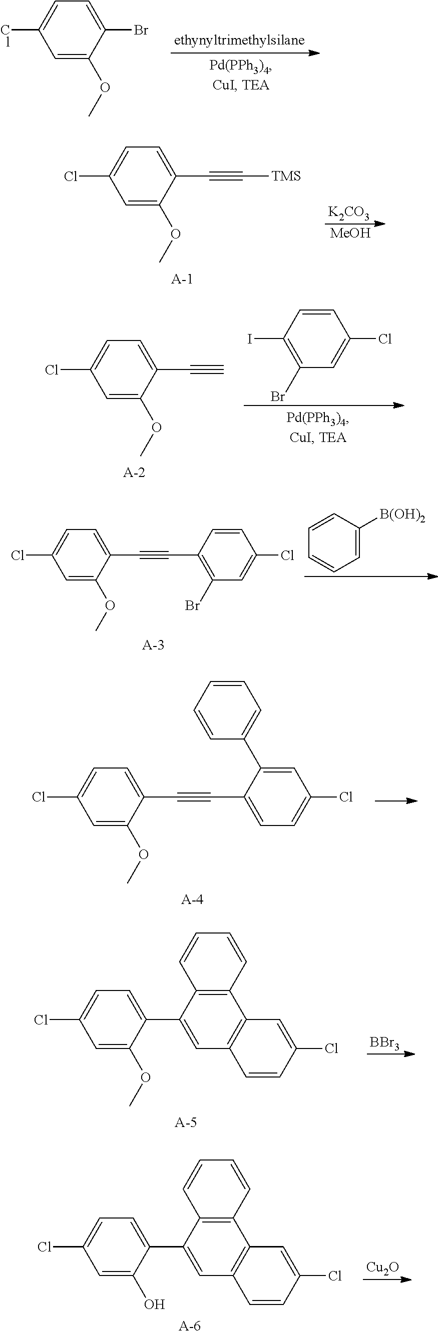

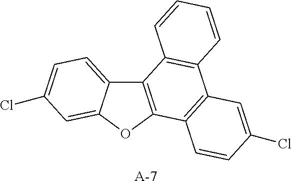

### Synthesis of Intermediate A-1

2.21 g (10 mmol) of 2-bromo-5-chloroanisole, 1.08 g (11 mmol) of ethynyltrimethylsilane, 0.58 g (0.5 mmol) of Pd(PPh₃)₄, 0.095 g (0.5 mmol) of CuI, and 4.18 ml (30 mmol) of TEA were dissolved in 200 ml of THF, and reflux-stirred at 80° C. for 4 hours. Then, an organic layer was extracted therefrom three times by using 40 ml of H₂O and 40 ml of ethylether The organic layer was dried using magnesium sulfate, and a solvent was removed therefrom by evaporation. The residue was separated and purified through a silica gel chromatography to obtain 1.91 g of Intermediate A-1 (yield: 80%). The compound thus obtained was confirmed by using LC-MS. (C₁₂H₁₅ClOSi; M+ calcd: 238.79. found: 238.87).

### Synthesis of Intermediate A-2

2.38 g (10 mmol) of Intermediate A-1 and 4.15 g (30 mmol) of potassium carbonate were dissolved in 100 ml of methanol and stirred at room temperature for 3 hours. Then, the reaction solution was distilled under a reduced pressure, an organic layer was extracted therefrom three times by using 40 ml of H₂O and 40 ml of ethylether. The organic layer was dried using magnesium sulfate, and a solvent was removed therefrom by evaporation. The residue was separated and purified through a silica gel chromatography to obtain 1.58 g of Intermediate A-2 (yield: 95%). The compound thus obtained was confirmed by using LC-MS. (C₉H₇ClO; M+ calcd: 166.60. found: 166.71).

### Synthesis of Intermediate A-3

1.67 g (10 mmol) of Intermediate A-2, 3.49 g (11 mmol) of 2-bromo-4-chloro-1-iodobenzene, 0.58 g (0.5 mmol) of Pd(PPh₃)₄, 0.095 g (0.5 mmol) of CuI, and 4.18 ml (30 mmol) of TEA were dissolved in 200 ml of THF, and reflux-stirred at 80° C. for 4 hours. Then, an organic layer was extracted therefrom three times by using 40 ml of H₂O and 40 ml of ethylether. The organic layer was dried using magnesium sulfate, and a solvent was removed therefrom by evaporation. The residue was separated and purified through a silica gel chromatography to obtain 2.85 g of Intermediate A-3 (yield: 80%). The compound thus obtained was confirmed by using LC-MS. (C₁₅H₉BrCl₂O; M+ calcd: 356.04. found: 356.20).

### Synthesis of Intermediate A-4

3.56 g (10 mmol) of Intermediate A-3, 1.22 g (11 mmol) of phenylboronic acid, 0.58 g (0.5 mmol) of Pd(PPh₃)₄, and 4.15 g (30 mmol) of potassium carbonate were dissolved in 200 ml of a THF:H₂O=2:1 (vol) solution, and reflux-stirred at 80° C. for 4 hours. Then, an organic layer was extracted therefrom three times by using 40 ml of H₂O and 40 ml of ethylether. The organic layer was dried using magnesium sulfate, and a solvent was removed therefrom by evaporation. The residue was separated and purified through a silica gel chromatography to obtain 3.00 g of Intermediate A-4 (yield: 85%). The compound thus obtained was confirmed by using LC-MS. (C₂₁H₁₄Cl₂O; M+ calcd: 353.24. found: 353.74).

### Synthesis of Intermediate A-5

3.53 g (10 mmol) of Intermediate A-4, 0.65 ml (10 mmol) of methanesulfonic acid, and 7.66 ml (100 mmol) of trifluoroacetic acid were dissolved in 100 ml of dichloromethane and stirred at 0° C. for 2 hours. Then, an organic layer was extracted therefrom three times with 50 ml of H₂O and 40 ml of dichloromethane. The organic layer was dried using magnesium sulfate, and a solvent was removed therefrom by evaporation. The residue was separated and purified through a silica gel chromatography to obtain 2.83 g of Intermediate A-5 (yield: 80%). The compound thus obtained was confirmed by using LC-MS. (C₂₁H₁₄Cl₂O; M+ calcd: 353.24. found: 353.57).

### Synthesis of Intermediate A-6

3.53 g (10 mmol) of Intermediate A-5 was dissolved in 100 ml of dichloromethane, and, at 0° C., in a nitrogen atmosphere, 12.5 ml (50 mmol) of borontribromide (1.0 M in DCM) was slowly and dropwisely added thereto. Then, an organic layer was extracted therefrom three times by using 50 ml of H₂O, stirring, and 40 ml of ethylether. The organic layer was dried using magnesium sulfate and a solvent was removed therefrom by evaporation. Next, the residue was separated and purified through a silica gel chromatography to obtain 2.71 g of Intermediate A-6 (yield: 80%). The compound thus obtained was confirmed by using LC-MS. (C₂₀H₁₂Cl₂O; M+ calcd: 339.22. found: 339.32).

### Synthesis of Intermediate A-7

3.39 g (10 mmol) of Intermediate A-6 and 7.15 g (50 mmol) of Cu₂O were dissolved in 80 ml of nitrobenzene and reflux-stirred at 180° C. for 12 hours. Then, an organic layer was extracted therefrom three times by using 50 ml of H₂O, stirring, and 40 ml of ethylether. The organic layer was dried using magnesium sulfate and a solvent was removed therefrom by evaporation. Next, the residue was separated and purified through a silica gel chromatography to obtain 2.69 g of Intermediate A-7 (yield: 80%). The compound thus obtained was confirmed by using LC-MS. (C₂₀H₁₀Cl₂O; M+ calcd: 337.20. found: 337.32).

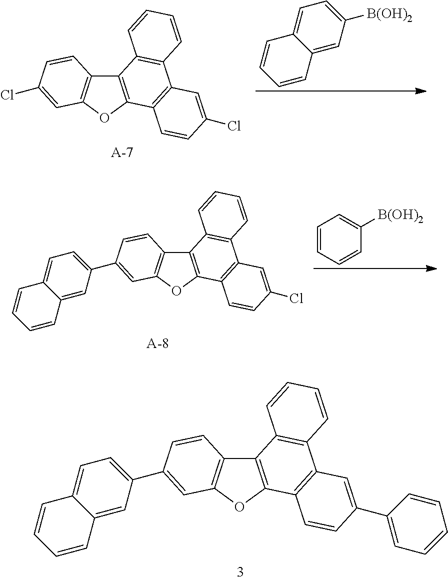

### Synthesis of Intermediate A-8

3.37 g (10 mmol) of Intermediate A-7, 1.89 g (11 mmol) of 2-naphthylboronic acid, 0.58 g (0.5 mmol) of Pd(PPh₃)₄, and 4.15 g (30 mmol) of potassium carbonate were dissolved in 200 ml of a THF:H₂O=2:1 (vol) solution, and reflux-stirred at 80° C. for 2 hours. Then, an organic layer was extracted therefrom three times by using 40 ml of H₂O and 40 ml of ethylether. The organic layer was dried using magnesium sulfate and a solvent was removed therefrom by evaporation. The residue was separated and purified through a silica gel chromatography to obtain 3.65 g of Intermediate A-8 (yield: 85%). The compound thus obtained was confirmed by using LC-MS. (C₃₀H₁₇ClO; M+ calcd: 428.92. found: 428.85).

### Synthesis of Compound 3

4.29 g (10 mmol) of Intermediate A-8, 1.33 g (11 mmol) of phenylboronic acid, 0.58 g (0.5 mmol) of Pd(PPh₃)₄, and 4.15 g (30 mmol) of potassium carbonate were dissolved in 200 ml of a THF:H₂O=2:1 (vol) solution, and reflux-stirred at 80° C. for 2 hours. Then, an organic layer was extracted therefrom three times by using 40 ml of H₂O and 40 ml of ethylether. The organic layer was dried using magnesium sulfate, and a solvent was removed therefrom by evaporation. The residue was separated and purified through a silica gel chromatography to obtain 3.65 g of Compound 3 (yield: 85%). The compound thus obtained was confirmed by using LC-MS and ¹H NMR. (C₃₆H₂₂O; M+ calcd: 470.57. found: 470.41).

### Synthesis Example 2

### Synthesis of Compound 9

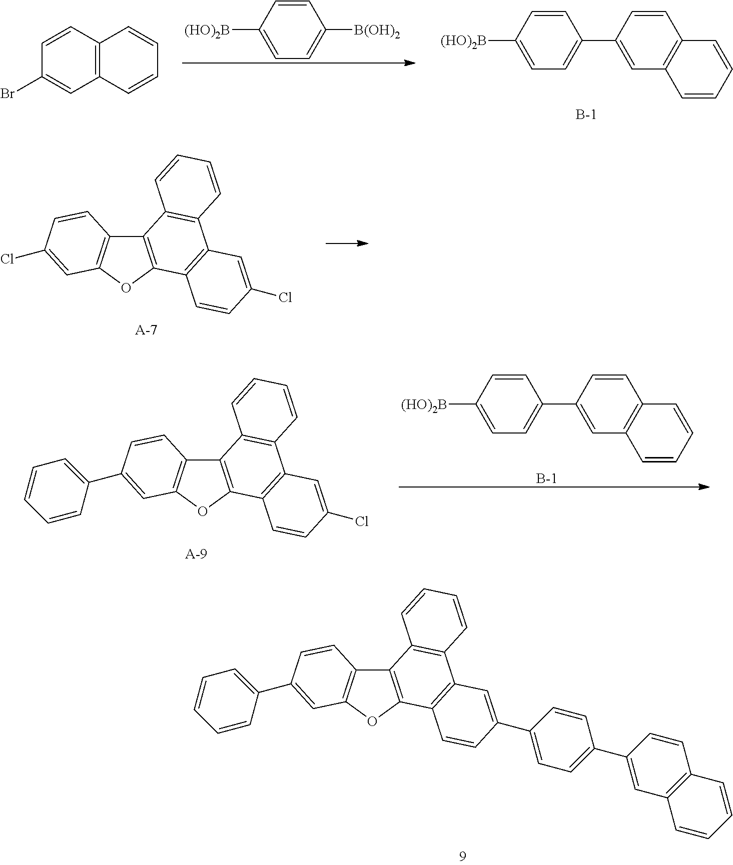

### Synthesis of Intermediate B-1

2.07 g (10 mmol) of 2-bromonaphthalene, 2.49 g (15 mmol) of benzene-1,4-diboronic acid, 0.58 g (0.5 mmol) of Pd(PPh₃)₄, and 4.15 g (30 mmol) of potassium carbonate were dissolved in 200 ml of a THF:H₂O=2:1 (vol) solution, and reflux-stirred at 80° C. for 2 hours. Then, an organic layer was extracted therefrom three times by using 40 ml of H₂O and 40 ml of ethylether. The organic layer was dried using magnesium sulfate, and a solvent was removed therefrom by evaporation. The residue was separated and purified through a silica gel chromatography to obtain 1.98 g of Intermediate B-1 (yield: 80%). The compound thus obtained was confirmed by using LC-MS. (C₁₆H₁₃BO₂; M+ calcd: 248.09. found: 248.24).

### Synthesis of Intermediate A-9

3.37 g (10 mmol) of Intermediate A-7, 1.33 g (11 mmol) of phenylboronic acid, 0.58 g (0.5 mmol) of Pd(PPh₃)₄, and 4.15 g (30 mmol) of potassium carbonate were dissolved in 200 ml of a THF:H₂O=2:1 (vol) solution, and reflux-stirred at 80° C. for 2 hours. Then, an organic layer was extracted therefrom three times by using 40 ml of H₂O and 40 ml of ethylether. The organic layer was dried using magnesium sulfate, and a solvent was removed therefrom by evaporation. The residue was separated and purified through a silica gel chromatography to obtain 3.22 g of Intermediate A-9 (yield: 85%). The compound thus obtained was confirmed by using LC-MS. (C₂₆H₁₅ClO; M+ calcd: 378.86. found: 378.58).

### Synthesis of Compound 9

3.79 g (10 mmol) of Intermediate A-9, 2.73 g (11 mmol) of Intermediate B-1, 0.58 g (0.5 mmol) of Pd(PPh₃)₄, and 4.15 g (30 mmol) of potassium carbonate were dissolved in 200 ml of a THF:H₂O=2:1 (vol) solution, and reflux-stirred at 80° C. for 4 hours. Then, an organic layer was extracted therefrom three times by using 40 ml of H₂O and 40 ml of ethylether. The organic layer was dried using magnesium sulfate, and a solvent was removed therefrom by evaporation. The residue was separated and purified through a silica gel chromatography to obtain 4.64 g of Compound 9 (yield: 85%). The compound thus obtained was confirmed by using LC-MS. (C₄₂H₂₆O; M+ calcd: 546.67. found: 546.87).

### Synthesis Example 3

### Synthesis of Compound 19

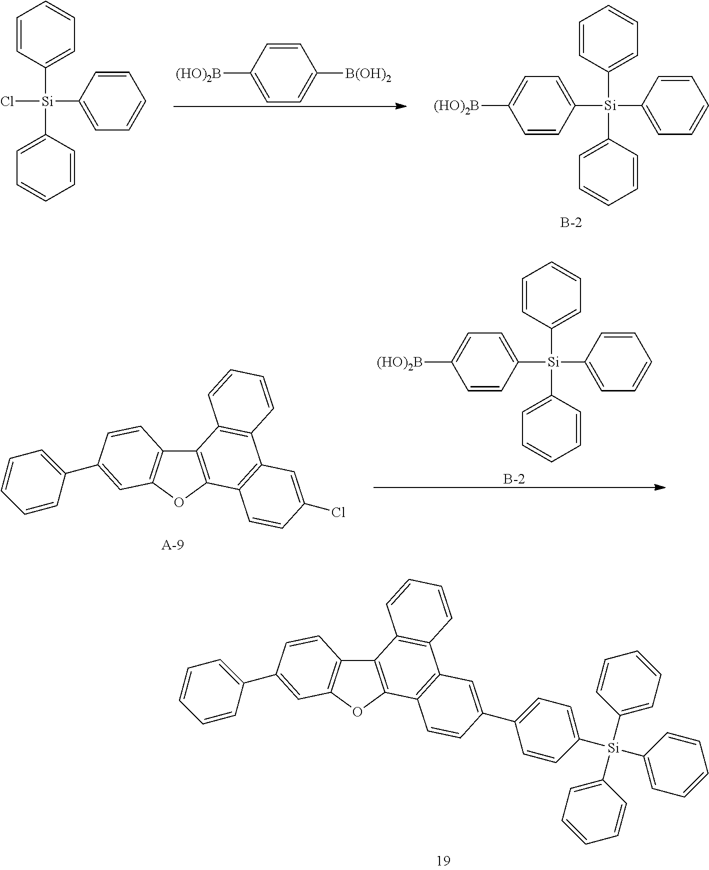

### Synthesis of Intermediate B-2

2.94 g (10 mmol) of chlorotriphenylsilane, 2.49 g (15 mmol) of benzene-1,4-diboronic acid, 0.58 g (0.5 mmol) of Pd(PPh₃)₄, and 4.15 g (30 mmol) of potassium carbonate were dissolved in 200 ml of a THF:H₂O=2:1 (vol) solution, and reflux-stirred at 80° C. for 2 hours. Then, an organic layer was extracted therefrom three times by using 40 ml of H₂O and 40 ml of ethylether. The organic layer was dried using magnesium sulfate, and a solvent was removed therefrom by evaporation. The residue was separated and purified through a silica gel chromatography to obtain 3.04 g of Intermediate B-2 (yield: 80%). The compound thus obtained was confirmed by using LC-MS. (C₂₄H₂₁BO₂Si; M+ calcd: 380.14. found: 380.54).

### Synthesis of Compound 19

3.79 g (10 mmol) of Intermediate A-9, 4.18 g (11 mmol) of Intermediate B-2, 0.58 g (0.5 mmol) of Pd(PPh₃)₄, and 4.15 g (30 mmol) of potassium carbonate were dissolved in 200 ml of a THF:H₂O=2:1 (vol) solution, and reflux-stirred at 80° C. for 4 hours. Then, an organic layer was extracted therefrom three times by using 40 ml of H₂O and 40 ml of ethylether. The organic layer was dried using magnesium sulfate, and a solvent was removed therefrom by evaporation. The residue was separated and purified through a silica gel chromatography to obtain 5.76 g of Compound 19 (yield: 85%). The compound thus obtained was confirmed by using LC-MS and ¹H NMR. (C₅₀H₃₄OSi; M+ calcd: 678.24. found: 678.21).

### Synthesis Example 4

### Synthesis of Compound 27

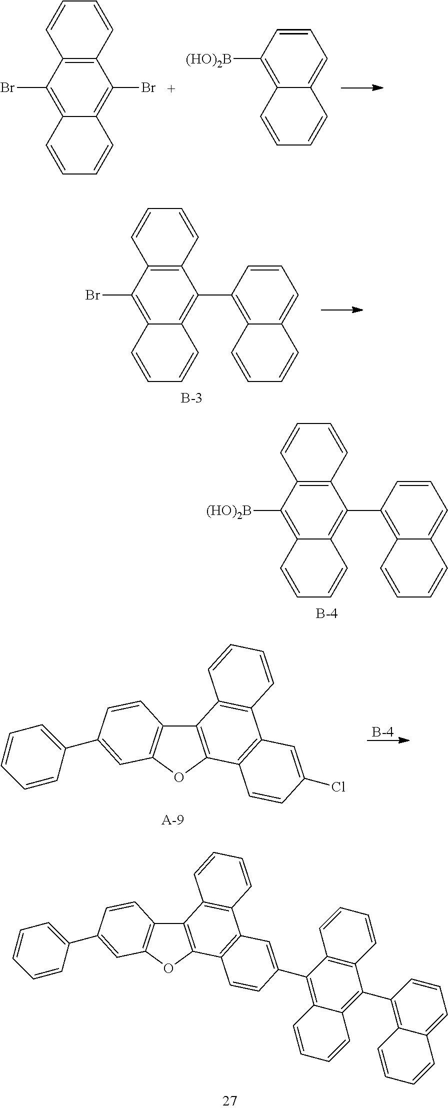

### Synthesis of Intermediate B-3

5.04 g (15 mmol) of 9,10-dibromoanthracene, 1.72 g (10 mmol) of naphthalene-1-boronic acid, 0.58 g (0.5 mmol) of Pd(PPh₃)₄, and 4.15 g (30 mmol) of potassium carbonate were dissolved in 200 ml of a THF:H₂O=2:1 (vol) solution, and reflux-stirred at 80° C. for 2 hours. Then, an organic layer was extracted therefrom three times by using 40 ml of H₂O and 40 ml of ethylether. The organic layer was dried using magnesium sulfate, and a solvent was removed therefrom by evaporation. The residue was separated and purified through a silica gel chromatography to obtain 3.06 g of Intermediate B-3 (yield: 80%). The compound thus obtained was confirmed by using LC-MS. (C₂₄H₁₅Br; M+ calcd: 383.29. found: 383.45).

### Synthesis of Intermediate B-4

3.83 g (10 mmol) of Intermediate B-3 was dissolved in 80 ml of a THF, and, at −78° C., 4 ml (10 mmol) of n-BuLi (2.5 M in hexane) was slowly and dropwisely added thereto. After stirring for 1 hour while maintaining the temperature at −78° C., 1.15 g (11 mmol) of trimethylborate was slowly and dropwisely added thereto, and then stirred for 6 hours at room temperature. When the reaction was completed, an organic layer was extracted therefrom three times by using 50 ml of H₂O, stirring, and 40 ml of ethylether. The organic layer was dried using magnesium sulfate, and a solvent was removed therefrom by evaporation. The residue was separated and purified through a silica gel chromatography to obtain 2.78 g of Intermediate B-4 (yield: 80%). The compound thus obtained was confirmed by using LC-MS. (C₂₄H₁₇BO₂; M+ calcd: 348.13. found: 348.54).

### Synthesis of Compound 27

3.79 g (10 mmol) of Intermediate A-9, 3.83 g (11 mmol) of Intermediate B-4, 0.58 g (0.5 mmol) of Pd(PPh₃)₄, and 4.15 g (30 mmol) of potassium carbonate were dissolved in 200 ml of a THF:H₂O=2:1 (vol) solution, and reflux-stirred at 80° C. for 4 hours. Then, an organic layer was extracted therefrom three times by using 40 ml of H₂O and 40 ml of ethylether. The organic layer was dried using magnesium sulfate, and a solvent was removed therefrom by evaporation. The residue was separated and purified through a silica gel chromatography to obtain 5.48 g of Compound 27 (yield: 85%). The compound thus obtained was confirmed by using LC-MS and ¹H NMR. (C₅₀H₃₀OSi; M+ calcd: 646.23. found: 646.47).

### Synthesis Example 5

### Synthesis of Compound 42

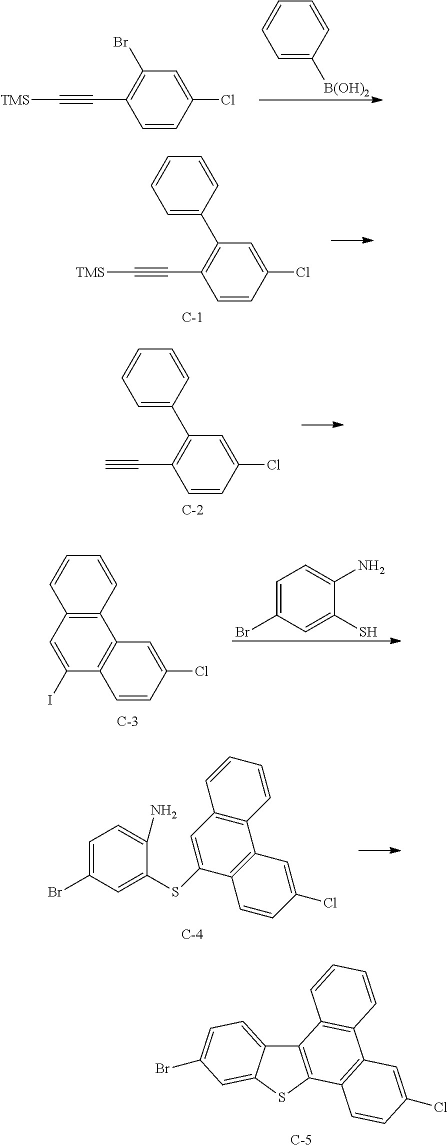

### Synthesis of Intermediate C-1

2.88 g (10 mmol) of ((2-bromo-4-chlorophenyl)ethynyl)trimethylsilane, 1.34 g (11 mmol) of phenylboronic acid, 0.58 g (0.5 mmol) of Pd(PPh₃)₄, and 4.15 g (30 mmol) of potassium carbonate were dissolved in 200 ml of a THF:H₂O=2:1 (vol) solution, and reflux-stirred at 80° C. for 5 hours. Next, the reaction mixture was cooled to room temperature, and then an organic layer was extracted therefrom three times by using 50 ml of H₂O and 50 ml of ethylether. The organic layer was dried using magnesium sulfate, and a solvent was removed therefrom by evaporation. The residue was separated and purified through a silica gel chromatography to obtain 2.62 g of Intermediate C-1 (yield: 72%). The compound thus obtained was confirmed by using LC-MS. (C₁₇H₁₇ClSi; M+ calcd: 284.08. found: 284.54).

### Synthesis of Intermediate C-2

2.84 g (10 mmol) of Intermediate C-1 and 4.15 g (30 mmol) of potassium carbonate were dissolved in 100 ml of methanol and stirred at room temperature for 3 hours. Then, the reaction solution was distilled under a reduced pressure, an organic layer was extracted therefrom three times by using 40 ml of H₂O and 40 ml of ethylether. The organic layer was dried using magnesium sulfate, and a solvent was removed therefrom by evaporation. The residue was separated and purified through a silica gel chromatography to obtain 2.02 g of Intermediate C-2 (yield: 95%). The compound thus obtained was confirmed by using LC-MS. (C₁₄H₉Cl; M+ calcd: 212.04. found: 212.54).

### Synthesis of Intermediate C-3

2.12 g (10 mmol) of Intermediate C-2 was sufficiently dissolved in 100 ml of methylenechloride and stirred in an ice bath while maintaining the temperature at 0° C. for 30 minutes. Next, 1.79 g (11 mmol) of iodine chloride was added thereto and additionally stirred for 30 minutes. Then, an organic layer was extracted from the reaction solution three times by using 100 ml of H₂O and 50 ml of ethylether. The organic layer was dried using magnesium sulfate, and a solvent was removed therefrom by evaporation. The residue was separated and purified through a silica gel chromatography to obtain 2.88 g of Intermediate C-3 (yield: 85%). The compound thus obtained was confirmed by using LC-MS. (C₁₄H₈ClI; M+ calcd: 337.94. found: 337.98).

### Synthesis of Intermediate C-4

3.38 g (10 mmol) of Intermediate C-3, 2.24 g (11 mmol) of 2-amino-5-bromobenzenethiol, and 4.15 g (30 mmol) potassium carbonate were dissolved in 80 ml of DMF and reflux-stirred at 150° C. for 48 hours. After cooling the reaction solution to room temperature, an organic layer was extracted from the reaction solution three times by using 50 ml of H₂O and 50 ml of ethylether. The organic layer was dried using magnesium sulfate, and a solvent was removed therefrom by evaporation. The residue was separated and purified through a silica gel chromatography to obtain 2.90 g of Intermediate C-4 (yield: 70%). The compound thus obtained was confirmed by using LC-MS. (C₂₀H₁₃BrClNS; M+ calcd: 412.96. found: 412.98).

### Synthesis of Intermediate C-5

826 mg (2 mmol) of Intermediate C-4, 1.73 g (25 mmol) of sodium nitrite, 0.8 mL of HCl, 8 ml of glacial acetic acid, and 1.2 ml of H₂O were stirred in a ice bath for 1 hour, and additionally stirred for 12 hours at room temperature. A temperature of the reaction solution was increased to 90° C., and then an aqueous solution including 1.59 g (10 mmol) of copper sulfate dissolved in 30 ml of water and 1.5 ml of acetic acid was dropwisely added thereto for 1 hour. Then, the mixture was additionally stirred for 30 minutes, cooled to room temperature, and an organic layer was extracted from the reaction solution four times with 20 ml of water and 20 ml of diethylether. The organic layer was dried using magnesium sulfate, and a solvent was removed therefrom by evaporation. The residue was separated and purified through a silica gel chromatography to obtain 713 mg of Intermediate C-5 (yield: 90%). The compound thus obtained was confirmed by using LC-MS. (C₂₀H₁₀BrClS; M+ calcd: 395.94. found: 395.98).

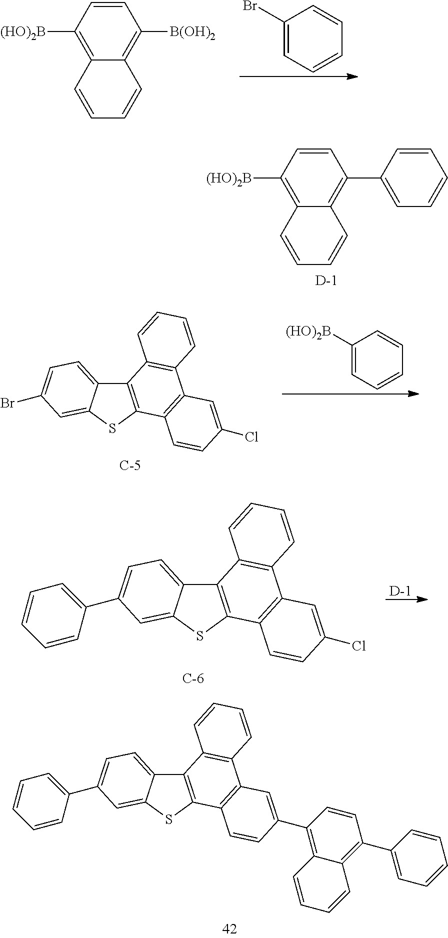

### Synthesis of Intermediate D-1

2.59 g (12 mmol) of naphthalene-1,4-diboronic acid, 1.57 g (10 mmol) of bromobenzene, 0.58 g (0.5 mmol) of Pd(PPh₃)₄, and 4.15 g (30 mmol) of potassium carbonate were dissolved in 200 ml of a THF:H₂O=2:1 (vol) solution, and reflux-stirred at 80° C. for 2 hours. Then, an organic layer was extracted therefrom three times by using 40 ml of H₂O and 40 ml of ethylether. The organic layer was dried using magnesium sulfate, and a solvent was removed therefrom by evaporation. The residue was separated and purified through a silica gel chromatography to obtain 1.74 g of Intermediate D-1 (yield: 70%). The compound thus obtained was confirmed by using LC-MS. (C₁₆H₁₃BO₂; M+ calcd: 248.10. found: 248.35).

### Synthesis of Intermediate C-6

3.98 g (10 mmol) of Intermediate C-5, 1.10 g (10 mmol) of phenylboronic acid, 0.58 g (0.5 mmol) of Pd(PPh₃)₄, and 4.15 g (30 mmol) of potassium carbonate were dissolved in 200 ml of a THF:H₂O=2:1 (vol) solution, and reflux-stirred at 80° C. for 4 hours. Then, an organic layer was extracted therefrom three times by using 40 ml of H₂O and 40 ml of ethylether. The organic layer was dried using magnesium sulfate, and a solvent was removed therefrom by evaporation. The residue was separated and purified through a silica gel chromatography to obtain 3.36 g of Intermediate C-6 (yield: 85%). The compound thus obtained was confirmed by using LC-MS. (C₂₆H₁₅ClS; M+ calcd: 394.06. found: 394.51).

### Synthesis of Compound 42

1.97 g (5 mmol) of Intermediate C-6, 1.24 g (5 mmol) of Intermediate D-1, 0.30 g (0.25 mmol) of Pd(PPh₃)₄, and 2.08 g (15 mmol) of potassium carbonate were dissolved in 100 ml of a THF:H₂O=2:1 (vol) solution, and reflux-stirred at 80° C. for 4 hours. Then, an organic layer was extracted therefrom three times by using 40 ml of H₂O and 40 ml of ethylether. The organic layer was dried using magnesium sulfate, and a solvent was removed therefrom by evaporation. The residue was separated and purified through a silica gel chromatography to obtain 2.25 g of Compound 42 (yield: 80%). The compound thus obtained was confirmed by using LC-MS and ¹H NMR. (C₄₂H₂₆S; M+ calcd: 562.18. found: 562.35).

### Synthesis Example 6

### Synthesis of Compound 44

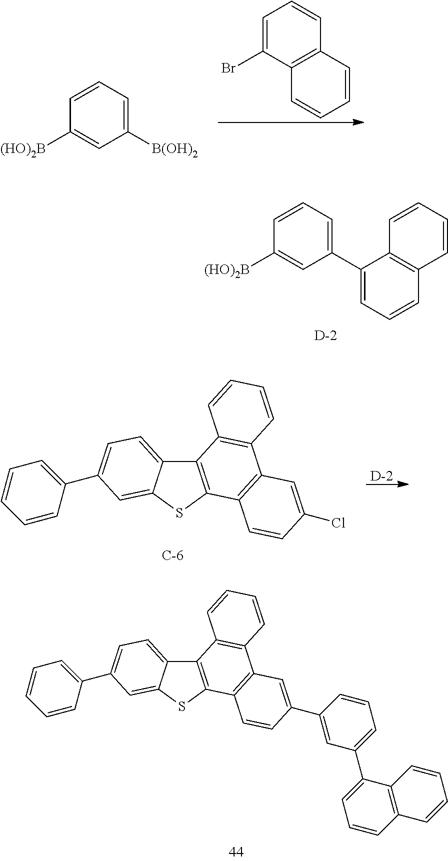

### Synthesis of Intermediate D-2

1.99 g (12 mmol) of 1,3-phenyldiboronic acid, 2.07 g (10 mmol) of 1-bromonaphthalene, 0.58 g (0.5 mmol) of Pd(PPh₃)₄, and 4.15 g (30 mmol) of potassium carbonate were dissolved in 100 ml of a THF:H₂O=2:1 (vol) solution, and reflux-stirred at 80° C. for 2 hours. Then, an organic layer was extracted therefrom three times by using 40 ml of H₂O and 40 ml of ethylether. The organic layer was dried using magnesium sulfate, and a solvent was removed therefrom by evaporation. The residue was separated and purified through a silica gel chromatography to obtain 1.74 g of Intermediate D-2 (yield: 70%). The compound thus obtained was confirmed by using LC-MS. (C₁₆H₁₃BO₂; M+ calcd: 248.10. found: 248.25).

### Synthesis of Compound 44

2.11 g of Compound 44 (yield: 75%) was synthesized in the same manner as in the synthesis method of Compound 42, except that 1.24 g (5 mmol) of Intermediate D-2 was used instead of 1.24 g (5 mmol) of Intermediate D-1 in the synthesis of Compound 42. The compound thus obtained was confirmed by using LC-MS and ¹H NMR. (C₄₂H₂₆S; M+ calcd: 562.18. found: 562.36).

### Synthesis Example 7

### Synthesis of Compound 58

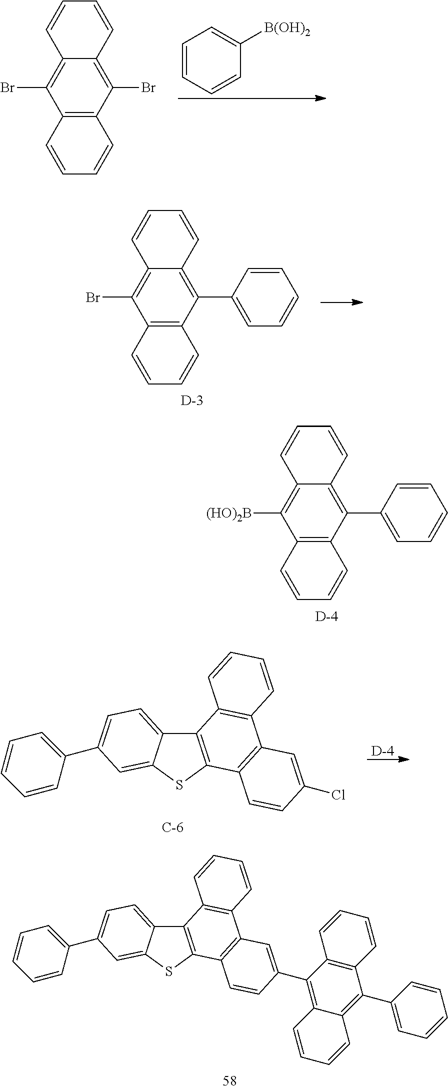

### Synthesis of Intermediate D-3

2.5 g of Intermediate D-3 (yield: 75%) was synthesized in the same manner as in the synthesis method of Intermediate B-3, except that 1.22 g (10 mmol) of phenylboronic acid was used instead of 1.72 g (10 mmol) of naphthalene-1-boronic acid in the synthesis of Intermediate B-3. The compound thus obtained was confirmed by using LC-MS. (C₂₀H₁₃Br; M+ calcd: 332.02. found: 332.54).

### Synthesis of Intermediate D-4

2.38 g of Intermediate D-4 (yield: 80%) was synthesized in the same manner as in the synthesis method of Intermediate B-4, except that 3.32 g (10 mmol) of Intermediate D-3 was used instead of 3.83 g (10 mmol) of Intermediate B-3 in the synthesis of Intermediate B-4. The compound thus obtained was confirmed by using LC-MS. (C₂₀H₁₅BO₂; M+ calcd: 298.12. found: 298.35).

### Synthesis of Compound 58

2.27 g of Compound 58 (yield: 74%) was synthesized in the same manner as in the synthesis method of Compound 42, except that 1.49 g (5 mmol) of Intermediate D-4 was used instead of 1.24 g (5 mmol) of Intermediate D-1 in the synthesis of Compound 42. The compound thus obtained was confirmed by using LC-MS and ¹H NMR. (C₄₆H₂₈S: M+ calcd: 612.19. found: 612.43).

### Synthesis Example 8

### Synthesis of Compound 64

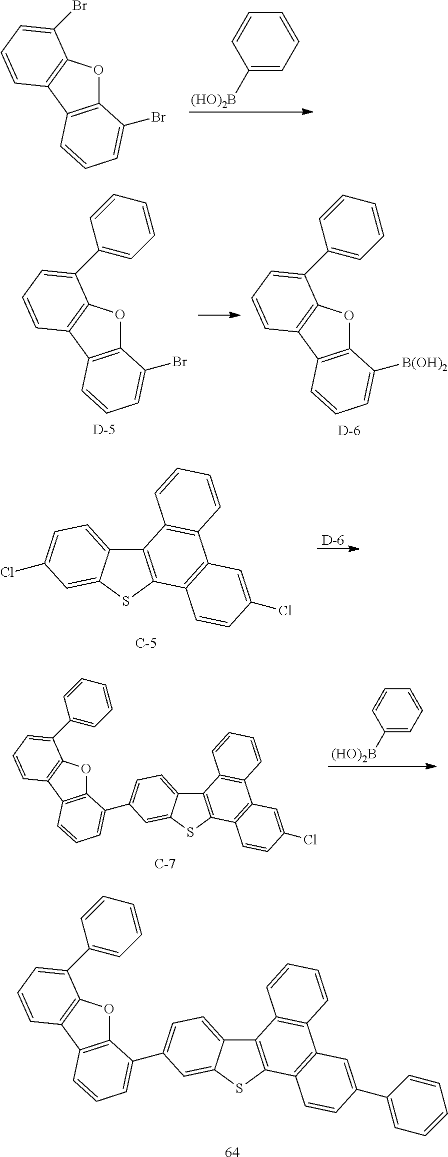

### Synthesis of Intermediate D-5

2.39 g of Intermediate D-5 (yield: 74%) was synthesized in the same manner as in the synthesis method of Intermediate B-3, except that 4.89 g (15 mmol) of 4,6-dibromodibenzo[b,d]furan was used instead of 5.04 g (15 mmol) of 9,10-dibromo anthracene in the synthesis of Intermediate B-3. The compound thus obtained was confirmed by using LC-MS. (C₂₀H₁₃Br; M+ calcd: 332.02. found: 332.54).

### Synthesis of Intermediate D-6

2.33 g of Intermediate D-6 (yield: 81%) was synthesized in the same manner as in the synthesis method of Intermediate B-4, except that 3.22 g (10 mmol) of Intermediate D-5 was used instead of 3.83 g (10 mmol) of Intermediate B-3 in the synthesis of Intermediate B-4. The compound thus obtained was confirmed by using LC-MS. (C₁₈H₁₃BO₃; M+ calcd: 288.10. found: 288.34).

### Synthesis of Intermediate C-7

4.71 g of Intermediate C-7 (yield: 84%) was synthesized in the same manner as in the synthesis method of Intermediate C-6, except that 2.88 g (10 mmol) of Intermediate D-6 was used instead of 1.10 g (10 mmol) of phenylboronic acid in the synthesis of Intermediate C-6. The compound thus obtained was confirmed by using LC-MS. (C₃₈H₂₁ClOS; M+ calcd: 560.10. found: 560.24).

### Synthesis of Compound 64

2.35 g of Compound 64 (yield: 78%) was synthesized in the same manner as in the synthesis method of Compound 42, except that Intermediate C-7 was used instead of Intermediate C-6, and phenylboronic acid was used instead of Intermediate D-1 in the synthesis of Compound 42. The compound thus obtained was confirmed by using LC-MS and ¹H NMR. (C₄₄H₂₆OS; M+ calcd: 602.17. found: 602.32).

Additional compounds were synthesized by using the same synthesis method with the synthesis pathways and appropriate intermediate materials, and the results of ¹H NMR and MS/FAB of the compounds are shown in Table 1.

Synthesizing other compounds than those shown in Table 1 by using a synthesis method with reference to the synthesis pathways and the raw materials above may be understood by one of ordinary skill in the art.

TABLE 1

| | | MS/FAB | |
|---|---|---|---|
| Compound | ¹H NMR (CDCl₃, 400 MHz) | found | Calc. |
| 3 | δ = 8.85-8.83 (d, 1H), 8.35-8.33 (d, 1H), 8.30-8.28 (d, | 470.41 | 470.57 |
| | 1H), 8.15-8.10 (dd, 2H), 8.08-8.03 (d, 1H), | | |
| | 8.01-7.99 (d, 1H), 7.96-7.82 (m, 4H), 7.77-7.75 (d, 1H), | | |
| | 7.72-7.66 (m, 7H), 7.58-7.53 (m, 2H), 7.42-7.38 (m, | | |
| | 1H) | | |
| 4 | δ = 8.92 (s, 1H), 8.90-8.88 (d, 1H), 8.62-8.60 (d, 1H), | 470.36 | 470.17 |
| | 8.47-8.45 (d, 1H), 8.35-8.33 (d, 1H), 8.17-8.15 (d, | | |
| | 1H), 8.08-8.06 (d, 1H), 7.88-7.56 (m, 7H), | | |
| | 7.50-7.42 (m, 6H), 7.38-7.36 (m, 1H), 7.32-7.30 (m, 1H) | | |
| 6 | δ = 8.92 (s, 1H), 8.92-8.90 (d, 1H), 8.68-8.66 (d, 1H), | 520.74 | 520.63 |
| | 8.57-8.55 (d, 1H), 8.46-8.44 (d, 1H), 8.24-8.22 (d, | | |
| | 1H), 8.17-8.15 (m, 2H), 7.88-7.84 (m, 5H), | | |
| | 7.66-7.62 (m, 3H), 7.48-7.44 (m, 4H), 7.40-7.38 (m, 2H), | | |
| | 7.34-7.32 (m, 2H) | | |
| 9 | δ = 8.90 (s, 1H), 8.85-8.83 (d, 1H), 8.72-8.70 (dd, | 546.87 | 546.67 |
| | 2H), 8.32-8.28 (m, 2H), 8.10-8.08 (d, 1H), | | |
| | 8.00-7.95 (m, 5H), 7.82-7.80 (m, 2H), 7.76-7.72 (m, 2H), | | |
| | 7.62-7.52 (m, 5H), 7.48-7.37 (m, 5H) | | |
| 12 | δ = 8.91 (s, 1H), 8.82-8.80 (d, 1H), 8.67-8.65 (dd, | 546.17 | 546.20 |
| | 2H), 8.45-8.43 (d, 2H), 8.36-8.33 (m, 2H), | | |
| | 8.22-8.20 (m, 1H), 7.96-7.88 (m, 7H), 7.74-7.68 (m, 5H), | | |
| | 7.52-7.50 (m, 1H), 7.36-7.34 (m, 1H) | | |
| 19 | δ = 8.88 (s, 1H), 8.72-8.70 (d, 2H), 8.65 (s, 1H), | 678.21 | 678.24 |
| | 8.42-8.40 (d, 1H), 8.24-8.22 (d, 1H), 8.05-8.03 (d, 1H), | | |
| | 7.82-7.78 (m, 8H), 7.70-7.68 (m, 2H), 7.54-7.42 (m, | | |
| | 8H), 7.28-7.18 (m, 9H) | | |
| 21 | δ = 8.88 (s, 1H), 8.75-8.73 (d, 1H), 8.70-8.68 (d, 1H), | 510.24 | 510.16 |
| | 8.38 (s, 1H), 8.32-8.30 (d, 1H), 8.25-8.23 (d, 1H), | | |
| | 7.94-7.92 (dd, 1H), 7.84-7.80 (m, 2H), 7.68-7.66 (d, | | |
| | 1H), 7.64-7.58 (m, 4H), 7.52-7.47 (m, 3H), | | |
| | 7.40-7.35 (m, 3H), 7.23-7.19 (m, 2H) | | |
| 27 | δ = 8.92 (s, 1H), 8.82-8.80 (d, 1H), 8.73-8.71 (d, 1H), | 646.47 | 646.23 |
| | 8.68-8.66 (s, 1H), 8.45-8.43 (d, 1H), 8.27-8.25 (d, | | |
| | 1H), 7.92-7.88 (m, 3H), 7.75-7.62 (m, 10H), | | |
| | 7.52-7.45 (m, 8H), 7.42-7.38 (m, 2H), 7.18-7.16 (m, 1H) | | |
| 29 | δ = 8.80 (d, 1H), 8.54-8.52 (d, 1H), 8.48-8.46 (d, 1H), | 596.25 | 596.21 |
| | 8.24 (s, 1H), 8.07-8.05 (d, 1H), 7.92-7.88 (m, 2H), | | |
| | 7.85-7.80 (m, 4H), 7.75-7.72 (m, 5H), 7.70-7.65 (m, | | |
| | 4H), 7.52-7.55 (m, 2H), 7.44-7.38 (m, 6H) | | |
| 31 | δ = 8.93 (s, 1H), 8.48-8.46 (d, 1H), 8.40-8.38 (d, 1H), | 646.75 | 646.23 |
| | 8.20 (d, 2H), 8.15-8.13 (d, 1H), 7.98-7.94 (m, 4H), | | |
| | 7.90-7.82 (m, 4H), 7.75-7.62 (m, 6H), 7.54-7.42 (m, | | |
| | 5H), 7.42-7.38 (m, 5H) | | |
| 39 | δ = 8.66-8.64 (d, 1H), 8.52-8.50 (d, 1H), 8.48 (s, 1H), | 536.47 | 536.16 |
| | 8.35-8.33 (d, 1H), 8.27-8.17 (d, 2H), 8.12-8.10 (m, | | |
| | 2H), 8.08-8.05 (m, 2H), 7.98-7.75 (m, 7H), | | |
| | 7.66-7.57 (m, 4H), 7.58-7.56 (m, 2H), 7.44-7.40 (m, 1H) | | |
| 42 | δ = 8.60-8.57 (m, 2H), 8.42 (s, 1H), 8.26-8.24 (d, 1H), | 562.35 | 562.18 |
| | 8.15-8.10 (d, 1H), 8.02-7.96 (m, 3H), 7.88-7.84 (m, | | |
| | 3H), 7.75-7.68 (m, 6H), 7.58-7.35 (m, 7H), | | |
| | 7.24-7.20 (m, 2H) | | |
| 44 | δ = 8.59-8.57 (d, 1H), 8.45-8.43 (d, 1H), 8.35 (s, 1H), | 562.36 | 562.18: |
| | 8.28-8.26 (d, 1H), 8.20 (s, 1H), 8.14-8.12 (d, 1H), | | |
| | 8.08-8.06 (d, 1H), 7.97-7.90 (m, 2H), 7.75-7.70 (m, | | |
| | 314), 7.66-7.54 (m, 8H), 7.42-7.36 (m, 4H), | | |
| | 7.24-7.22 (m, 1H), 7.10-7.08 (m, 1H) | | |
| 48 | δ = 8.52-8.50 (d, 1H), 8.42-8.40 (d, 1H), 8.38 (s, 1H), | 552.32 | 552.19 |
| | 8.28-8.25 (d, 1H), 8.24 (s, 1H), 8.17-8.10 (m, 2H), | | |
| | 7.84-7.80 (dd, 1H), 7.75-7.73 (d, 1H), 7.68-7.64 (d, | | |
| | 1H), 7.68-7.55 (m, 5H), 7.48-7.32 (m, 5H), | | |
| | 7.18-7.10 (m, 2H), 1.65 (s, 6H) | | |
| 54 | δ = 8.67-8.65 (d, 1H), 8.75-8.73 (d, 1H), | 553.45 | 553.19 |
| | 8.54-8.50 (m, 2H), 8.35-8.33 (d, 1H), 8.28-8.26 (d, 1H), | | |
| | 8.22-8.20 (m, 1H), 8.05-8.00 (m, 3H), 7.88-7.86 (dd, | | |
| | 1H), 7.80-7.78 (d, 1H), 7.72-7.69 (m, 1H), | | |
| | 7.64-7.48 (m, 3H), 7.38-7.32 (m, 3H), 7.24-7.20 (m, 2H), | | |
| | 1.64 (s, 6H) | | |
| 56 | δ = 8.72-8.64 (m, 3H), 8.55-8.52 (m, 2H), | 536.32 | 536.16 |
| | 8.34-8.14 (m, 3H), 8.10-8.08 (d, 1H), 7.88-7.84 (m, 3H), | | |
| | 7.72-7.62 (m, 4H), 7.54-7.42 (m, 6H), 7.28-7.24 (m, | | |
| | 2H) | | |
| 58 | δ = 8.70-8.68 (d, 1H), 8.62-8.60 (d, 1H), | 612.43 | 612.19 |
| | 8.53-8.50 (m, 2H), 8.28-8.26 (d, 1H), 8.10-8.08 (d, 1H), | | |
| | 7.88-7.84 (m, 2H), 7.78-7.70 (m, 5H), 7.64-7.58 (m, | | |
| | 4H), 7.52-7.49 (m, 3H), 7.41-7.30 (m, 8H) | | |
| 60 | δ = 8.72-8.70 (d, 1H), 8.58-8.56 (d, 1H), | 662.78 | 662.21 |
| | 8.48-8.42 (m, 2H), 8.28-8.26 (d, 1H), 8.15 (s, 1H), | | |
| | 8.04-8.02 (d, 1H), 7.98-7.92 (m, 4H), 7.84-7.80 (m, 3H), | | |
| | 7.74-7.70 (m, 2H), 7.64-7.58 (m, 6H), 7.56-7.50 (m, | | |
| | 3H), 7.47-7.38 (m, 5H) | | |
| 61 | δ = 8.78-8.75 (d, 1H), 8.62-8.60 (d, 1H), 8.34 (s, 1H), | 612.41 | 612.19 |
| | 8.30-8.25 (m, 2H), 8.17-8.15 (d, 1H), 8.10-8.08 (d, | | |
| | 1H), 7.88-7.84 (m, 3H), 7.78-7.74 (m, 4H), | | |
| | 7.72-7.68 (m, 5H), 7.60-7.56 (m, 2H), 7.48-7.28 (m, 7H) | | |
| 64 | δ = 8.83-8.80 (d, 1H), 8.58-8.56 (d, 1H), | 602.32 | 602.17 |
| | 8.38-8.34 (m, 2H), 8.28-8.26 (m, 1H), 8.18-8.16 (d, 1H), | | |
| | 8.14-8.12 (d, 1H) 7.90-7.86 (m, 3H), 7.80-7.76 (m, | | |
| | 3H), 7.72-7.68 (m, 2H), 7.58-7.50 (m, 4H), | | |
| | 7.48-7.46 (m, 1H), 7.37-7.30 (m, 5H), 7.28-7.26 (m, 1H) | | |

### Example 1

A indium tin oxide (ITO) glass substrate (available from Corning) having an ITO layer deposited thereon at a thickness of 15 Ω/cm2 (1200 Å) was cut to a size of 50 mm×50 mm×0.7 mm, sonicated in isopropyl alcohol and pure water for 5 minutes each, and then cleaned with UV and ozone for 30 minutes. The ITO glass substrate was then mounted on a vacuum depositor.

4,4′-Bis[N-phenyl-N-(9-phenylcarbazol-3-yl)amino]-1,1′-biphenyl (Compound 301) was deposited on the ITO glass substrate to form a hole injection layer having a thickness of 600 Å, N-[1,1′-biphenyl]-4-yl-9,9-dimethyl-N-[4-(9-phenyl-9H-carbazol-3-yl)phenyl]-9H-fluorene-2-amine (Compound 311) was deposited on the hole injection layer to form a hole transport layer having a thickness of 300 Å, and then Compound 47 (a host) and 4,4′-bis[2-(4-(N,N-diphenylamino)phenyl)vinyl]biphenyl (DPAVBi) (a dopant) were co-deposited at a weight ratio of 98:2 on the hole transport layer to form an emission layer having a thickness of 300 Å.

Alq₃ was deposited on the emission layer to form an electron transport layer having a thickness of 300 Å, LiF was deposited on the electron transport layer to form an electron injection layer having a thickness of 10 Å, and Al was deposited on the electrode injection layer to form a cathode having a thickness of 3,000 Å, thereby completing manufacture of an organic light-emitting device.

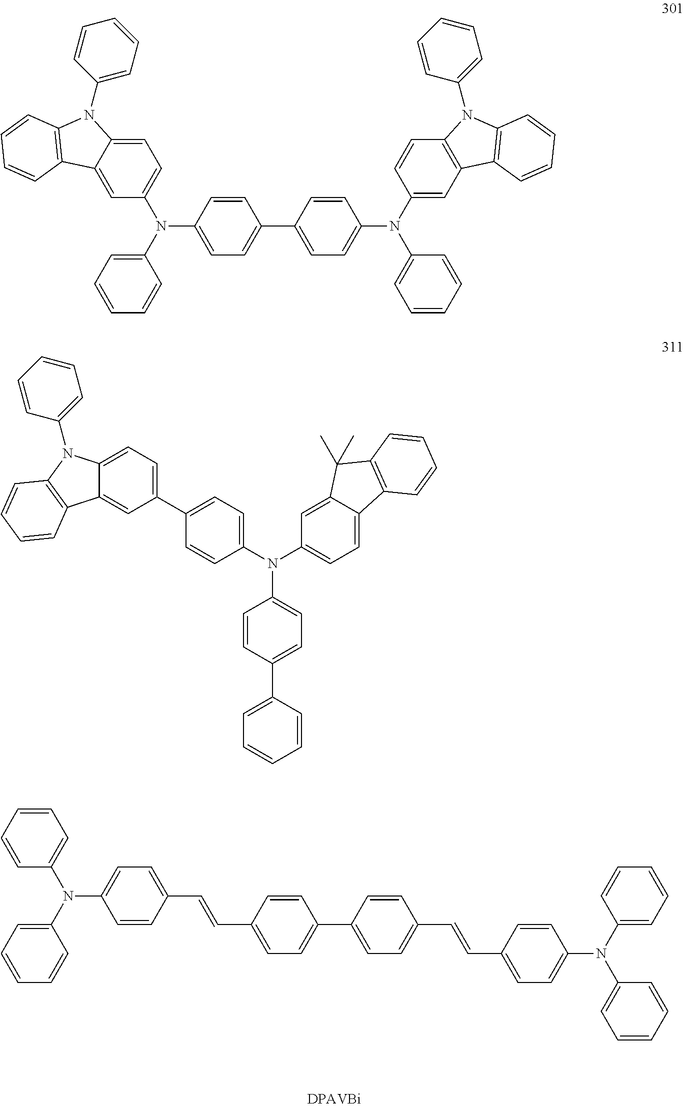 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; CC1(C)c2ccccc2-c2ccc(N(c3ccc(-c4ccccc4)cc3)c3ccc(-c4ccc5c(c4)c4ccccc4n5-c4ccccc4)cc3)cc21 ; C(=C\c1ccc(N(c2ccccc2)c2ccccc2)cc1)/c1ccc(-c2ccc(/C=C/c3ccc(N(c4ccccc4)c4ccccc4)cc3)cc2)cc1]

### Example 2

An organic light-emitting device was manufactured in the same manner as in Example 1, except that Compound 9 was used instead of Compound 3 as a host in the formation of the emission layer.

### Example 3

An organic light-emitting device was manufactured in the same manner as in Example 1, except that Compound 19 was used instead of Compound 3 as a host in the formation of the emission layer.

### Example 4

An organic light-emitting device was manufactured in the same manner as in Example 1, except that Compound 27 was used instead of Compound 3 as a host in the formation of the emission layer.

### Example 5

An organic light-emitting device was manufactured in the same manner as in Example 1, except that Compound 42 was used instead of Compound 3 as a host in the formation of the emission layer.

### Example 6

An organic light-emitting device was manufactured in the same manner as in Example 1, except that Compound 44 was used instead of Compound 3 as a host in the formation of the emission layer.

### Example 7

An organic light-emitting device was manufactured in the same manner as in Example 1, except that Compound 58 was used instead of Compound 3 as a host in the formation of the emission layer.

### Example 8

An organic light-emitting device was manufactured in the same manner as in Example 1, except that Compound 64 was used instead of Compound 3 as a host in the formation of the emission layer.

### Comparative Example 1

An organic light-emitting device was manufactured in the same manner as in Example 1, except that ADN was used instead of Compound 3 as a host in the formation of the emission layer.

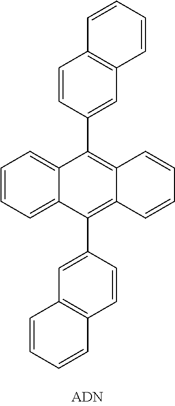 [SMILES: c1ccc2cc(-c3c4ccccc4c(-c4ccc5ccccc5c4)c4ccccc34)ccc2c1]

### Comparative Example 2

An organic light-emitting device was manufactured in the same manner as in Example 1, except that Compound A was used instead of Compound 3 as a host in the formation of the emission layer.

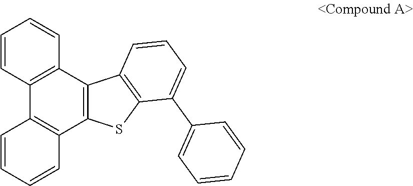 [SMILES: c1ccc(-c2cccc3c2sc2c4ccccc4c4ccccc4c32)cc1]

### Comparative Example 3

An organic light-emitting device was manufactured in the same manner as in Example 1, except that Compound B was used instead of Compound 3 as a host in the formation of the emission layer.

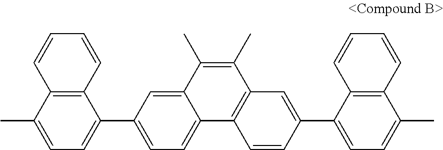

### Comparative Example 4

An organic light-emitting device was manufactured in the same manner as in Example 1, except that Compound C was used instead of Compound 3 as a host in the formation of the emission layer.

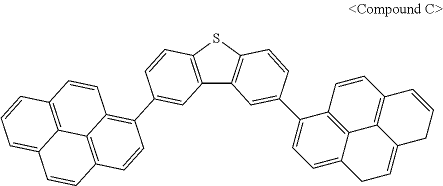 [SMILES: C1=Cc2ccc3c(-c4ccc5sc6ccc(-c7ccc8ccc9cccc%10ccc7c8c9%10)cc6c5c4)ccc4c3c2C(=CC4)C1]

### Evaluation Example 1

Driving voltages, current densities, brightnesses, efficiencies, and half-lives of the organic light-emitting devices prepared in Examples 1 to 8 and Comparative Examples 1 to 4 were evaluated by using Keithley SMU 236 and a luminance meter PR650. The results are shown in Table 2. The half-life is defined as the time for the brightness of an organic light-emitting device to decline to 50% of its initial brightness.

TABLE 2

| | | | | | | Color | |
|---|---|---|---|---|---|---|---|
| | | Driving | Current | | | of | Half life |
| | Emission layer | voltage | density | Brightness | Efficiency | emitted | (hr |
| | host | (V) | (mA/cm²) | (cd/m²) | (cd/A) | light | @100 mA/cm²) |
| Example 1 | Compound 3 | 6.58 | 50 | 3,240 | 6.48 | blue | 324 |
| Example 2 | Compound 9 | 6.25 | 50 | 3,340 | 6.68 | blue | 375 |
| Example 3 | Compound 19 | 6.37 | 50 | 3,325 | 6.65 | blue | 362 |
| Example 4 | Compound 27 | 6.24 | 50 | 3,410 | 6.82 | blue | 372 |
| Example 5 | Compound 42 | 6.28 | 50 | 3,390 | 6.78 | blue | 367 |
| Example 6 | Compound 44 | 6.25 | 50 | 3,285 | 6.57 | blue | 341 |
| Example 7 | Compound 58 | 6.46 | 50 | 3,310 | 6.62 | blue | 345 |
| Example 8 | Compound 64 | 6.32 | 50 | 3,375 | 6.75 | blue | 382 |
| Comparative | ADN | 7.35 | 50 | 2,065 | 4.13 | blue | 145 |
| Example 1 | | | | | | | |
| Comparative | Compound A | 6.30 | 50 | 2,450 | 4.90 | blue | 130 |
| Example 2 | | | | | | | |
| Comparative | Compound B | 6.85. | 50 | 2,370 | 4.74 | blue | 144 |
| Example 3 | | | | | | | |
| Comparative | Compound C | 7.24 | 50 | 2,425 | 4.85 | blue | 155 |
| Example 4 | | | | | | | |

As shown in Table 2, driving voltages, brightnesses, efficiencies, and half lives of the organic light-emitting devices prepared in Examples 1 to 8 were excellent compared to those of the organic light-emitting devices prepared in Comparative Examples 1 to 4.

As described above, according to the one or more of the above embodiments, an organic light-emitting device including the condensed cyclic compound may have a low driving voltage, high efficiency, high brightness, and a long lifespan.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope thereof as set forth in the following claims.

What is claimed is:

1. A condensed cyclic compound represented by Formula 1: 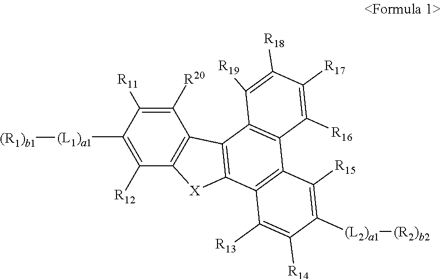
wherein, in Formula 1,
X is O or S;
L₁ and L₂ are each independently selected from a substituted or unsubstituted C₃-C₁₀ cycloalkylene group, a substituted or unsubstituted C₁-C₁₀ heterocycloalkylene group, a substituted or unsubstituted C₃-C₁₀ cycloalkenylene group, a substituted or unsubstituted C₁-C₁₀ heterocycloalkenylene group, a substituted or unsubstituted C₆-C₆₀ arylene group, a substituted or unsubstituted C₁-C₆₀ heteroarylene group, a substituted or unsubstituted divalent non-aromatic condensed polycyclic group, and a substituted or unsubstituted divalent non-aromatic condensed heteropolycyclic group;
a1 and a2 are each independently selected from 0, 1, 2, and 3, and when a1 is 2 or greater, a plurality of L₁ are identical to or different from each other, and when a2 is 2 or greater, a plurality of L₂ are identical to or different from each other;
R₁ and R₂ are each independently selected from a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —Si(Q₁)(Q₂)(Q₃), —B(Q₄)(Q₅), —N(Q₆)(Q₇), and —C(Q₈)=N(Q₉);
b1 and b2 are each independently an integer selected from 1 to 4, and when b1 is 2 or greater, a plurality of R₁ are identical to or different from each other, and when b2 is 2 or greater, a plurality of R₂ are identical to or different from each other;
R₁₁ to R₂₀ are each independently selected from a hydrogen, a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —Si(Q₁)(Q₂)(Q₃), —B(Q₄)(Q₅), and —N(Q₆)(Q₇);
at least one substituent of the substituted C₃-C₁₀ cycloalkylene group, substituted C₁-C₁₀ heterocycloalkylene group, substituted C₃-C₁₀ cycloalkenylene group, substituted C₁-C₁₀ heterocycloalkenylene group, substituted C₆-C₆₀ arylene group, substituted C₁-C₆₀ heteroarylene group, substituted a divalent non-aromatic condensed polycyclic group, substituted a divalent non-aromatic condensed heteropolycyclic group, substituted C₁-C₆₀ alkyl group, substituted C₂-C₆₀ alkenyl group, substituted C₂-C₆₀ alkynyl group, substituted C₁-C₆₀ alkoxy group, substituted C₃-C₁₀ cycloalkyl group, substituted C₁-C₁₀ heterocycloalkyl group, substituted C₃-C₁₀ cycloalkenyl group, substituted C₁-C₁₀ heterocycloalkenyl group, substituted C₆-C₆₀ aryl group, substituted C₆-C₆₀ aryloxy group, substituted C₆-C₆₀ arylthio group, substituted C₁-C₆₀ heteroaryl group, substituted monovalent non-aromatic condensed polycyclic group, and substituted monovalent non-aromatic condensed heteropolycyclic group is selected from:
a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, and a C₁-C₆₀ alkoxy group;
a C₁-C₆₀ alkyl group, a C₂-C₆₀ alkenyl group, a C₂-C₆₀ alkynyl group, and a C₁-C₆₀ alkoxy group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —Si(Q₁₁)(Q₁₂)(Q₁₃), —B(Q₁₄)(Q₁₅), and —N(Q₁₆)(Q₁₇);
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group;
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —Si(Q₂₁)(Q₂₂)(Q₂₃), —B(Q₂₄)(Q₂₅), and —N(Q₂₆)(Q₂₇); and
—Si(Q₃₁)(Q₃₂)(Q₃₃), —B(Q₃₄)(Q₃₅), and —N(Q₃₆)(Q₃₇),
wherein Q₁ to Q₉, Q₁₁ to Q₁₇, Q₂₁ to Q₂₇, and Q₃₁ to Q₃₇ are each independently selected from a hydrogen, a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group.

2. The condensed cyclic compound as claimed in claim 1, wherein, in Formula 1, L₁ and L₂ are each independently selected from,
a phenylene group, a pentalenylene group, an indenylene group, a naphthylene group, an azulenylene group, a heptalenylene group, an indacenylene group, an acenaphthylene group, a fluorenylene group, a spiro-fluorenylene group, a benzofluorenylene group, a dibenzofluorenylene group, a phenalenylene group, a phenanthrenylene group, an anthracenylene group, a fluoranthenylene group, a triphenylenylene group, a pyrenylene group, a chrysenylene group, a naphthacenylene group, a picenylene group, a perylenylene group, a pentaphenylene group, a hexacenylene group, a pentacenylene group, a rubicenylene group, a coronenylene group, an ovalenylene group, a pyrrolylene group, a thiophenylene group, a furanylene group, an imidazolylene group, a pyrazolylene group, a thiazolylene group, an isothiazolylene group, an oxazolylene group, a isoxazolylene group, a pyridinylene group, a pyrazinylene group, a pyrimidinylene group, a pyridazinylene group, an isoindolylene group, an indolylene group, an indazolylene group, a purinylene group, a quinolinylene group, an isoquinolinylene group, a benzoquinolinylene group, a phthalazinylene group, a naphthyridinylene group, a quinoxalinylene group, a quinazolinylene group, a cinnolinylene group, a carbazolylene group, a phenanthridinylene group, an acridinylene group, a phenanthrolinylene group, a phenazinylene group, a benzoimidazolylene group, a benzofuranylene group, a benzothiophenylene group, an isobenzothiazolylene group, a benzooxazolylene group, an isobenzooxazolylene group, a triazolylene group, a tetrazolylene group, an oxadiazolylene group, a triazinylene group, a dibenzofuranylene group, a dibenzothiophenylene group, a benzocarbazolylene group, a dibenzocarbazolylene group, a thiadiazolylene group, an imidazopyridinylene group, and an imidazopyrimidinylene group; and
a phenylene group, a pentalenylene group, an indenylene group, a naphthylene group, an azulenylene group, a heptalenylene group, an indacenylene group, an acenaphthylene group, a fluorenylene group, a spiro-fluorenylene group, a benzofluorenylene group, a dibenzofluorenylene group, a phenalenylene group, a phenanthrenylene group, an anthracenylene group, a fluoranthenylene group, a triphenylenylene group, a pyrenylene group, a chrysenylene group, a naphthacenylene group, a picenylene group, a perylenylene group, a pentaphenylene group, a hexacenylene group, a pentacenylene group, a rubicenylene group, a coronenylene group, an ovalenylene group, a pyrrolylene group, a thiophenylene group, a furanylene group, an imidazolylene group, a pyrazolylene group, a thiazolylene group, an isothiazolylene group, an oxazolylene group, a isoxazolylene group, a pyridinylene group, a pyrazinylene group, a pyrimidinylene group, a pyridazinylene group, an isoindolylene group, an indolylene group, an indazolylene group, a purinylene group, a quinolinylene group, an isoquinolinylene group, a benzoquinolinylene group, a phthalazinylene group, a naphthyridinylene group, a quinoxalinylene group, a quinazolinylene group, a cinnolinylene group, a carbazolylene group, a phenanthridinylene group, an acridinylene group, a phenanthrolinylene group, a phenazinylene group, a benzoimidazolylene group, a benzofuranylene group, a benzothiophenylene group, an isobenzothiazolylene group, a benzooxazolylene group, an isobenzooxazolylene group, a triazolylene group, a tetrazolylene group, an oxadiazolylene group, a triazinylene group, a dibenzofuranylene group, a dibenzothiophenylene group, a benzocarbazolylene group, a dibenzocarbazolylene group, a thiadiazolylene group, an imidazopyridinylene group, and an imidazopyrimidinylene group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzoxazolyl group, an isobenzooxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, a thiadiazolyl group, an imidazopyridinyl group, and an imidazopyrimidinyl group.

3. The condensed cyclic compound as claimed in claim 1, wherein, in Formula 1, L₁ and L₂ are each independently a group represented by one of Formulae 3-1 to 3-32 below: 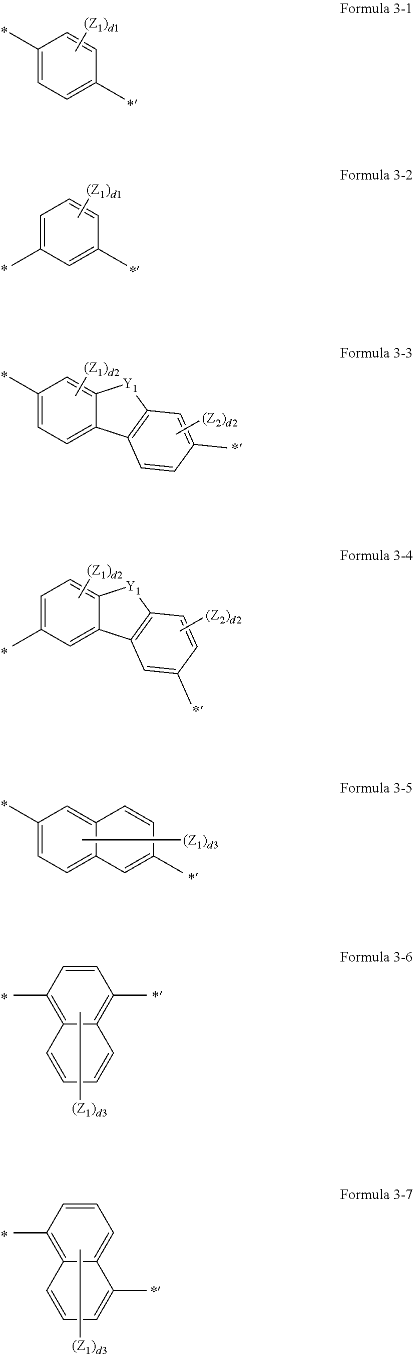 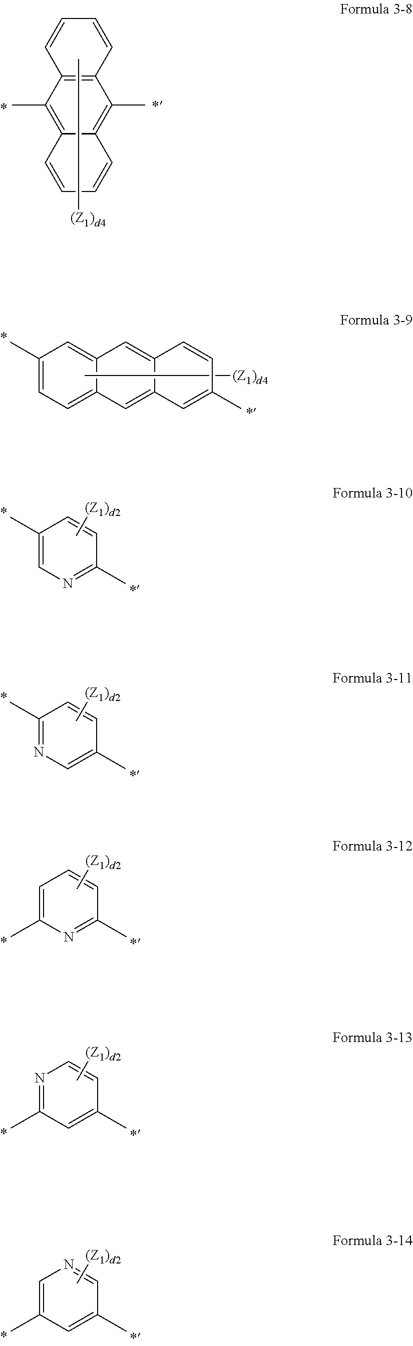 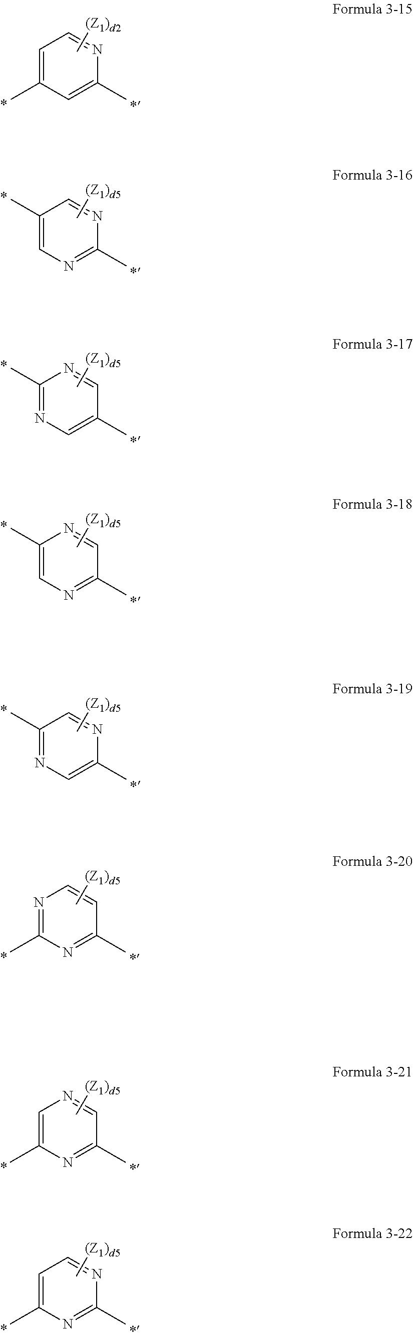 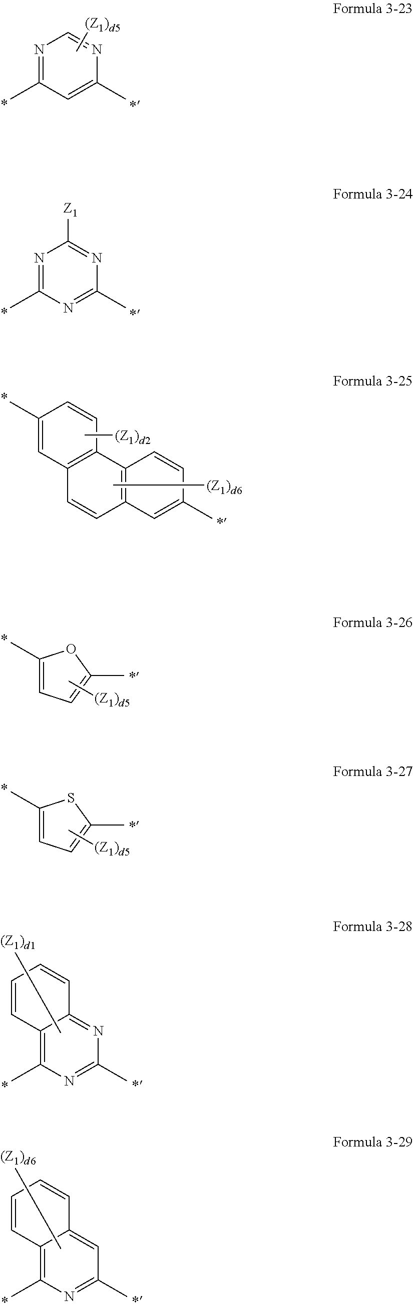 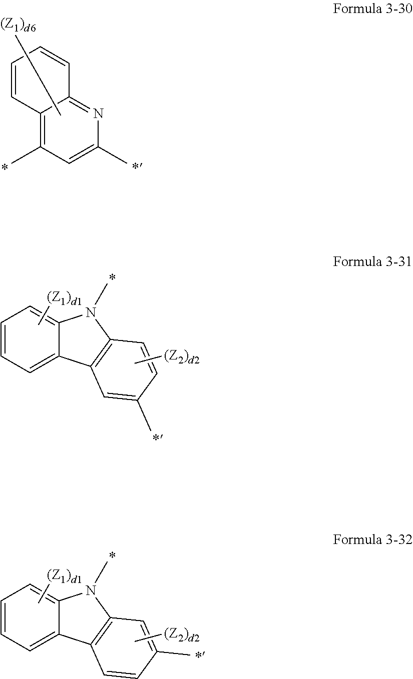
wherein, in Formulae 3-1 to 3-32,
Y₁ is O, S, C(Z₃)(Z₄), N(Z₅), or Si(Z₆)(Z₇);
Z₁ to Z₇ are each independently selected from a hydrogen, a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₁-C₂₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group;
d1 is an integer selected from 1 to 4,
d2 is an integer selected from 1 to 3,
d3 is an integer selected from 1 to 6,
d4 is an integer selected from 1 to 8,
d5 is an integer of 1 or 2,
d6 is an integer selected from 1 to 5, and
* and *′ are binding sites to a neighboring atom.

4. The condensed cyclic compound as claimed in claim 1, wherein L₁ and L₂ are each independently a group represented by one of Formulae 4-1 to 4-23 below: 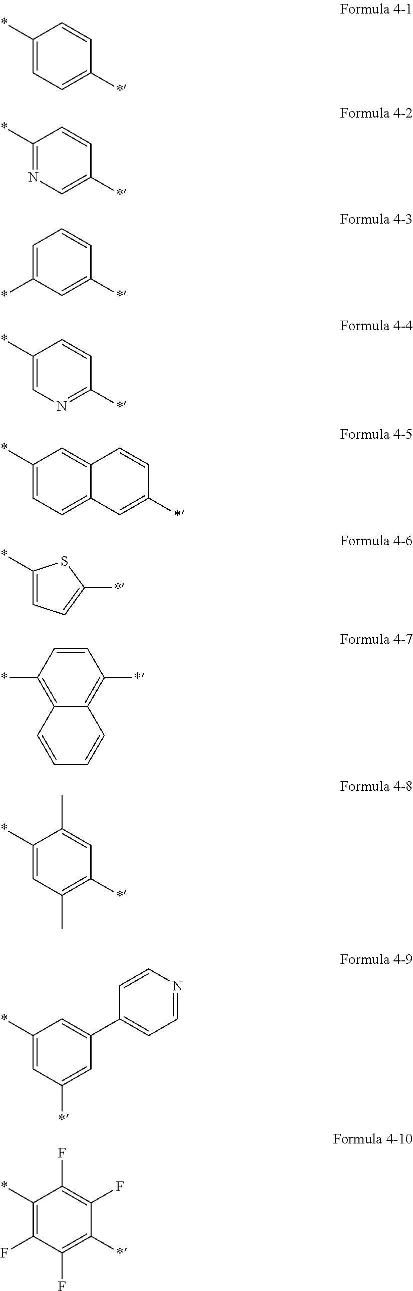 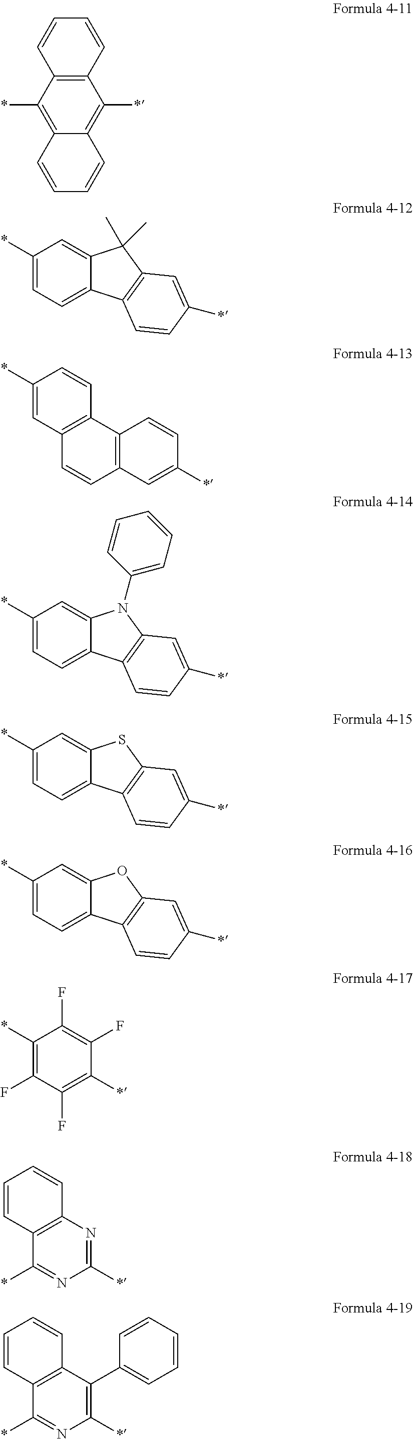 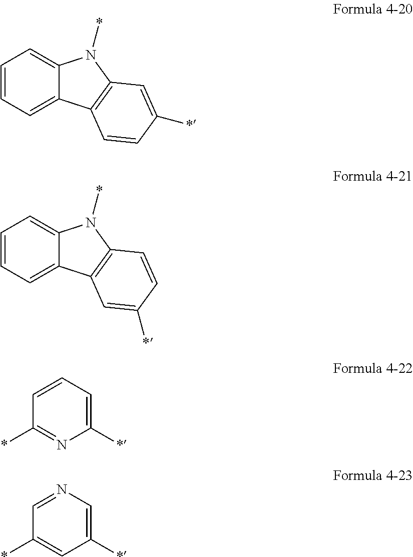
wherein, in Formulae 4-1 to 4-23, * and *′ are binding sites to a neighboring atom.

5. The condensed cyclic compound as claimed in claim 1, wherein, in Formula 1, a1 and a2 are each independently an integer of 0 or 1.

6. The condensed cyclic compound as claimed in claim 1, wherein, in Formula 1, R₁ and R₂ are each independently selected from:
a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzoxazolyl group, an isobenzooxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, a thiadiazolyl group, an imidazopyridinyl group, and an imidazopyrimidinyl group; and
a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzoxazolyl group, an isobenzooxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, a thiadiazolyl group, an imidazopyridinyl group, and an imidazopyrimidinyl group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzoxazolyl group, an isobenzooxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, a thiadiazolyl group, an imidazopyridinyl group, an imidazopyrimidinyl group, and —Si(Q₃₁)(Q₃₂)(Q₃₃); and
—Si(Q₁)(Q₂)(Q₃) and —C(Q₈)=N(Q₉);
wherein Q₁ to Q₃, Q₈, Q₉, and Q₃₁ to Q₃₃ are each independently selected from a hydrogen, a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₁-C₂₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group.

7. The condensed cyclic compound as claimed in claim 1, wherein, in Formula 1, R₁ and R₂ are each independently selected from:
a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, a triazinyl group, a dibenzofuranyl group, a dibenzothiophenyl group, a benzocarbazolyl group, and a dibenzocarbazolyl group;
a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, a triazinyl group, a dibenzofuranyl group, a dibenzothiophenyl group, a benzocarbazolyl group, and a dibenzocarbazolyl group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, C₁-C₂₀ alkyl group, C₁-C₂₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, a triazinyl group, a dibenzofuranyl group, a dibenzothiophenyl group, a benzocarbazolyl group, a dibenzocarbazolyl group, and —Si(Q₃₁)(Q₃₂)(Q₃₃); and
—Si(Q₁)(Q₂)(Q₃) and —C(Q₈)=N(Q₉), and
wherein Q₁ to Q₃, Q₈, Q₉, and Q₃₁ to Q₃₃ are each independently selected from a hydrogen, a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₁-C₂₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group.

8. The condensed cyclic compound as claimed in claim 1, wherein, in Formula 1, R₁ and R₂ are each independently a group represented by one of *—Si(Q₁)(Q₂)(Q₃), *—C(Q₈)=N(Q₉), and Formulae 5-1 to 5-14 below: 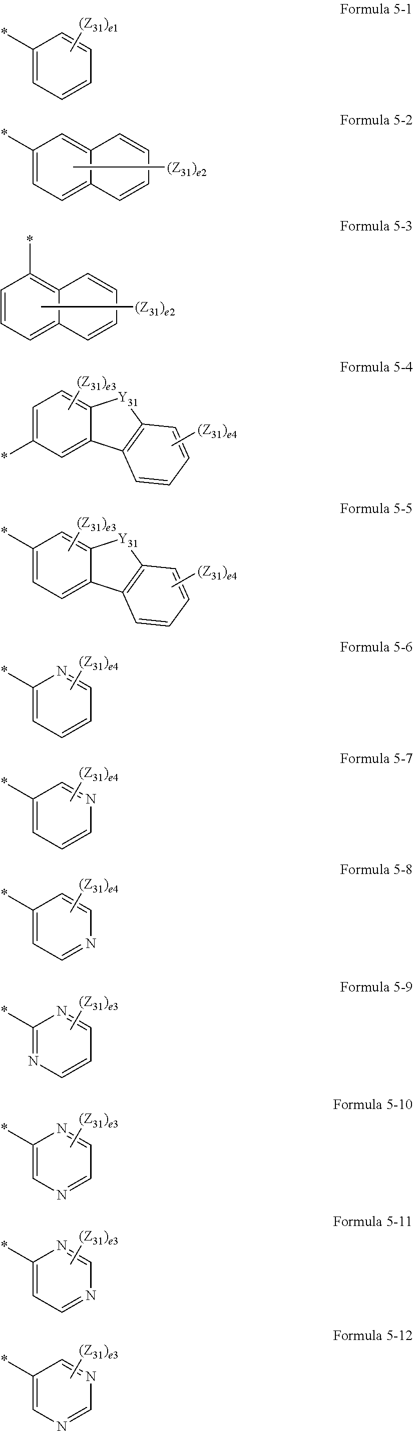 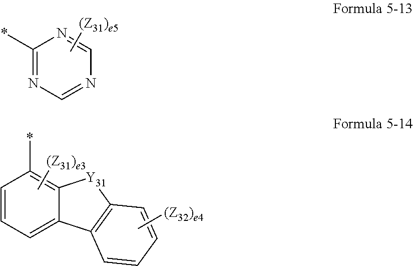
wherein, in Formulae 5-1 to 5-14,
Y₃₁ is O, S, C(Z₃₃)(Z₃₄), or N(Z₃₅);
Z₃₁ to Z₃₅ are each independently selected from,
a hydrogen, a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, and a C₁-C₂₀ alkoxy group;
a C₁-C₂₀ alkyl group and a C₁-C₂₀ alkoxy group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, and a phosphoric acid or a salt thereof;
a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, a triazinyl group, a dibenzofuranyl group, a dibenzothiophenyl group, a benzocarbazolyl group, and a dibenzocarbazolyl group;
a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, a triazinyl group, a dibenzofuranyl group, a dibenzothiophenyl group, a benzocarbazolyl group, and a dibenzocarbazolyl group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₁-C₂₀ alkoxy group, a phenyl group, and a naphthyl group; and
—Si(Q₃₁)(Q₃₂)(Q₃₃), and
wherein Q₁ to Q₃, Q₈, Q₉, and Q₃₁ to Q₃₃ are each independently selected from a hydrogen, a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₁-C₂₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group,
e1 is an integer selected from 1 to 5,
e2 is an integer selected from 1 to 7,
e3 is an integer selected from 1 to 3,
e4 is an integer selected from 1 to 4,
e5 is an integer of 1 or 2, and
* is a binding site to a neighboring atom.

9. The condensed cyclic compound as claimed in claim 1, wherein, in Formula 1, R₁ and R₂ are each independently a group represented by one of *—Si(Me)₃, *—Si(Ph)₃, *—CH═NH, and Formulae 6-1 to 6-25 below: 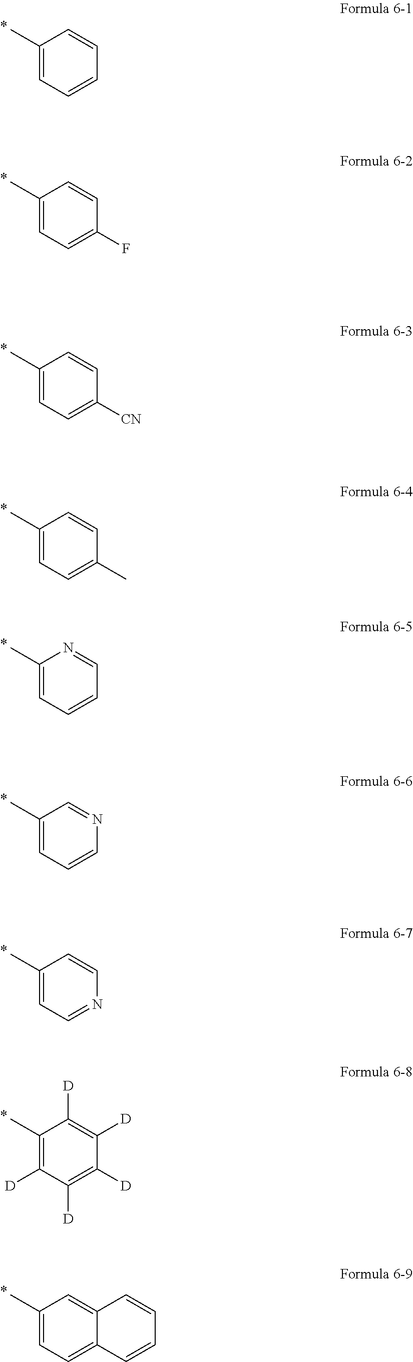 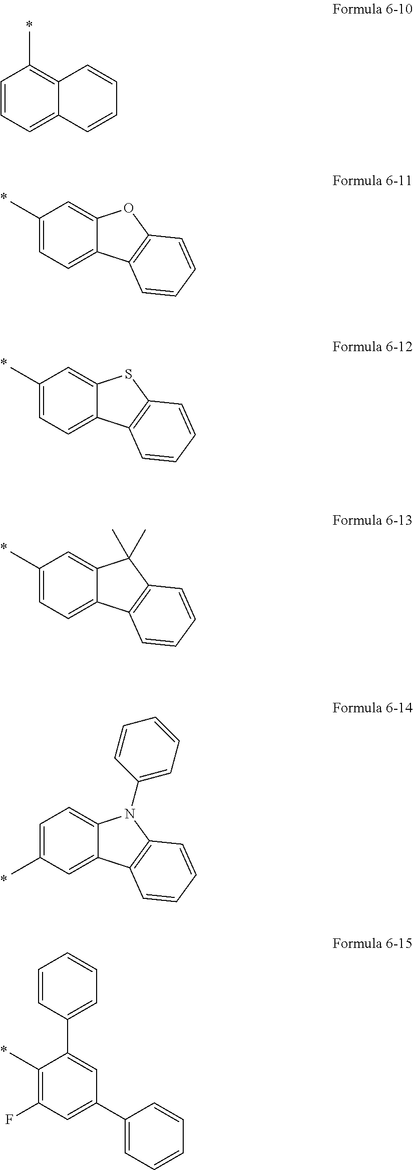 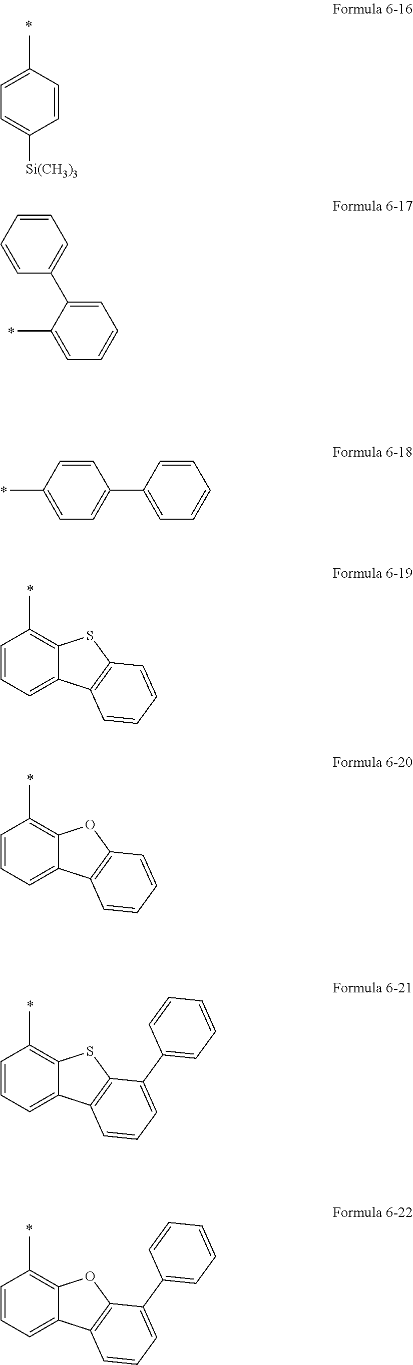 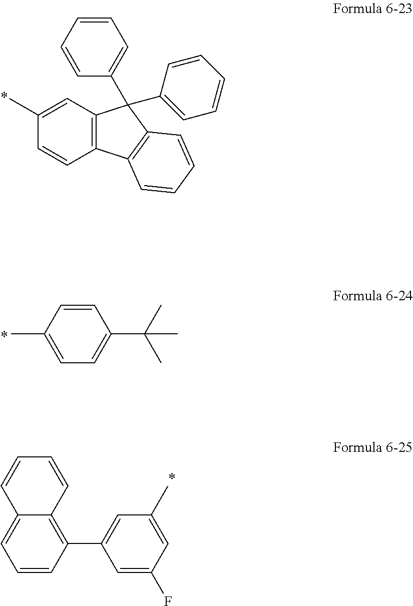
wherein, in Formulae 6-1 to 6-25, * is a binding site to a neighboring atom.

10. The condensed cyclic compound as claimed in claim 1, wherein, in Formula 1, R₁₁ to R₂₀ are each independently selected from a hydrogen, a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a substituted or unsubstituted C₁-C₂₀ alkyl group, a substituted or unsubstituted C₁-C₂₀ alkoxy group, a substituted or unsubstituted C₆-C₂₀ aryl group, a substituted or unsubstituted C₁-C₂₀ heteroaryl group, a substituted or unsubstituted monovalent non-aromatic condensed polycyclic group, a substituted or unsubstituted monovalent non-aromatic condensed heteropolycyclic group, and —Si(Q₁)(Q₂)(Q₃).

11. The condensed cyclic compound as claimed in claim 1, wherein, in Formula 1, R₁₁ to R₂₀ are each independently selected from a hydrogen, a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a substituted or unsubstituted C₁-C₂₀ alkyl group, and a substituted or unsubstituted C₁-C₂₀ alkoxy group.

12. The condensed cyclic compound as claimed in claim 1, wherein, the condensed cyclic compound is represented by one of Formulae 1-1 to 1-4: 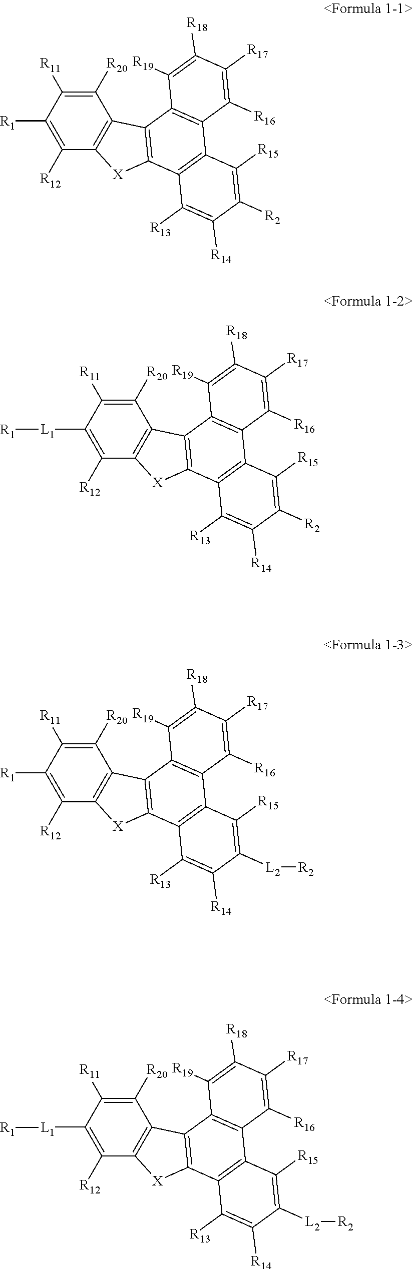
wherein, in Formulae 1-1 to 1-4,
X, L₁, L₂, R₁, R₂, and R₁₁ to R₂₀ are the same as defined with respect to Formula 1.

13. The condensed cyclic compound as claimed in claim 12, wherein, L₁ and L₂ are each independently a group represented by one of Formulae 4-1 to 4-23 below: 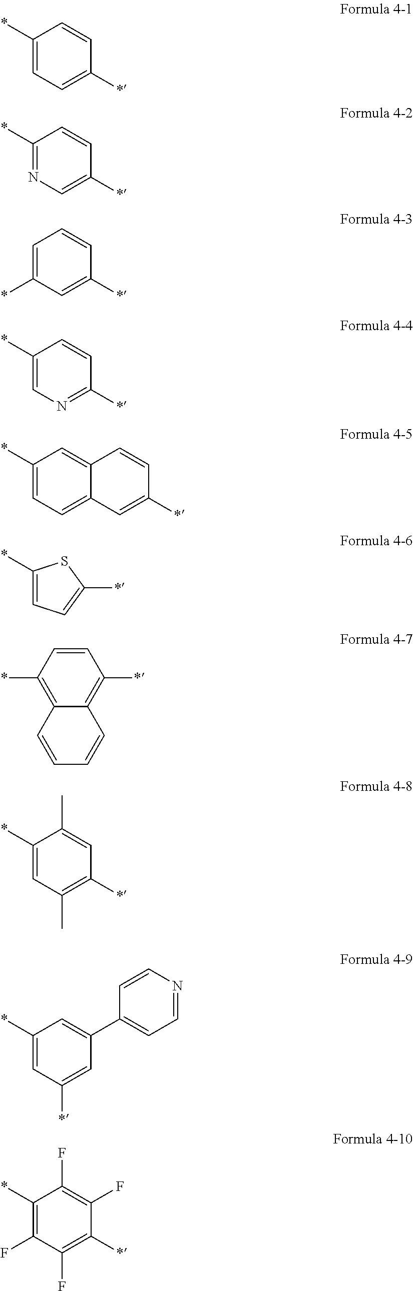 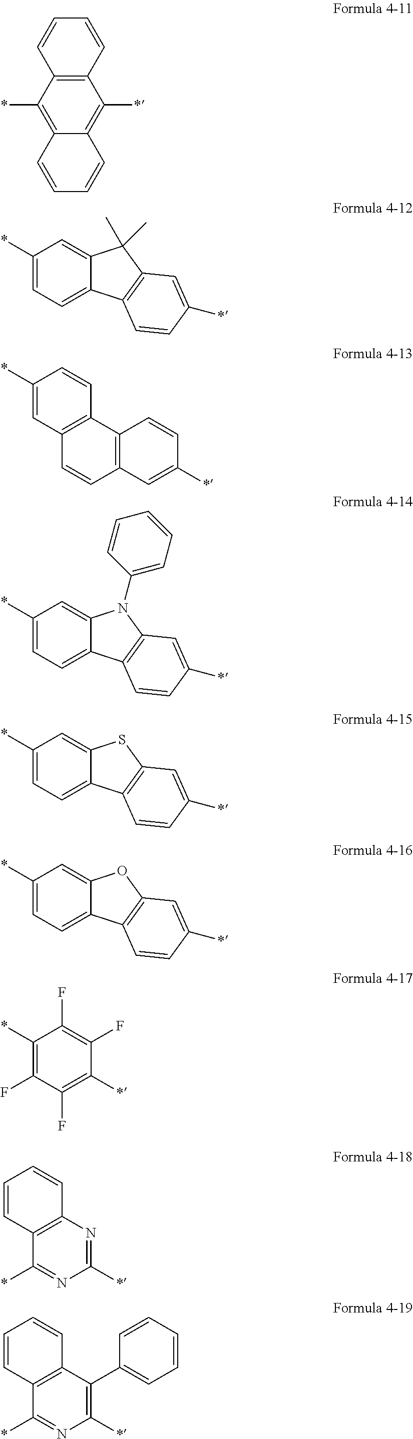 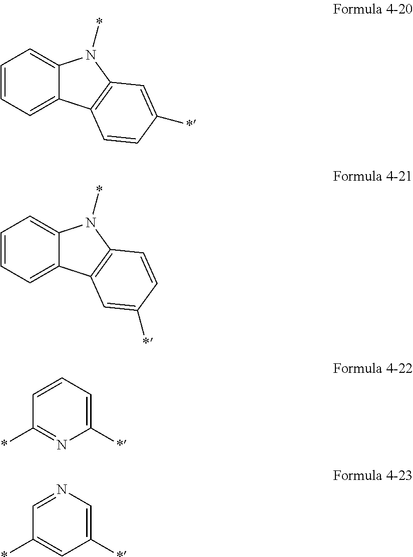
wherein, in Formulae 4-1 to 4-23, * and *′ are binding sites to a neighboring atom.

14. The condensed cyclic compound as claimed in claim 12, wherein R₁ and R₂ are each independently selected from a group represented by one of *—Si(Me)₃, *—Si(Ph)₃, *—CH═NH, and Formulae 6-1 to 6-25 below: 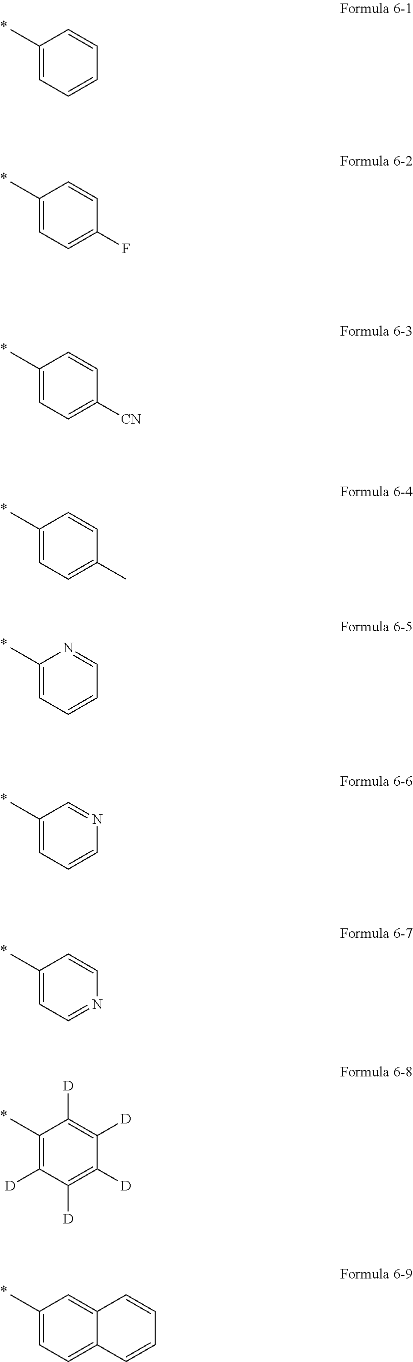 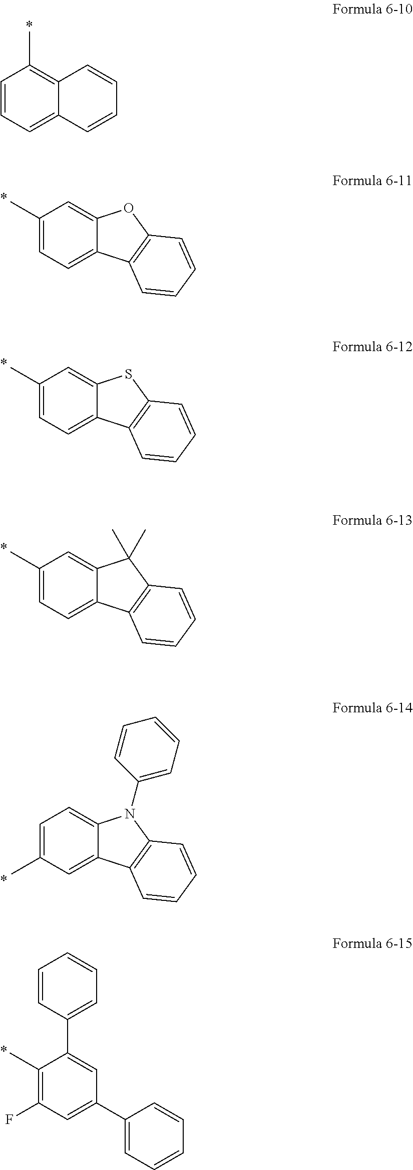 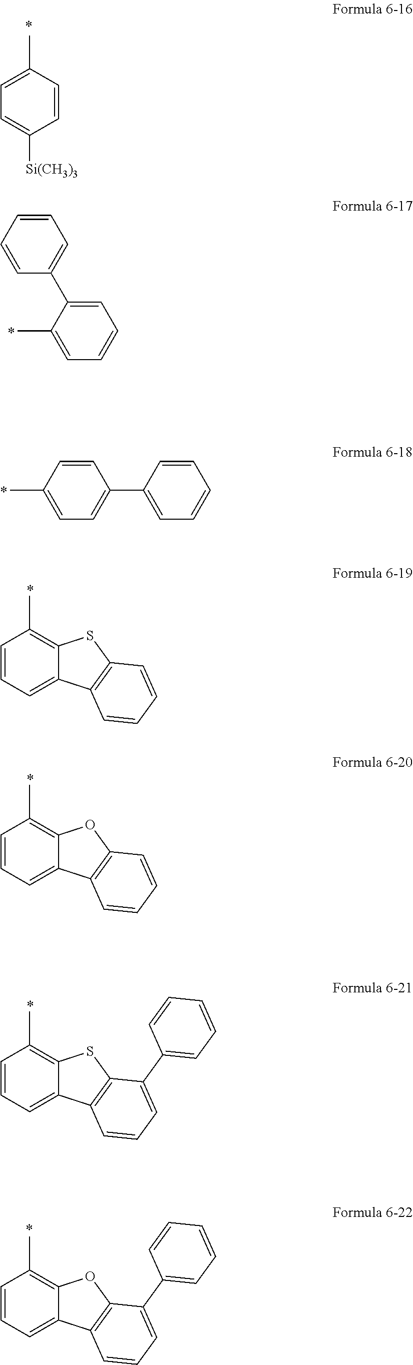 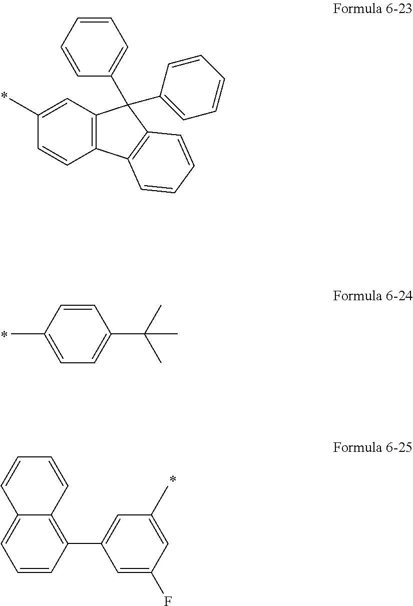
wherein, in Formulae 6-1 to 6-25, * is a binding site to a neighboring atom.

15. The condensed cyclic compound as claimed in claim 12, wherein R₁₁ to R₂₀ are each independently selected from a hydrogen, a deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a substituted or unsubstituted C₁-C₂₀ alkyl group, and a substituted or unsubstituted C₁-C₂₀ alkoxy group.

16. The condensed cyclic compound as claimed in claim 1, wherein the condensed cyclic compound is one of Compounds 1 to 64 below:

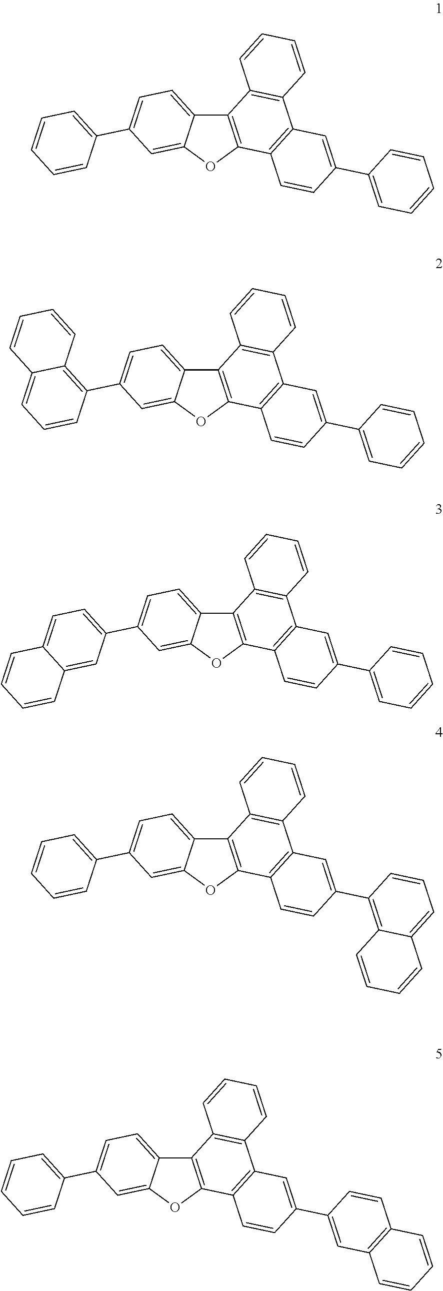 [SMILES: c1ccc(-c2ccc3c(c2)oc2c4ccc(-c5ccccc5)cc4c4ccccc4c32)cc1 ; c1ccc(-c2ccc3c(c2)c2ccccc2c2c4ccc(-c5cccc6ccccc56)cc4oc32)cc1 ; c1ccc(-c2ccc3c(c2)c2ccccc2c2c4ccc(-c5ccc6ccccc6c5)cc4oc32)cc1 ; c1ccc(-c2ccc3c(c2)oc2c4ccc(-c5cccc6ccccc56)cc4c4ccccc4c32)cc1 ; c1ccc(-c2ccc3c(c2)oc2c4ccc(-c5ccc6ccccc6c5)cc4c4ccccc4c32)cc1]

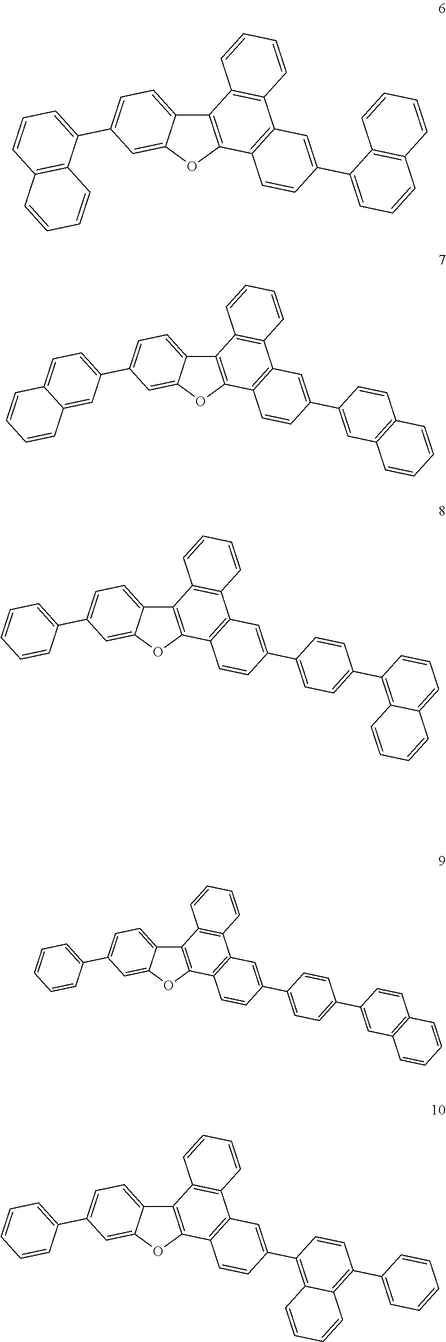 [SMILES: c1ccc2c(-c3ccc4c(c3)oc3c5ccc(-c6cccc7ccccc67)cc5c5ccccc5c43)cccc2c1 ; c1ccc2cc(-c3ccc4c(c3)oc3c5ccc(-c6ccc7ccccc7c6)cc5c5ccccc5c43)ccc2c1 ; c1ccc(-c2ccc3c(c2)oc2c4ccc(-c5ccc(-c6cccc7ccccc67)cc5)cc4c4ccccc4c32)cc1 ; c1ccc(-c2ccc3c(c2)oc2c4ccc(-c5ccc(-c6ccc7ccccc7c6)cc5)cc4c4ccccc4c32)cc1 ; c1ccc(-c2ccc3c(c2)oc2c4ccc(-c5ccc(-c6ccccc6)c6ccccc56)cc4c4ccccc4c32)cc1]

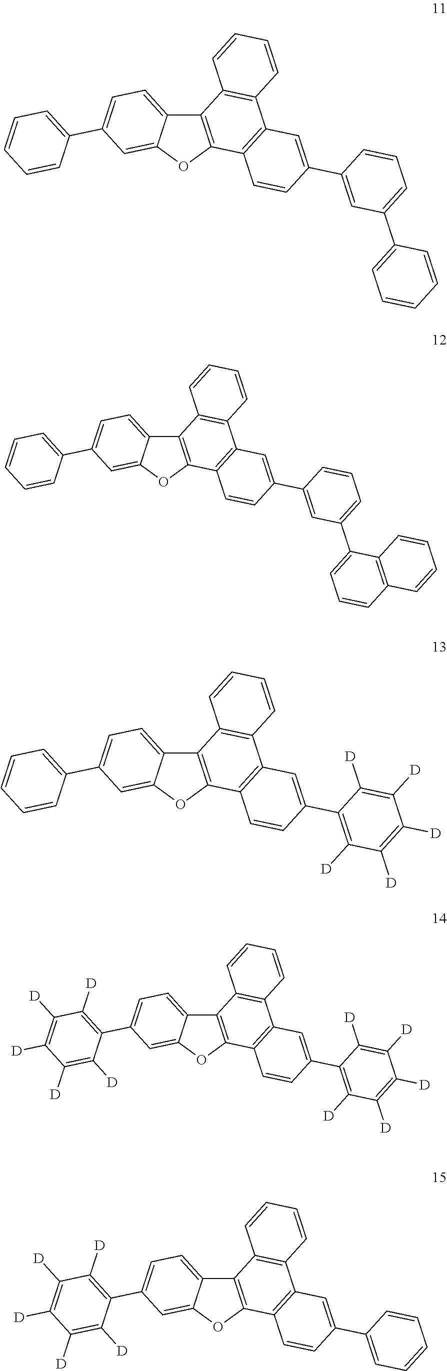 [SMILES: c1ccc(-c2cccc(-c3ccc4c(c3)c3ccccc3c3c5ccc(-c6ccccc6)cc5oc43)c2)cc1 ; c1ccc(-c2ccc3c(c2)oc2c4ccc(-c5cccc(-c6cccc7ccccc67)c5)cc4c4ccccc4c32)cc1 ; [2H]c1c([2H])c([2H])c(-c2ccc3c(c2)c2ccccc2c2c4ccc(-c5ccccc5)cc4oc32)c([2H])c1[2H] ; [2H]c1c([2H])c([2H])c(-c2ccc3c(c2)oc2c4ccc(-c5c([2H])c([2H])c([2H])c([2H])c5[2H])cc4c4ccccc4c32)c([2H])c1[2H] ; [2H]c1c([2H])c([2H])c(-c2ccc3c(c2)oc2c4ccc(-c5ccccc5)cc4c4ccccc4c32)c([2H])c1[2H]]

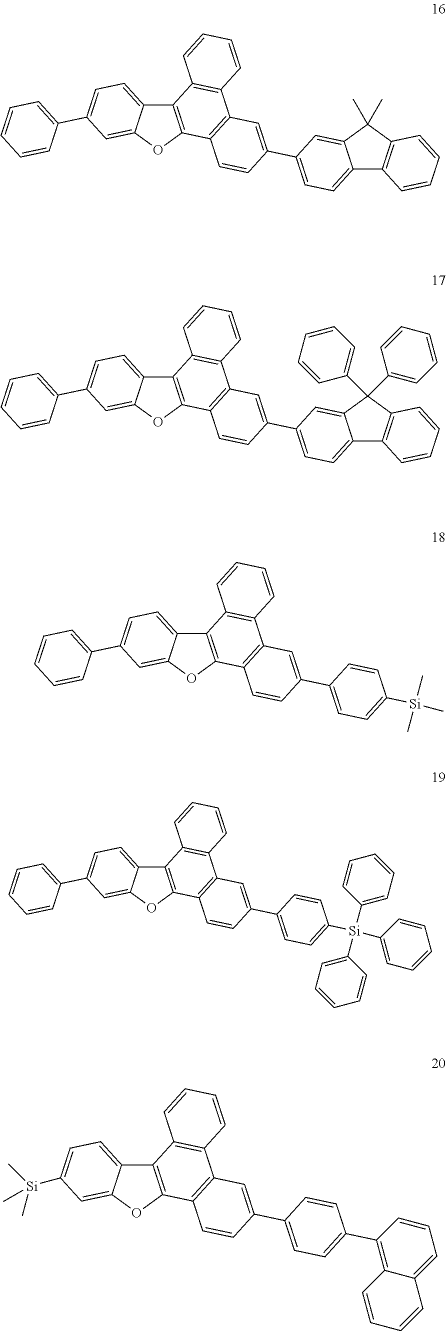

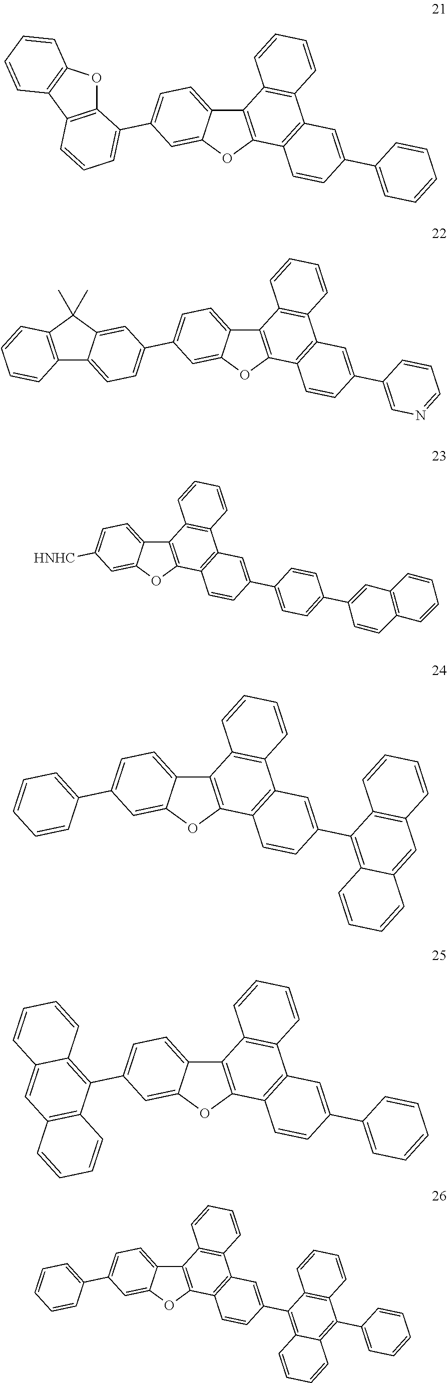

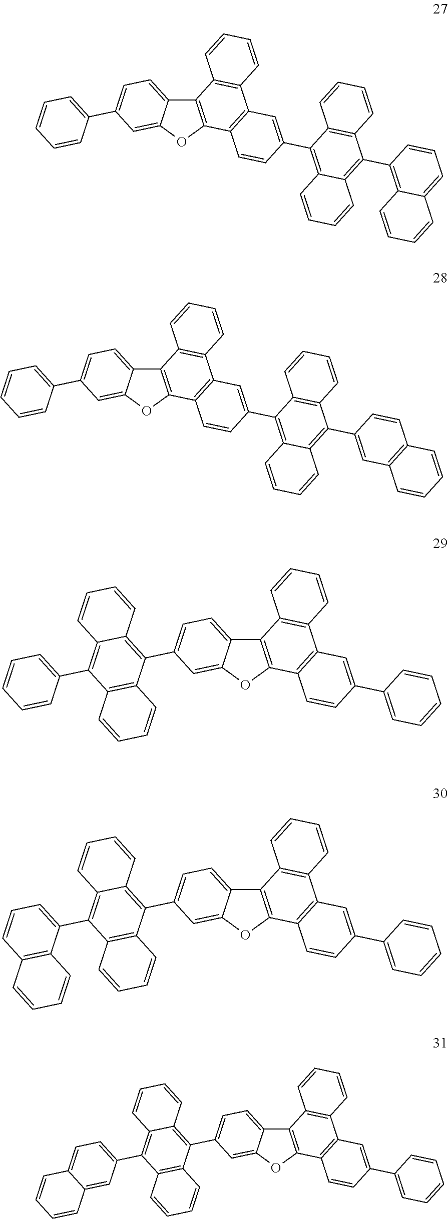 [SMILES: c1ccc(-c2ccc3c(c2)oc2c4ccc(-c5c6ccccc6c(-c6cccc7ccccc67)c6ccccc56)cc4c4ccccc4c32)cc1 ; c1ccc(-c2ccc3c(c2)oc2c4ccc(-c5c6ccccc6c(-c6ccc7ccccc7c6)c6ccccc56)cc4c4ccccc4c32)cc1 ; c1ccc(-c2ccc3c(c2)c2ccccc2c2c4ccc(-c5c6ccccc6c(-c6ccccc6)c6ccccc56)cc4oc32)cc1 ; c1ccc(-c2ccc3c(c2)c2ccccc2c2c4ccc(-c5c6ccccc6c(-c6cccc7ccccc67)c6ccccc56)cc4oc32)cc1 ; c1ccc(-c2ccc3c(c2)c2ccccc2c2c4ccc(-c5c6ccccc6c(-c6ccc7ccccc7c6)c6ccccc56)cc4oc32)cc1]

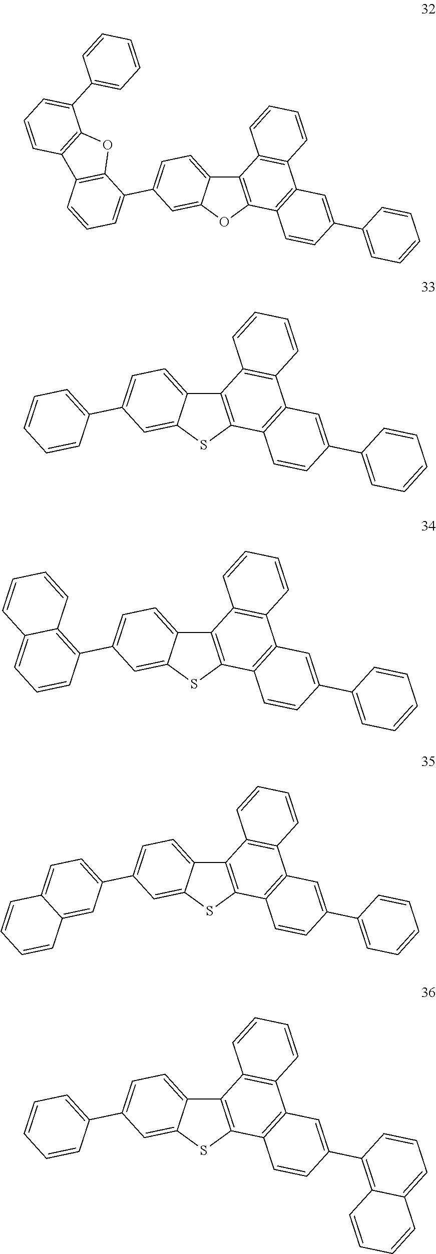 [SMILES: c1ccc(-c2ccc3c(c2)c2ccccc2c2c4ccc(-c5cccc6c5oc5c(-c7ccccc7)cccc56)cc4oc32)cc1 ; c1ccc(-c2ccc3c(c2)sc2c4ccc(-c5ccccc5)cc4c4ccccc4c32)cc1 ; c1ccc(-c2ccc3c(c2)c2ccccc2c2c4ccc(-c5cccc6ccccc56)cc4sc32)cc1 ; c1ccc(-c2ccc3c(c2)c2ccccc2c2c4ccc(-c5ccc6ccccc6c5)cc4sc32)cc1 ; c1ccc(-c2ccc3c(c2)sc2c4ccc(-c5cccc6ccccc56)cc4c4ccccc4c32)cc1]

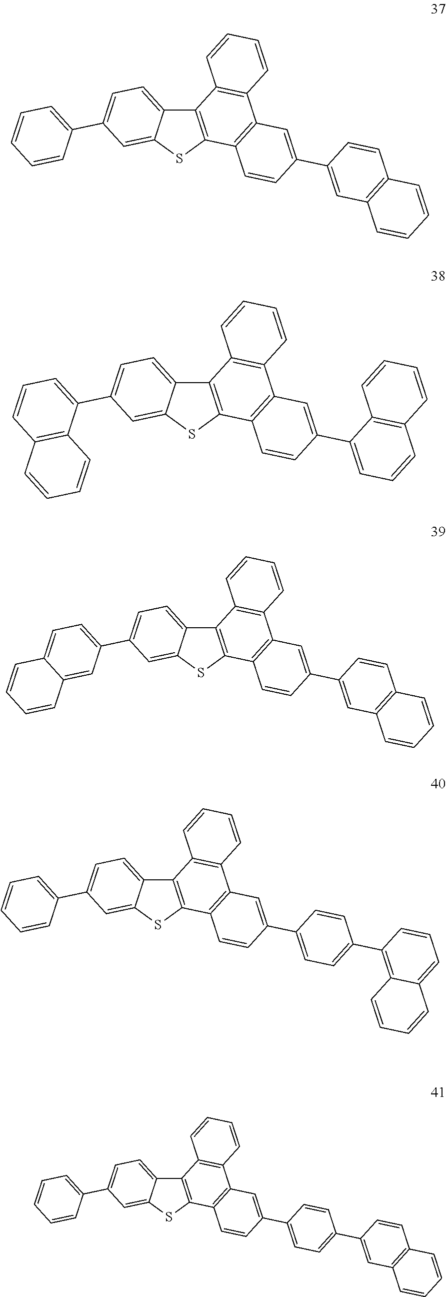 [SMILES: c1ccc(-c2ccc3c(c2)sc2c4ccc(-c5ccc6ccccc6c5)cc4c4ccccc4c32)cc1 ; c1ccc2c(-c3ccc4c(c3)sc3c5ccc(-c6cccc7ccccc67)cc5c5ccccc5c43)cccc2c1 ; c1ccc2cc(-c3ccc4c(c3)sc3c5ccc(-c6ccc7ccccc7c6)cc5c5ccccc5c43)ccc2c1 ; c1ccc(-c2ccc3c(c2)sc2c4ccc(-c5ccc(-c6cccc7ccccc67)cc5)cc4c4ccccc4c32)cc1 ; c1ccc(-c2ccc3c(c2)sc2c4ccc(-c5ccc(-c6ccc7ccccc7c6)cc5)cc4c4ccccc4c32)cc1]

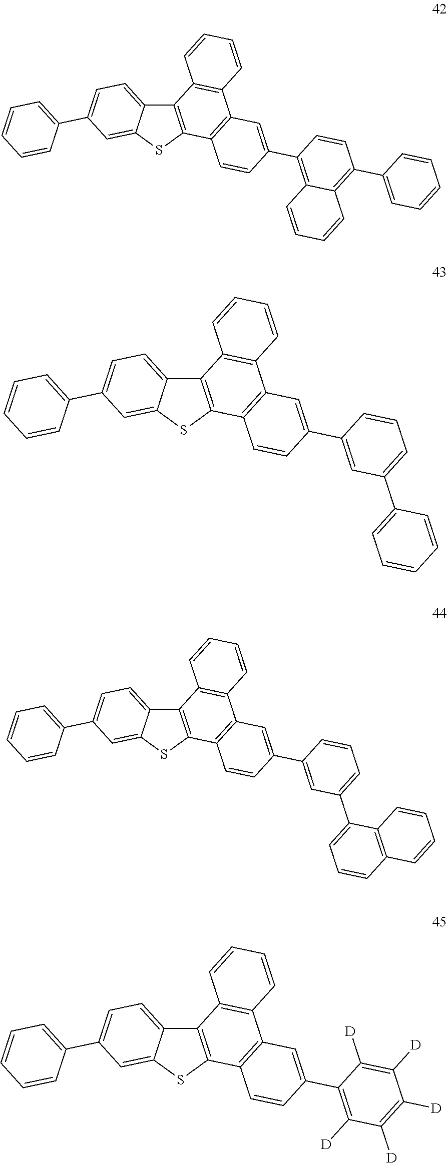 [SMILES: c1ccc(-c2ccc3c(c2)sc2c4ccc(-c5ccc(-c6ccccc6)c6ccccc56)cc4c4ccccc4c32)cc1 ; c1ccc(-c2cccc(-c3ccc4c(c3)c3ccccc3c3c5ccc(-c6ccccc6)cc5sc43)c2)cc1 ; c1ccc(-c2ccc3c(c2)sc2c4ccc(-c5cccc(-c6cccc7ccccc67)c5)cc4c4ccccc4c32)cc1 ; [2H]c1c([2H])c([2H])c(-c2ccc3c(c2)c2ccccc2c2c4ccc(-c5ccccc5)cc4sc32)c([2H])c1[2H]]

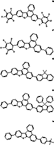 [SMILES: [2H]c1c([2H])c([2H])c(-c2ccc3c(c2)sc2c4ccc(-c5c([2H])c([2H])c([2H])c([2H])c5[2H])cc4c4ccccc4c32)c([2H])c1[2H] ; [2H]c1c([2H])c([2H])c(-c2ccc3c(c2)sc2c4ccc(-c5ccccc5)cc4c4ccccc4c32)c([2H])c1[2H] ; CC1(C)c2ccccc2-c2ccc(-c3ccc4c(c3)c3ccccc3c3c5ccc(-c6ccccc6)cc5sc43)cc21 ; c1ccc(-c2ccc3c(c2)sc2c4ccc(-c5ccc6c(c5)C(c5ccccc5)(c5ccccc5)c5ccccc5-6)cc4c4ccccc4c32)cc1 ; C[Si](C)(C)c1ccc(-c2ccc3c(c2)c2ccccc2c2c4ccc(-c5ccccc5)cc4sc32)cc1]

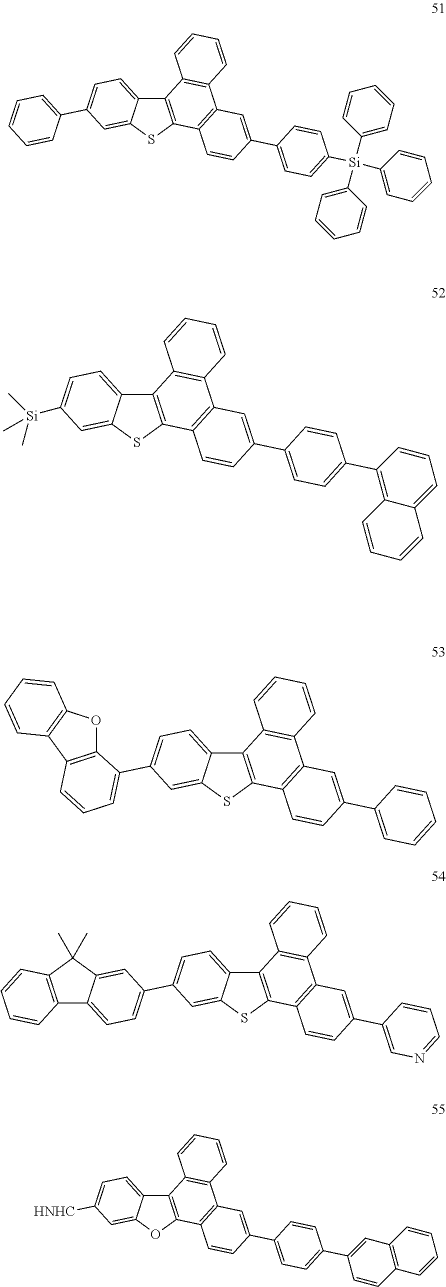

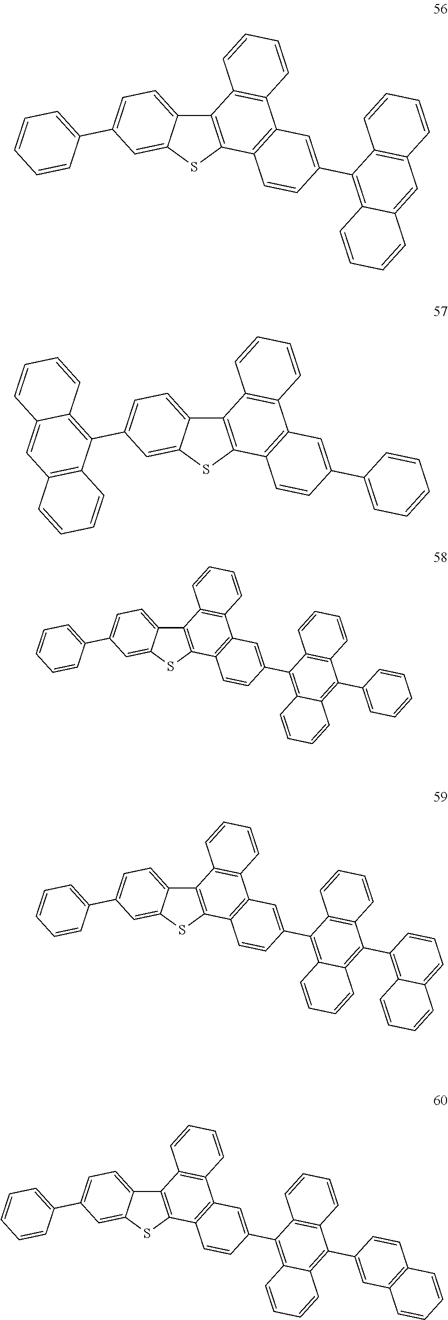 [SMILES: c1ccc(-c2ccc3c(c2)sc2c4ccc(-c5c6ccccc6cc6ccccc56)cc4c4ccccc4c32)cc1 ; c1ccc(-c2ccc3c(c2)c2ccccc2c2c4ccc(-c5c6ccccc6cc6ccccc56)cc4sc32)cc1 ; c1ccc(-c2ccc3c(c2)sc2c4ccc(-c5c6ccccc6c(-c6ccccc6)c6ccccc56)cc4c4ccccc4c32)cc1 ; c1ccc(-c2ccc3c(c2)sc2c4ccc(-c5c6ccccc6c(-c6cccc7ccccc67)c6ccccc56)cc4c4ccccc4c32)cc1 ; c1ccc(-c2ccc3c(c2)sc2c4ccc(-c5c6ccccc6c(-c6ccc7ccccc7c6)c6ccccc56)cc4c4ccccc4c32)cc1]

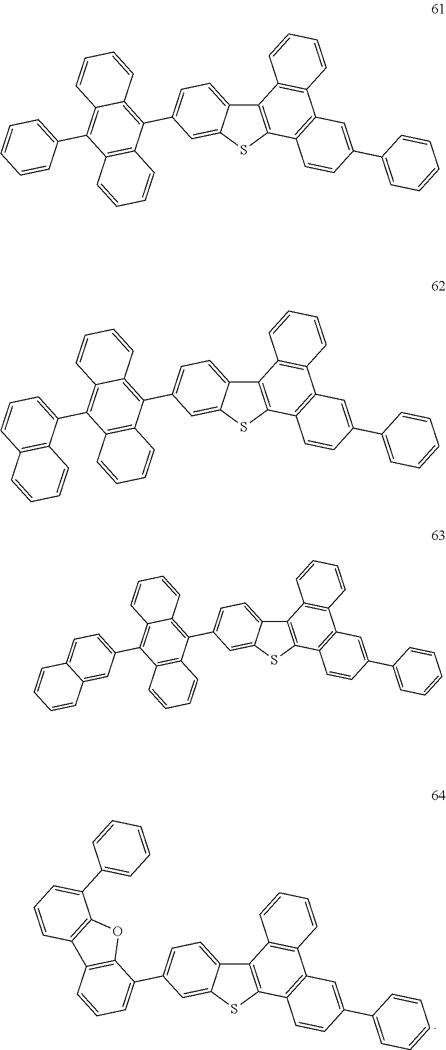 [SMILES: c1ccc(-c2ccc3c(c2)c2ccccc2c2c4ccc(-c5c6ccccc6c(-c6ccccc6)c6ccccc56)cc4sc32)cc1 ; c1ccc(-c2ccc3c(c2)c2ccccc2c2c4ccc(-c5c6ccccc6c(-c6cccc7ccccc67)c6ccccc56)cc4sc32)cc1 ; c1ccc(-c2ccc3c(c2)c2ccccc2c2c4ccc(-c5c6ccccc6c(-c6ccc7ccccc7c6)c6ccccc56)cc4sc32)cc1 ; c1ccc(-c2ccc3c(c2)c2ccccc2c2c4ccc(-c5cccc6c5oc5c(-c7ccccc7)cccc56)cc4sc32)cc1]

17. An organic light-emitting device, comprising
a first electrode;
a second electrode facing the first electrode; and
an organic layer between the first electrode and the second electrode, the organic layer including an emission layer,
wherein the organic layer includes the condensed cyclic compound of claim 1.

18. The organic light-emitting device as claimed in claim 17, wherein:
the first electrode is an anode,
the second electrode is a cathode, and
the organic layer includes:
a hole transport region between the first electrode and the emission layer, the hole transport region including at least one of a hole injection layer, a hole transport layer, a buffer layer, and an electron blocking layer, and
an electron transport region between the emission layer and the second electrode, the electron transport region including at least one of a hole blocking layer, an electron transport layer, and electron injection layer.

19. The organic light-emitting device as claimed in claim 17, wherein the emission layer includes the condensed cyclic compound represented by Formula 1.

20. The organic light-emitting device as claimed in claim 19, wherein the emission layer further includes a fluorescent dopant.
